(19) United States
(12) Patent Application Publication
(10) Pub. No.: US 2024/0122981 A1
(43) Pub. Date: Apr. 18, 2024

(54) CCR4-Targeting Chimeric Antigen Receptor Cell Therapy

(71) Applicants: THE TRUSTEES OF THE UNIVERSITY OF PENNSYLVANIA, PHILADELPHIA, PA (US); NATIONIAL CANCER CENTER, Tokyo (JP)

(72) Inventors: Carl H. June, Merion Station, PA (US); Keisuke Watanabe, Nagoya (JP); Regina M. Young, Bryn Mawr, PA (US); John Scholler, Ardmore, PA (US); Hiroyoshi Nishikawa, Ardmore, PA (US)

(21) Appl. No.: 18/276,582

(22) PCT Filed: Feb. 10, 2022
(86) PCT No.: PCT/US2022/015981

§ 371 Date: Aug. 9, 2023

Related U.S. Application Data
(60) Provisional application No. 63/148,489, filed on Feb. 11, 2021.

(51) Int. Cl. A61K 35/17; A61K 39/00; A61P 35/00; C07K 16/28

(52) U.S. Cl. CPC A61K 35/17; A61K 39/4611; A61P 35/00; C07K 16/2866; A61K 2039/5156; A61K 2239/13; A61K 2239/21; C07K 2317/565; C07K 2317/622

(57) ABSTRACT

The present disclosure provides anti-CCR4 chimeric antigen receptors (CARs) and compositions and methods for modified immune cells or precursors thereof (e.g., modified T cells) comprising anti-CCR4 CARs. Also provided are methods of using the anti-CCR4 CAR-expressing cells to treat cancer and T cell-depleting systems for use in combination with anti-CCR4 CAR T cell therapy.

### CROSS-REFERENCE TO RELATED APPLICATIONS

This application claims priority under 35 U.S.C. § 119(e) to U.S. Provisional Patent Application No. 63/148,489, filed Feb. 11, 2021, which is incorporated herein by reference in its entirety.

### BACKGROUND OF THE INVENTION

A challenge when targeting T cell lymphoma by chimeric antigen receptor (CAR) T cell therapy is that target antigens are often shared between T cells and tumor cells, resulting in fratricidal loss of normal T cells and CAR T cell product. CC chemokine receptor 4 (CCR4) is a G protein-coupled seven transmembrane domain chemokine receptor that binds the chemokines CCL17/TARC and CCL22/MDC1. CCR4 is highly expressed by many mature T-cell malignancies such as a majority of adult T-cell leukemia/lymphoma (ATLL), cutaneous T-cell lymphomas (CTCLs) and 30-40% of peripheral T cell lymphoma (PTCL) cases, and has a unique expression profile on normal T cells. Specifically, CCR4 is dominantly expressed by type-2 and type-17 helper T cell (Th2 and Th17), effector-type regulatory-T cell (Treg) subsets and cutaneous lymphocyte antigen (CLA)+ skin-homing T cells, but rarely expressed by the other helper T cell subsets and CD8+ T cells. All T cell malignancies which express CCR4 show poor prognosis and curative treatment is rare. In addition, CCR4 on tumors is functional and its expression is associated with a poorer disease outcome. As such, CCR4 is a potential target for the treatment of mature T cell malignancies.

Feasibility of CCR4 targeting therapy has been suggested in the context of monoclonal antibody (mAb) treatment. Mogamulizumab (KW-0761) is a defucosylated humanized anti-CCR4 mAb designed to induce enhanced antibody-dependent-cellular-cytotoxicity (ADCC). Phase I/II trial of mogamulizumab for CTCL or ATLL showed acceptable safety profile and some efficacy, and a phase III trial demonstrated that mogamulizumab significantly prolonged progression-free survival of patients with CTCL compared with vorinostat, one of the standard therapies for CTCL. Adverse events were comparable to vorinostat and most of them were manageable although a case of severe cutaneous hypersensitivity reaction (Stevens-Johnson syndrome) has been reported. Based on these trials, mogamulizumab has been approved by Pharmaceuticals and Medical Devices Agency (PMDA) for the treatment of refractory ATLL, CTCL and PTCL in Japan and recently approved by FDA for CTCL in the US. However, the majority of patients treated with mogamulizumab experience recurrence, thus cure is rarely achieved. Additionally, a previous report suggested that functional CCR4-redirected chimeric antigen receptor (CCR4-CAR) T cells can be established (Perera L P, et al. Am J Hematol. 2017; 92(9):892-901). However precise mechanisms of the fratricidal event and its relevance to T cell expansion, transduction, functions, target sensing, phenotype and anti-tumor efficacy remain unclear. Thus, there is a need in the art for novel anti-CCR4 CAR-based therapies for T cell malignancies that overcome these obstacles and challenges. The present invention addresses this need.

### SUMMARY OF THE INVENTION

In some aspects, the invention provides an anti-CC chemokine receptor 4 (CCR4) chimeric antigen receptor (CAR) comprising an anti-CCR4 antigen binding domain, a transmembrane domain, and an intracellular domain.

In some embodiments, the anti-CCR4 antigen binding domain comprises at least one heavy chain variable region (HCDR) comprising the amino acid sequence selected from the group consisting of SEQ ID NO: 2, SEQ ID NO: 4, and SEQ ID NO: 6.

In some embodiments, the anti-CCR4 antigen binding domain comprises at least one light chain variable region (LCDR) comprising the amino acid sequence selected from the group consisting of SEQ ID NO: 8, SEQ ID NO: 10, and SEQ ID NO: 12.

In some embodiments, the anti-CCR4 antigen binding domain is selected from the group consisting of a full length antibody or antigen-binding fragment thereof, a Fab, a single-chain variable fragment (scFv), or a single-domain antibody.

In some embodiments, the anti-CCR4 antigen binding domain is an anti-CCR4 single-chain variable fragment (scFv).

In some embodiments, the anti-CCR4 antigen binding domain is an anti-CCR4 single-chain variable fragment (scFv) having a heavy chain and a light chain derived from mogamulizumab.

In some embodiments, the anti-CCR4 antigen binding domain comprises a heavy chain variable region comprising an amino acid sequence at least 80%, 85%, 90%, 95%, 96%, 97%, 98%, 99%, or 100% identical to SEQ ID NO: 112.

In some embodiments, the anti-CCR4 antigen binding domain comprises a light chain variable region comprising an amino acid sequence at least 80%, 85%, 90%, 95%, 96%, 97%, 98%, 99%, or 100% identical to SEQ ID NO: 114.

In some embodiments, the anti-CCR4 antigen binding domain comprises an amino acid sequence at least 80%, 85%, 90%, 95%, 96%, 97%, 98%, 99%, or 100% identical to SEQ ID NO: 16.

In some embodiments, the CAR further comprises a CD8 alpha hinge, optionally wherein the CD8 alpha hinge comprises the amino acid sequence set forth in SEQ ID NO: 34.

In some embodiments, the transmembrane domain comprises a transmembrane domain selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS, and CD154, or a transmembrane domain derived from a killer immunoglobulin-like receptor (KIR).

In some embodiments, the transmembrane domain comprises a CD8 alpha transmembrane domain, optionally wherein the CD8 alpha transmembrane domain comprises the amino acid sequence set forth in SEQ ID NO: 36.

In some embodiments, the intracellular domain comprises a costimulatory domain and an intracellular signaling domain.

In some embodiments, the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain comprises a costimulatory domain of 4-1BB optionally comprising the amino acid sequence set forth in SEQ ID NO: 38.

In some embodiments, the intracellular domain comprises an intracellular signaling domain of a protein selected from the group consisting of a human CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular signaling domain comprises an intracellular signaling domain of CD3ζ or a variant thereof, optionally comprising the amino acid sequence set forth in SEQ ID NO: 40.

In some embodiments, the CAR comprises an anti-CCR4 antigen binding domain comprising an anti-CCR4 scFv, a CD8 alpha hinge, a CD8 alpha transmembrane domain, an intracellular domain comprising a 4-1BB costimulatory domain and a CD3 intracellular signaling domain.

In some embodiments, the CAR further comprises a CD8 leader sequence, optionally comprising the amino acid sequence set forth in SEQ ID NO: 32.

In some embodiments, the CAR comprises an amino acid sequence at least 80%, 85%, 90%, 95%, 96%, 97%, 98%, 99%, or 100% identical to any one of SEQ ID NOs: 30, 54, 62, 70, 82, 90, and 102.

In some embodiments, the CAR is encoded by a nucleotide sequence at least 80%, 85%, 90%, 95%, 96%, 97%, 98%, 99%, or 100% identical to any one of SEQ ID NOs: 29, 53, 61, 69, 81, 89, and 101.

In some embodiments, the CAR is linked to a safety switch via a self-cleavable linker, and wherein the safety switch binds a safety switch agent.

In some embodiments, the safety switch is selected from: (a) truncated EGFR (EGFRt), wherein the safety switch agent is an anti-EFGR antibody, optionally cetuximab; (b) CD20, wherein the safety switch agent is an anti-CD20 antibody, optionally rituximab; (c) inducible caspase 9 (iCasp9), wherein the safety switch agent is AP1903; and (d) Herpes simplex virus-1 thymidine kinase (HSVTK), wherein the safety switch agent is ganciclovir (GCV).

In some embodiments, the CAR is linked to a dominant negative TGFb receptor type II (dnTGFbRII) via a self-cleavable linker.

In some embodiments, the safety switch is linked to a dominant negative TGFb receptor type II (dnTGFbRII) via a self-cleavable linker.

In some aspects, the invention provides a nucleic acid comprising a polynucleotide sequence encoding an anti-CCR4 CAR, wherein the CAR comprises an anti-CCR4 antigen binding domain, a transmembrane domain, and an intracellular domain.

In some embodiments, the anti-CCR4 antigen binding domain comprises at least one heavy chain variable region (HCDR) comprising the amino acid sequence selected from the group consisting of SEQ ID NO: 2, SEQ ID NO: 4, and SEQ ID NO: 6.

In some embodiments, the anti-CCR4 antigen binding domain comprises at least one light chain variable region (LCDR) comprising the amino acid sequence selected from the group consisting of SEQ ID NO: 8, SEQ ID NO: 10, and SEQ ID NO: 12.

In some embodiments, the anti-CCR4 antigen binding domain is selected from the group consisting of a full length antibody or antigen-binding fragment thereof, a Fab, a single-chain variable fragment (scFv), or a single-domain antibody.

In some embodiments, the anti-CCR4 antigen binding domain is an anti-CCR4 single-chain variable fragment (scFv).

In some embodiments, the anti-CCR4 antigen binding domain is an anti-CCR4 single-chain variable fragment (scFv) having a heavy chain and a light chain derived from mogamulizumab.

In some embodiments, the anti-CCR4 antigen binding domain comprises a heavy chain variable region comprising an amino acid sequence at least 80%, 85%, 90%, 95%, 96%, 97%, 98%, 99%, or 100% identical to SEQ ID NO: 112.

In some embodiments, the anti-CCR4 antigen binding domain comprises a light chain variable region comprising an amino acid sequence at least 80%, 85%, 90%, 95%, 96%, 97%, 98%, 99%, or 100% identical to SEQ ID NO: 114.

In some embodiments, the anti-CCR4 antigen binding domain comprises an amino acid sequence at least 80%, 85%, 90%, 95%, 96%, 97%, 98%, 99%, or 100% identical to SEQ ID NO: 16.

In some embodiments, the CAR further comprises a CD8 alpha hinge, optionally wherein the CD8 alpha hinge comprises the amino acid sequence set forth in SEQ ID NO: 34.

In some embodiments, the transmembrane domain comprises a transmembrane domain selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS, and CD154, or a transmembrane domain derived from a killer immunoglobulin-like receptor (KIR).

In some embodiments, the transmembrane domain comprises a CD8 alpha transmembrane domain, optionally wherein the CD8 alpha transmembrane domain comprises the amino acid sequence set forth in SEQ ID NO: 36.

In some embodiments, the intracellular domain comprises a costimulatory domain and an intracellular signaling domain.

In some embodiments, the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain comprises a costimulatory domain of 4-1BB optionally comprising the amino acid sequence set forth in SEQ ID NO: 38.

In some embodiments, the intracellular domain comprises an intracellular signaling domain of a protein selected from the group consisting of a human CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular signaling domain comprises an intracellular signaling domain of CD3ζ or a variant thereof, optionally comprising the amino acid sequence set forth in SEQ ID NO: 40.

In some embodiments, the CAR comprises an anti-CCR4 antigen binding domain comprising an anti-CCR4 scFv, a CD8 alpha hinge, a CD8 alpha transmembrane domain, an intracellular domain comprising a 4-1BB costimulatory domain and a CD3 intracellular signaling domain.

In some embodiments, the CAR further comprises a CD8 leader sequence, optionally comprising the amino acid sequence set forth in SEQ ID NO: 32.

In some embodiments, the CAR comprises an amino acid sequence at least 80%, 85%, 90%, 95%, 96%, 97%, 98%, 99%, or 100% identical to any one of SEQ ID NOs: 30, 54, 62, 70, 82, 90, and 102.

In some embodiments, the CAR is encoded by a nucleotide sequence at least 80%, 85%, 90%, 95%, 96%, 97%, 98%, 99%, or 100% identical to any one of SEQ ID NOs: 29, 53, 61, 69, 81, 89, and 101.

In some embodiments, the CAR is linked to a safety switch via a self-cleavable linker, and wherein the safety switch binds a safety switch agent.

In some embodiments, the safety switch is selected from: (a) truncated EGFR (EGFRt), wherein the safety switch agent is an anti-EFGR antibody, optionally cetuximab; (b) CD20, wherein the safety switch agent is an anti-CD20 antibody, optionally rituximab; (c) inducible caspase 9 (iCasp9), wherein the safety switch agent is AP1903; and (d) Herpes simplex virus-1 thymidine kinase (HSVTK), wherein the safety switch agent is ganciclovir (GCV).

In some embodiments, the CAR is linked to a dominant negative TGFb receptor type II (dnTGFbRII) via a self-cleavable linker.

In some embodiments, the safety switch is linked to a dominant negative TGFb receptor type II (dnTGFbRII) via a self-cleavable linker.

In some aspects, the invention provides a vector comprising the nucleic acid described herein.

In some embodiments, the vector is a viral vector selected from the group consisting of a retroviral vector, a lentiviral vector, an adenoviral vector, and an adeno-associated viral vector.

In some embodiments, the viral vector is a lentiviral vector.

In some aspects, the invention provides a modified immune cell or precursor cell thereof, comprising the CAR described herein, the nucleic acid described herein, and/or the vector described herein.

In some embodiments, the modified immune cell or precursor cell thereof further comprises one or more of the following;

(a) a CCR4 null knockout allele;

(b) suppressed CCR4 gene expression; and

(c) a fusion protein comprising an anti-CCR4 scFv and a KDEL motif and/or a nucleic acid encoding the fusion protein.

In some embodiments, the CCR4 null knockout allele or suppressed CCR4 gene expression is obtained via a genetic engineering technique comprising a nuclease selected from the group consisting of a clustered regularly interspaced short palindromic repeats (CRISPR) associated nuclease, a transcription activator-like effector nuclease (TALEN), and a zinc-finger nuclease.

In some embodiments, the modified immune cell or precursor cell thereof further comprises an inhibitory RNA molecule which suppresses CCR4 gene expression.

In some embodiments, the inhibitory RNA molecule is selected from the group consisting of: an RNA interference (RNAi) RNA, a short hairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.

In some embodiments, the modified cell is an autologous cell.

In some embodiments, the modified immune cell is derived from peripheral blood mononuclear cells (PBMCs).

In some embodiments, the PBMCs comprise tumor cells.

In some embodiments, the modified immune cell or precursor cell thereof is a cell isolated from a human subject.

In some embodiments, the modified immune cell is a modified T cell.

In some aspects, the invention provides a method for generating a modified immune cell or precursor cell thereof, comprising introducing into the immune or precursor cell thereof the nucleic acid described herein or the vector described herein.

In some embodiments, the vector is introduced via viral transduction.

In some aspects, the invention provides a method of treating cancer in a subject in need thereof, comprising administering to the subject the modified immune or precursor cell of any one of embodiments 54-63, or a modified immune or precursor cell thereof generated by the method of embodiment 64 or embodiment 65.

In some embodiments, the modified cell depletes CCR4-positive T cells and not CCR4-negative T cells in the subject, thereby treating the cancer.

In some embodiments, the subject has previously been administered mogamulizumab.

In some embodiments, the cancer is refractory to mogamulizumab.

In some aspects, the invention provides a method of treating cancer in a subject in need thereof, comprising administering to the subject a modified T cell comprising an anti-CCR4 CAR, wherein the CAR comprises an anti-CCR4 antigen binding domain, a transmembrane domain, and an intracellular domain.

In some embodiments, the anti-CCR4 antigen binding domain comprises at least one heavy chain variable region (HCDR) comprising the amino acid sequence selected from the group consisting of SEQ ID NO: 2, SEQ ID NO: 4, and SEQ ID NO: 6.

In some embodiments, the anti-CCR4 antigen binding domain comprises at least one light chain variable region (LCDR) comprising the amino acid sequence selected from the group consisting of SEQ ID NO: 8, SEQ ID NO: 10, and SEQ ID NO: 12.

In some embodiments, the anti-CCR4 antigen binding domain is selected from the group consisting of a full length antibody or antigen-binding fragment thereof, a Fab, a single-chain variable fragment (scFv), or a single-domain antibody.

In some embodiments, the anti-CCR4 antigen binding domain is an anti-CCR4 single-chain variable fragment (scFv).

In some embodiments, the anti-CCR4 antigen binding domain is an anti-CCR4 single-chain variable fragment (scFv) having a heavy chain and a light chain derived from mogamulizumab.

In some embodiments, the anti-CCR4 antigen binding domain comprises a heavy chain variable region comprising an amino acid sequence at least 80%, 85%, 90%, 95%, 96%, 97%, 98%, 99%, or 100% identical to SEQ ID NO: 112.

In some embodiments, the anti-CCR4 antigen binding domain comprises a light chain variable region comprising an amino acid sequence at least 80%, 85%, 90%, 95%, 96%, 97%, 98%, 99%, or 100% identical to SEQ ID NO: 114.

In some embodiments, the anti-CCR4 antigen binding domain comprises an amino acid sequence at least 80%, 85%, 90%, 95%, 96%, 97%, 98%, 99%, or 100% identical to SEQ ID NO: 16.

In some embodiments, the CAR further comprises a CD8 alpha hinge, optionally wherein the CD8 alpha hinge comprises the amino acid sequence set forth in SEQ ID NO: 34.

In some embodiments, the transmembrane domain comprises a transmembrane domain selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS, and CD154, or a transmembrane domain derived from a killer immunoglobulin-like receptor (KIR).

In some embodiments, the transmembrane domain comprises a CD8 alpha transmembrane domain, optionally wherein the CD8 alpha transmembrane domain comprises the amino acid sequence set forth in SEQ ID NO: 36.

In some embodiments, the intracellular domain comprises a costimulatory domain and an intracellular signaling domain.

In some embodiments, the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain comprises a costimulatory domain of 4-1BB optionally comprising the amino acid sequence set forth in SEQ ID NO: 38.

In some embodiments, the intracellular domain comprises an intracellular signaling domain of a protein selected from the group consisting of a human CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular signaling domain comprises an intracellular signaling domain of CD3ζ or a variant thereof, optionally comprising the amino acid sequence set forth in SEQ ID NO: 40.

In some embodiments, the CAR comprises an anti-CCR4 antigen binding domain comprising an anti-CCR4 scFv, a CD8 alpha hinge, a CD8 alpha transmembrane domain, an intracellular domain comprising a 4-1BB costimulatory domain and a CD3 intracellular signaling domain.

In some embodiments, the CAR further comprises a CD8 leader sequence, optionally comprising the amino acid sequence set forth in SEQ ID NO: 32.

In some embodiments, the CAR comprises an amino acid sequence at least 80%, 85%, 90%, 95%, 96%, 97%, 98%, 99%, or 100% identical to any one of SEQ ID NOs: 30, 54, 62, 70, 82, 90, and 102.

In some embodiments, the CAR is encoded by a nucleotide sequence at least 80%, 85%, 90%, 95%, 96%, 97%, 98%, 99%, or 100% identical to any one of SEQ ID NOs: 29, 53, 61, 69, 81, 89, and 101.

In some embodiments, the CAR is linked to a safety switch via a self-cleavable linker, and wherein the safety switch binds a safety switch agent.

In some embodiments, the safety switch is selected from: (a) truncated EGFR (EGFRt), wherein the safety switch agent is an anti-EFGR antibody, optionally cetuximab; (b) CD20, wherein the safety switch agent is an anti-CD20 antibody, optionally rituximab; (c) inducible caspase 9 (iCasp9), wherein the safety switch agent is AP1903; and (d) Herpes simplex virus-1 thymidine kinase (HSVTK), wherein the safety switch agent is ganciclovir (GCV).

In some embodiments, the CAR is linked to a dominant negative TGFb receptor type II (dnTGFbRII) via a self-cleavable linker.

In some embodiments, the safety switch is linked to a dominant negative TGFb receptor type II (dnTGFbRII) via a self-cleavable linker.

In some embodiments, the method further comprises administering the safety switch agent to the subject.

In some embodiments, the modified T cell depletes CCR4-positive T cells and not CCR4-negative T cells in the subject, thereby treating the cancer.

In some embodiments, the modified T cell is derived from PBMCs.

In some embodiments, the PBMCs comprise tumor cells.

In some embodiments, the modified T cell is a human T cell.

In some embodiments, the modified T cell is autologous.

In some embodiments, the subject is human.

In some embodiments, the subject has previously been administered mogamulizumab.

In some embodiments, the cancer is refractory to mogamulizumab.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 illustrates the finding that subcutaneous T cell lymphoma cells express high levels of CCR4. CCR4 expression on skin biopsy samples from four patients with CTCL were evaluated with immunohistochemistry (IHC). Each scale bar represents 100 um.

FIG. 2 illustrates the finding that normal tissues rarely express CCR4. CCR4 expression on normal tissue microarray (TMA) for 27 organs (triplicate) were analyzed. Lower table represents the map of TMA.

FIG. 3 depicts a schematic representation of CCR4-CAR construct. CD8 leader, CD8 leader sequence; scFv, single chain variable fragment; TM, transmembrane domain.

FIG. 4 illustrates the finding that CCR4-CAR T cells can expand while depleting CCR4 expressing T cells. Expansion of T cells transduced with CCR4-CAR for 21 days (left panel) and total doublings at day 10 (right panel). Left panel shows the representative T cell expansion curves. T cells were counted and diluted to 0.7×10⁶ cells/ml by adding fresh media every to every other day. T cells were harvested when the T cell sizes went down to 350 um³ for the subsequent experiments, when a part of cells were spared to continue counting. Right panel shows pooled data of total T cell expansion at day 10 post anti-CD3/28 stimulation. Dots and bars represent means and standard error of means (SEM) (n=5). Data are representative (left panel) or pooled (right panel) from 5 experiments from 5 donors. ns, not significant; *, p<0.05; **, p<0.01; ***, p<0.001; ****, p<0.0001 (v.s. CD19CART group) by one-way ANOVA with Tukey's post-hoc test.

FIG. 5 depicts final yield of CAR T cell product. T cells were stimulated with anti-CD3/CD28 beads then transduced either with CCR4-CAR, control CD19-CAR or untraduced. T cells were harvested and frozen when the cell sizes went down to 350 um³ for subsequent experiments (typically between day 10 and 14 post beads stimulation). Overall doublings of T cells at the day of harvesting are shown. Bars represent mean and SEM. Data are pooled of 5 experiments from 5 donors. ns, not significant; *, p<0.05; ***, p<0.001 (v.s. 19CART group) by one-way ANOVA with Tukey's post-hoc test.

FIG. 6 depicts T cell size during CAR T cell expansion. T cell size were analyzed every to every other day by a cell counter (Coulter counter) (right panel). Size at day 10 from pooled data are shown in right panel. Data are representative or pooled of 5 experiments from 5 donors.

FIG. 7 illustrates the finding that CCR4-CAR T cells can expand while depleting CCR4 expressing T cells. Time course analysis of T cell viability. Cell viability was analyzed by amine reactive dye staining (LIVE-DEAD staining) and FCM. Dots and bars represent means and SEM (n=5). Statistical analysis was applied to data at day 10. Data are pooled from 5 experiments from 5 donors. ns, not significant; *, p<0.05; **, p<0.01; ***, p<0.001; ****, p<0.0001 (v.s. CD19CART group) by one-way ANOVA with Tukey's post-hoc test.

FIG. 8 depicts T cell memory phenotype by CD45RO and CCR7 expression at day 10. Numbers denote percentage of cells in each quadrant.

FIG. 9 illustrates the finding that CCR4-CAR T cells can expand while depleting CCR4 expressing T cells. Time course analysis of CAR expression of T cells showing CAR positive cell selection in some types of CAR T cells. Dots and bars represent means and SEM (n=5). Statistical analysis was applied to data at day 10. Data are pooled from 5 experiments from 5 donors. ns, not significant; *, p<0.05; **, p<0.01; ***, p<0.001; ****, p<0.0001 (v.s. CD19CART group) by one-way ANOVA with Tukey's post-hoc test.

FIG. 10 depicts expression of CARs on the surface of T cells. CAR expression was analyzed at day 10 post stimulation by FCM. Gates represents CAR positive population and numbers denote CAR positive percentages.

FIG. 11 depicts anti-CCR4 mAbs used for CCR4-CAR construction do not share the epitope of CCR4 with the detection antibody. CCR4 on HH cells was blocked either with purified CCR4 mAb clones KW-0761 or h1567, and then stained either with clones KW-0761 (Ax647 conjugated), h1567 (PE conjugated) or D8SEE (PE conjugated). CCR4 was analyzed by FCM. CCR4 was still detectable even after CCR4 blocking with clones KW-0761 and h1567 although KW-0761 slightly decreased detection efficiency by the detection mAb (D8SEE).

FIG. 12 illustrates the finding that CCR4-CAR T cells can expand while depleting CCR4 expressing T cells. Representative flow plots showing expression of CAR and CCR4 on T cells at day 6 post stimulation. Numbers denote percentage of cells in each quadrant. Data are representative from 5 experiments from 5 donors. ns, not significant; *, p<0.05; **, p<0.01; ***, p<0.001; ****, p<0.0001 (v.s. CD19CART group) by one-way ANOVA with Tukey's post-hoc test.

FIG. 13 illustrates the finding that CCR4-CAR T cells can expand while depleting CCR4 expressing T cells. Representative kinetics of CCR4 expression (left panels) and pooled data of CCR4 expression at day 10 (right panel). Bars represent mean and SEM (n=5). Data are representative (left panel) or pooled (right panel) from 5 experiments from 5 donors. ns, not significant; *, p<0.05; **, p<0.01; ***, p<0.001; ****, p<0.0001 (v.s. CD19CART group) by one-way ANOVA with Tukey's post-hoc test.

FIG. 14 depicts change of CD4/8 ratio during CAR T cell expansion. CD4/8 ratio in CAR positive population (upper panel) and CAR negative population (lower panel) at each time point was analyzed by FCM. Data are pooled of 5 experiments from 5 donors. ns, not significant; *, p<0.05; **, p<0.01 (v.s. 19CART group at day 10) by one-way ANOVA with Tukey's post-hoc test.

FIG. 15 depicts HH cells transduced with two different CCR4-CARs; CCR4-CAR(KW_L2H) and CCR4-CAR(h1567_L2H) (related to FIG. 16). CCR4-CAR expression on HH cells were analyzed by FCM.

FIG. 16 illustrates the finding that CCR4-CAR T cells can expand while depleting CCR4 expressing T cells. Epitope specific blocking of CCR4 by CCR4-CAR on HH cells. CCR4 expression detected either with anti-CCR4mAb clone h1567 (left panel) or anti-CCR4 mAb clone KW-0761 (right panel) on HH cells transduced with indicated CCR4 CAR is shown. Data are representative of 2 experiments. ns, not significant; *, p<0.05; **, p<0.01; ***, p<0.001; ****, p<0.0001 (v.s. CD19CART group) by one-way ANOVA with Tukey's post-hoc test.

FIG. 17 illustrates the finding that fratricide changes Th subset proportion with decreased Th2, Th17 and Treg cytokine production while spared Th1 cytokine production. Ratio of Th1 subset and Th2 subset of CAR T cell products. Th1 subset was defined as CD4(+), CXCR3(+), CCR4(−), CCR6(−) and CCR10(−). Th2 subset was defined as CD4(+), CXCR3(−), CCR4(+), CCR6(−) and CCR10(−) by FCM. Phenotypes were analyzed by gating CAR positive population with exception of UTD group. Data are pooled from 3 experiments from 3 donors. Bars represent means and SEM (n=3). ns, not significant; ***, p<0.001; ****, p<0.0001 (v.s. CD19-CAR T group) by one-way ANOVA with Tukey's post-hoc test.

FIG. 18 illustrates the finding that fratricide changes Th subset proportion with decreased Th2, Th17 and Treg cytokine production while spared Th1 cytokine production. Heat map of Th-related cytokine production by CCR4-CAR T cells relative to CD19-CAR T cells. CAR T cells were stimulated with PMA/Iono. Cytokine levels of supernatant at 24 hours post stimulation were analyzed by LUMINEX assay. Cytokine levels from CD19-CAR T cells were set to one. Data are representative of 2 experiments from 2 donors.

FIG. 19 illustrates the finding that fratricide changes Th subset proportion with decreased Th2, Th17 and Treg cytokine production while spared Th1 cytokine production. Depletion of CCR4 positive subsets by adding CCR4-CAR T cells to CD19-CAR T cells. T cells were stimulated and transduced with either CCR4 CAR or CD19 CAR separately. 10% number of CCR4-CAR T cells were added to CD19-CAR T cells at day5 after stimulation. CCR4 expression in CD19-CAR positive cells were analyzed at day10. Numbers denote percentage of cells in each quadrant. Data are representative of 2 experiments from 2 donors.

FIG. 20 illustrates the finding that fratricide changes Th subset proportion with decreased Th2, Th17 and Treg cytokine production while spared Th1 cytokine production. Heat map of Th-related cytokine production by CD19-CAR T cells pre-treated with CCR4-CAR T cells relative to untreated CD19-CAR T cells. CAR T cells were stimulated with the indicated stimulator. Cytokine levels of supernatant at 24 hours post stimulation were analyzed by LUMINEX assay. Cytokine levels from untreated CD19-CAR T cells were set to one.

FIG. 21 depicts expression of CCR4 on tumor cell lines by FCM. Numbers in each column denote MFI of CCR4-PE. Nalm6, HH, MJ, and HuT78 are cell lines derived from patients with B-ALL, leukemic CTCL, Sezary syndrome, and HTLV-1 positive MF respectively.

FIG. 22 depicts lytic activity of CAR T cells against tumor cell lines. CAR T cells and tumor cells expressing luciferase were incubated at the indicated E:T ratio. Specific lysis at 16 hours of co-culture was determined based on luminescence. Bars represent means and SD (triplicate). Data are representative of 3 experiments from 3 donors.

FIG. 23 depicts degranulation and cytokine production by CCR4-CAR T cells. CAR T cells were stimulated either with media, PMA-Iono or the indicated cells at 1:4 R:S ratio in the existence of monensin and CD107a detecting antibody. Cells were stained for intracellular cytokines after 6 hours co-culture using FoxP3 staining buffer set and analyzed by FCM. Bars represent means and SD (duplicate). Data are representative of 3 experiments from 3 donors.

FIG. 24 illustrates the finding that CCR4-CAR(KW_L2H) cures HH cell xenograft mice and induce superior T cell engraftment. Experiment schematic. NSG mice were intravenously inoculated with 1×10⁶ HH cells expressing luciferase (HH-CGB-GFP). Engrafted tumors were intravenously treated with either 0.5×10⁶ CCR4-CAR positive T cells or un-transduced T cells 7 days post tumor cell inoculation. Data are representative of 2 experiments from 2 donors.

FIG. 25 illustrates the finding that CCR4-CAR(KW_L2H) cures HH cell xenograft mice and induce superior T cell engraftment. Overall kinetics of tumor burden by BLI. The dash line represents the background level of photons determined by imaging no tumor mice. Dots and bars represent means and SEM (n=4 for UTD group and n=5 for the other groups). Data are representative of 2 experiments from 2 donors.

FIG. 26 illustrates the finding that CCR4-CAR(KW_L2H) cures HH cell xenograft mice and induce superior T cell engraftment. Survival curve by Kaplan-Meier method. *, p<0.05; **, p<0.01 (v.s. KW_L2H group) by Log-rank test. Data are representative of 2 experiments from 2 donors.

FIG. 27 illustrates the finding that CCR4-CAR(KW_L2H) cures HH cell xenograft mice and induce superior T cell engraftment. T cell count of peripheral blood at day 12 and 19 by FCM. *, p<0.05; **, p<0.01; ***, p<0.001; ****, p<0.0001 (v.s. KW_L2H group) by one-way ANOVA with Tukey's post-hoc test. Data are representative of 2 experiments from 2 donors.

FIG. 28 depicts BW change during CCR4-CAR T cell treatment. Dots and bars represent means and SEM (n=4 for UTD group and n=5 for the other groups). Some mice showed rapid BW gain within 30 days after treatment, which reflected hepatosplenomegaly caused by tumor invasion. Slow BW gain of the KW_L2H group was related with normal growth of mice as all the mice from the KW_L2H group kept CR during treatment.

FIG. 29 depicts CD19 expression on HH cells transduced with truncated CD19. HH cells were transduced with truncated CD19 and sorted repeatedly to purify CD19 positive population. Dot plots are showing 100% purity of CD19 expression in FCM. Gates represent CD19 positive population and numbers denote percentage of positive cells (left panel) or MFI of PE (right panel).

FIG. 30 illustrates the finding that CCR4-CAR(KW_L2H) induces antitumor efficacy comparable to that of CD19-CAR in NSG mice engrafted with HH expressing CD19 (HH-CBG-GFP-t19). Experiment schematic. NSG mice were intravenously inoculated with 1×10⁶ HH cells expressing luciferase and truncated CD19 (HH-CGB-GFP-t19). Engrafted tumors were intravenously treated with either 2×10⁶ CCR4-CAR positive T cells or UTD T cells 7 days post tumor cell inoculation.

FIG. 31 illustrates the finding that CCR4-CAR(KW_L2H) induces antitumor efficacy comparable to that of CD19-CAR in NSG mice engrafted with HH expressing CD19 (HH-CBG-GFP-t19). Overall kinetics of tumor burden by BLI. Dash line represents the background level of photons determined by imaging no tumor mice. Dots and bars represent means and SEM (n=5 for UTD group and n=7 for the other groups). Data are representative of 2 experiments from 2 donors.

FIG. 32 illustrates the finding that CCR4-CAR(KW_L2H) induces antitumor efficacy comparable to that of CD19-CAR in NSG mice engrafted with HH expressing CD19 (HH-CBG-GFP-t19). T cell counts of peripheral blood at day 7 and 14. Peripheral blood was analyzed for T cell counts at day7 and day14 by FCM using counting beads. Bars represent means and SEM (n=5 for UTD group and n=7 for the other groups). Data are representative of 2 experiments from 2 donors.

FIG. 33 depicts T cell counts of peripheral blood at days 7, 14 and 28 (related with FIG. 32). Peripheral blood was analyzed for total T cell, CD4 and CD8 T cell counts by FCM. Bars represent means and SEM. Dots and bars represent mean and SEMs. Data are representative of 2 experiments from 2 donors.

FIG. 34 illustrates the finding that CCR4-CAR(KW_L2H) induces antitumor efficacy comparable to that of CD19-CAR in NSG mice engrafted with HH expressing CD19 (HH-CBG-GFP-t19). Cytokine levels of serum at day 6. Serum was collected from mouse whole blood at day 6 using the same experimental schedule as figure A, B and C. Cytokines were analyzed by high sensitivity LUMINEX assay. Bars represent means and SEM (n=4 for UTD group and 5 for the other groups.). ns, not significant; *, p<0.05; **, p<0.01; ****, p<0.0001 by one-way ANOVA with Tukey's post-hoc test.

FIG. 35 depicts phenotypes of primary CTCL cells. Primary CTCL cells were obtained from a patient with Sezary syndrome. Phenotypes of tumor cells were analyzed by FCM. Normal T cell population (CD8 positive or CD4, CD7 and CD26 positive) were very rare.

FIG. 36 illustrates the finding that CCR4-CAR T cells can lyse and respond to patient derived primary CTCL cells. Cytokine production by CCR4-CAR T cells upon the stimulation of primary CTCL cells. Cytokine production of CAR T cells at 6 hours post stimulation was analyzed by intracellular staining and FCM. Gates represent positive population of indicated cytokines in CAR positive cells. Data are representative of 2 experiments from 2 donors.

FIG. 37 illustrates the finding that CCR4-CAR T cells can lyse and respond to patient derived primary CTCL cells. Lytic activity of CCR4-CAR T cells against primary CTCL cells. Killing activity of CART cells against each cell at 4 hours of co-culture was analyzed by 51 Cr release assay. Dots and bars represent mean and SD (triplicate). Data are representative of 2 experiments from 2 donors.

FIG. 38 illustrates the CCR4CAR KW_L2H tEGFR vector map.

FIG. 39 illustrates the CCR4CAR KW_L2H CD20 vector map.

FIG. 40 illustrates the CCR4CAR KW_L2H iCasp9 vector map.

FIG. 41 illustrates the CCR4CAR KW_L2H HSVTK vector map.

FIG. 42 is a plot illustrating GCV-induced depletion of CAR T cells in seven-day culture using the HSVTK depletion system.

FIG. 43 illustrates the dnTGFbR-T2A-CCR4CAR-P2A-HSVTK vector map.

FIG. 44 illustrates the CCR4CAR-P2A-dnTGFbR-T2A-HSVTK vector map.

FIG. 45 illustrates the CCR4CAR-P2A-HSVTK-T2A-dnTGFbR vector map.

FIG. 46 is a plot illustrating cytotoxicity of CCR4CAR-HSVTK-dnTGFbRII.

FIG. 47 provides data illustrating the finding that shRNAs targeting CCR4 (shCCR4s) efficiently knock down CCR4 in CCR4-expressing T cell lines HH and ATN-1. HH cells (left panels) and ATN-1 cells (right panels) were transduced with sh-CCR4 or sh-Cre (negative control) using lentivirus. CCR4 expression was analyzed at day 2 post sh-RNA by FCM.

FIGS. 48A-48D illustrate the finding that CCR4 knock down CCR4CART cells can be efficiently produced by careful selection of sh-CCR4 target sequences. FIG. 48A provides the schedule for shCCR4 and CCR4-CAR double transduction. FIG. 48B provides data showing decreased CCR4 expression on T cells at day 3 post anti-CD3/28 stimulation (day2 post sh-RNA introduction). FIG. 48C shows CART cells expansion during CCR4 knock down CCR4-CAR T cell preparation. Sh-CCR4 #3 that provides the lowest knock down efficiency induced greater T cell expansion. FIG. 48D shows CCR4-CAR expression on days 5 and 10 post T cell stimulation. Sh-CCR4 #3 that has the lowest CCR4 knock down efficiency induced greater T cell expansion and CAR transduction efficiency.

FIGS. 49A-49B illustrate the finding that CCR4-CART cells can cure mogamulizumab-resistant tumors. FIG. 49A is a schematic representation of the mogamulizumab-resistant HH tumor xenograft mouse model. FIG. 49B provides data illustrating the finding that CCR4-CAR T cells can cure mogamulizumab refractory HH tumors.

FIGS. 50A-50B illustrate the finding that CCR4-CAR T cells can be established efficiently and safely from patient derived PBMCs. FIG. 50A shows CCR4-CAR and CD19-CAR expression on T cells at days 7, 14 and 24 post T cell stimulation. FIG. 50B shows a significant decrease of contaminated ATLL cells in CCR4-CAR T cell product but not in CD19-CAR T cell product.

### DETAILED DESCRIPTION

The present invention is based on the discovery that CCR4-CAR T cells comprising an anti-CCR4 CAR derived from mogamulizumab are highly effective for killing tumor cells, including mogamulizumab refractory tumor cells. Efficacy was shown to be associated with fratricidal depletion of Th2, Treg and Th17 subsets (all CCR4 positive) while sparing the Th1 subset (CCR4 negative).

The present invention provides compositions and methods for modified immune cells or precursors thereof (e.g., modified T cells) comprising chimeric antigen receptors (CARs) capable of binding CCR4 (anti-CCR4 CARs). Also provided are methods of using the anti-CCR4 CAR-expressing cells to treat cancer, and T cell-depleting systems for use in combination with anti-CCR4 CAR cell therapy. In one aspect, the invention provides a method of treating cancer comprising administering modified cells comprising an anti-CCR4 CAR to a subject having cancer, wherein the modified cells deplete CCR4-positive T cells and not CCR4-negative T cells in the subject, thereby treating the cancer.

Optimization of a CAR construct, in particular the scFv, may enhance CAR potency. Therefore, in one embodiment, the present disclosure identifies highly effective CCR4-CAR T cells for the treatment of T cell malignancies, and determines the impact of potential CCR4-CAR mediated fratricide of normal T cells and CAR T cell product. In other embodiments, the present disclosure provides T cell-depleting systems for use as a safety switch in combination with anti-CCR4 CAR cell therapy to shutdown CAR T cells when off-tumor toxicity is observed.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used herein, may be a human or non-human mammal. Non-human mammals include, for example, livestock and pets, such as ovine, bovine, porcine, canine, feline and murine mammals, as well as simian and non-human primate mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Chimeric Antigen Receptors

The present invention provides compositions and methods comprising chimeric antigen receptors (CARs) capable of binding CCR4. CARs of the present invention comprise an anti-CCR4 antigen binding domain, a transmembrane domain, and an intracellular domain. In certain embodiments, the anti-CCR4 antigen binding domain is an anti-CCR4 scFv. In some embodiments, the CAR comprises an anti-CCR4 scFv, a transmembrane domain, a 4-1BB costimulatory domain, and a CD3ζ signaling domain. In some embodiments, the CAR further comprises a leader sequence and a hinge domain. In preferred embodiments, the anti-CCR4 scFv has a heavy chain and a light chain derived from mogamulizumab.

Also provided are compositions and methods for modified immune cells or precursors thereof, e.g., modified T cells, comprising the CAR. Thus, in some embodiments, the immune cell has been genetically modified to express the CAR. Nucleic acids encoding the CARs, vectors comprising the nucleic acids, and modified cells (e.g. modified T cells) comprising the CARs, vectors, or nucleic acids, are also provided. In certain embodiments, the nucleic acid encodes a CAR comprising an anti-CCR4 antigen binding domain, a transmembrane domain, and an intracellular domain and further encodes a safety switch to induce CAR T cell depletion in a subject upon administration of a safety switch agent. The CAR may be linked to the safety switch via a 2A self-cleaving sequence as described herein, such as a P2A or T2A sequence. In one embodiment, the safety switch is truncated EGFR (EGFRt) and the safety switch agent is an anti-EGFR antibody such as cetuximab. In one embodiment, the safety switch is CD20 and the safety switch agent is an anti-CD20 antibody, such as rituximab. In one embodiment, the safety switch is inducible caspase 9 (iCasp9) and the safety switch agent is a dimerizing drug such as AP1903. In one embodiment, the safety switch is Herpes simplex virus-1 thymidine kinase (HSVTK) and the safety switch agent is ganciclovir (GCV).

In certain embodiments, the CAR-linked safety switch further comprises a dominant-negative TGFb receptor type II (dnTGFbRII). In some embodiments, the CAR of the CAR-linked safety switch is linked to the dnTGFbRII, for example via a 2A self-cleaving sequence as described herein, such as a P2A or T2A sequence. In some embodiments, the safety switch of the CAR-linked safety switch is linked to the dnTGFbRII, for example via a 2A self-cleaving sequence as described herein, such as a P2A or T2A sequence. In some embodiments, the CAR and the safety switch of the CAR-linked safety switch are each linked to the dnTGFbRII, for example via a 2A self-cleaving sequence as described herein, such as a P2A or T2A sequence. In some embodiments, the CAR-linked safety switch comprises an anti-CCR4 CAR, an HSVTK safety switch, and a dnTGFbRII, wherein the anti-CCR4 CAR, the HSVTK, and the dnTGFbRII may be present in any order from N-terminus to C-terminus of the CAR-linked safety switch.

The antigen binding domain of the CAR is operably linked to another domain of the CAR, such as a hinge, a transmembrane domain or an intracellular domain, each described elsewhere herein, for expression in the cell. In one embodiment, a first nucleic acid sequence encoding the antigen binding domain is operably linked to a second nucleic acid encoding a hinge and/or transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention. The CAR of the present invention may also include a leader sequence as described herein. The CAR of the present invention may also include a hinge domain as described herein. The CAR of the present invention may also include one or more spacer domains or linkers as described herein which may serve to link one domain of the CAR to the next domain.

### Antigen Binding Domain

The antigen binding domain of a CAR is an extracellular region of the CAR for binding to a specific target antigen including proteins, carbohydrates, and glycolipids. The CAR of the invention comprises an antigen binding domain that is capable of binding CCR4.

The antigen binding domain can include any domain that binds to the antigen and may include, but is not limited to, a monoclonal antibody (mAb), a polyclonal antibody, a synthetic antibody, a human antibody, a humanized antibody, a non-human antibody, a single-domain antibody, a full length antibody or any antigen-binding fragment thereof, a Fab, and a single-chain variable fragment (scFv). In some embodiments, the antigen binding domain comprises an aglycosylated antibody or a fragment thereof or scFv thereof. In certain embodiments, the antigen binding domain comprises an afucosylated humanized anti-CCR4 mAb. In certain embodiments, the antigen binding domain is an scFv having a heavy chain and a light chain derived from mogamulizumab.

As used herein, the term “single-chain variable fragment” or “scFv” is a fusion protein of the variable regions of the heavy (VH) and light (VL) chains of an immunoglobulin (e.g., mouse or human) covalently linked to form a VH::VL heterodimer. The variable heavy (VH) and light (VL) chains are either joined directly or joined by a peptide linker, which connects the N-terminus of the VH with the C-terminus of the VL, or the C-terminus of the VH with the N-terminus of the VL. In some embodiments, the antigen binding domain (e.g., CCR4 binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (SG)ₙ, (GSGGS)ₙ (SEQ ID NO:115), (GGGS)n (SEQ ID NO:116), and (GGGGS)ₙ (SEQ ID NO:117), where n represents an integer of at least 1. Exemplary linker sequences can comprise amino acid sequences including, without limitation, GGSG (SEQ ID NO:118), GGSGG (SEQ ID NO:119), GSGSG (SEQ ID NO:120), GSGGG (SEQ ID NO:121), GGGSG (SEQ ID NO:122), GSSSG (SEQ ID NO:123), GGGGS (SEQ ID NO:124), GGGGSGGGGSGGGGSGGGGS (SEQ ID NO:125) and the like. Those of skill in the art would be able to select the appropriate linker sequence for use in the present invention. In one embodiment, an antigen binding domain of the present invention comprises a heavy chain variable region (VH) and a light chain variable region (VL), wherein the VH and VL is separated by the linker sequence having the amino acid sequence GGGGSGGGGSGGGGSGGGGS (SEQ ID NO:125), which may be encoded by the nucleic acid sequence

(SEQ ID NO: 126)
GGAGGAGGTGGATCAGGTGGAGGTGGAAGCGGGGG
AGGAGGTTCCGGCGGCGGAGGATCA.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof, or a humanized murine antibody or a fragment thereof.

In certain embodiments, the antigen binding domain comprises a heavy chain variable region that comprises three heavy chain complementarity determining regions (HCDRs) and a light chain variable region that comprises three light chain complementarity determining regions (LCDRs). In certain embodiments, HCDR1 comprises the amino acid sequence of SEQ ID NO: 2, and/or HCDR2 comprises the amino acid sequence of SEQ ID NO: 4, and/or HCDR3 comprises the amino acid sequence of SEQ ID NO: 6, and/or LCDR1 comprises the amino acid sequence of SEQ ID NO: 8, and/or LCDR2 comprises the amino acid sequence of SEQ ID NO: 10, and/or LCDR3 comprises the amino acid sequence of SEQ ID NO: 12.

In certain embodiments, the heavy chain variable region (VH) of the antigen binding domain comprises an amino acid sequence at least 80%, 85%, 90%, 95%, 96%, 97%, 98%, 99%, or 100% identical to SEQ ID NO: 112, and/or the light chain variable region (VL) comprises an amino acid sequence at least 80%, 85%, 90%, 95%, 96%, 97%, 98%, 99%, or 100% identical to SEQ ID NO: 114.

In certain embodiments, the antigen binding domain is a single-chain variable fragment (scFv) comprising an amino acid sequence at least 80%, 85%, 90%, 95%, 96%, 97%, 98%, 99%, or 100% identical to SEQ ID NO: 16.

In certain embodiments, the antigen binding domain comprises a linker. In certain embodiments, the linker comprises SEQ ID NO: 125.

Tolerable variations of the antigen binding domain sequences will be known to those of skill in the art. For example, in some embodiments the antigen binding domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences set forth in any one of SEQ ID NOs: 2, 4, 6, 8, 10, 12, 16, 112, 114, and 125.

### Transmembrane Domain

CARs of the present invention may comprise a transmembrane domain that connects the antigen binding domain of the CAR to the intracellular domain of the CAR. The transmembrane domain of the CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), ICOS, CD278, Toll-like receptor 1 (TLR1), TLR2, TLR3, TLR4, TLR5, TLR6, TLR7, TLR8, TLR9 or a transmembrane domain derived from a killer immunoglobulin-like receptor (KIR).

In certain embodiments, the transmembrane domain comprises a transmembrane domain of CD8. In certain embodiments, the transmembrane domain of CD8 is a transmembrane domain of CD8α. In certain embodiments, the transmembrane domain comprises the amino acid sequence set forth in SEQ ID NO: 36.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in the CAR.

In some embodiments, the transmembrane domain further comprises a hinge region. The CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, the CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region). In certain embodiments, the hinge region is a CD8a hinge. In certain embodiments, the hinge region comprises the amino acid sequence set forth in SEQ ID NO: 34.

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers (including, for example, (GS)ₙ, (GSGGS)ₙ (SEQ ID NO:115) and (GGGS)ₙ (SEQ ID NO:116), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, (GGGGS)ₙ (SEQ ID NO:117), GGSG (SEQ ID NO:118), GGSGG (SEQ ID NO:119), GSGSG (SEQ ID NO:120), GSGGG (SEQ ID NO:121), GGGSG (SEQ ID NO:122), GSSSG (SEQ ID NO:123), GGGGS (SEQ ID NO:124) and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO:132); CPPC (SEQ ID NO:133); CPEPKSCDTPPPCPR (SEQ ID NO:134) (see, e.g., Glaser et al., J. Biol. Chem. (2005) 280:41494-41503); ELKTPLGDTTHT (SEQ ID NO:135); KSCDKTHTCP (SEQ ID NO:136); KCCVDCP (SEQ ID NO:137); KYGPPCP (SEQ ID NO:138); EPKSCDKTHTCPPCP (SEQ ID NO:139) (human IgG1 hinge); ERKCCVECPPCP (SEQ ID NO:140) (human IgG2 hinge); ELKTPLGDTTHTCPRCP (SEQ ID NO:141) (human IgG3 hinge); SPNMVPHAHHAQ (SEQ ID NO:142) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 143); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

The CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in the CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of the CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular domains described herein can be combined with any of the antigen binding domains described herein, any of the transmembrane domains described herein, or any of the other domains described herein that may be included in the CAR.

In certain embodiments, the intracellular domain comprises a costimulatory domain of 4-1BB. In certain embodiments, the costimulatory domain of 4-1BB comprises the amino acid sequence set forth in SEQ ID NO: 38. In certain embodiments, the intracellular domain comprises an intracellular domain of CD3 or a variant thereof. In certain embodiments, the intracellular domain of CD3ζ comprises the amino acid sequence set forth in SEQ ID NO: 40. In certain embodiments, the intracellular domain comprises 4-1BB and CD3. In certain embodiments, the intracellular domain comprises the amino acid sequences set forth in SEQ ID NO: 38 and SEQ ID NO: 40.

Tolerable variations of the individual CAR domain sequences (leader, antigen binding domain, hinge, transmembrane, and intracellular domains) will be known to those of skill in the art. For example, in certain embodiments the CAR domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 16, 32, 34, 36, 38, and 40.

In one aspect, the invention provides a chimeric antigen receptor (CAR) comprising an anti-CCR4 antigen binding domain, a transmembrane domain, and an intracellular domain. In certain embodiments, the CAR comprises the amino acid sequence set forth in any one of SEQ ID NOs: 30, 54, 62, 70, 82, 90, and 102. In some embodiments, the CAR is linked to a safety switch via a self-cleavable linker and the CAR-linked safety switch comprises any one of the amino acid sequences set forth in SEQ ID NOs: 42, 52, 60, 68, 76, 88, and 100.

Tolerable variations of the CAR sequences and the CAR-linked safety switch sequences will be known to those of skill in the art. For example, in certain embodiments the CAR comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in any one of SEQ ID NOs: 30, 54, 62, 70, 82, 90, and 102. In certain embodiments the CAR-linked safety switch comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences set forth in SEQ ID NOs: 42, 52, 60, 68, 76, 88, and 100.

TABLE 1
Nucleotide and amino acid sequences depletion system)
A. CCR4 CARs (without depletion system)

| | |
|---|---|
| SEQ ID NO: 1 | CCR4(KW_L2H) HCDR1-nucleotide |

aactacggca tgagc 15

| | |
|---|---|
| SEQ ID NO: 2 | CCR4(KW_L2H) HCDR1-amino acid |

NYGMS 5

| | |
|---|---|
| SEQ ID NO: 3 | CCR4(KW_L2H) HCDR2-nucleotide |

acaatcagca gcgccagcac ctacagctac taccccgata gcgtgaaggg c 51

| | |
|---|---|
| SEQ ID NO: 4 | CCR4(KW_L2H) HCDR2-amino acid |

TISSASTYSY YPDSVKG 17

| | |
|---|---|
| SEQ ID NO: 5 | CCR4(KW_L2H) HCDR3-nucleotide |

cacagcgacg gcaacttcgc ctttggctat 30

| | |
|---|---|
| SEQ ID NO: 6 | CCR4(KW_L2H) HCDR3-amino acid |

HSDGNFAFGY 10

| | |
|---|---|
| SEQ ID NO: 7 | CCR4(KW_L2H) LCDR1-nucleotide |

agatccagcc ggaacatcgt gcacatcaac ggcgacacct acctggaa 48

| | |
|---|---|
| SEQ ID NO: 8 | CCR4(KW_L2H) LCDR1-amino acid |

RSSRNIVHIN GDTYLE 16

| | |
|---|---|
| SEQ ID NO: 9 | CCR4(KW_L2H) LCDR2-nucleotide |

aaggtgtcca accggttcag c 21

| | |
|---|---|
| SEQ ID NO: 10 | CCR4(KW_L2H) LCDR2-amino acid |

KVSNRFS 7

| | |
|---|---|
| SEQ ID NO: 11 | CCR4(KW_L2H) LCDR3-nucleotide |

ttccaaggct ctctgctgcc ctggaca 27

| | |
|---|---|
| SEQ ID NO: 12 | CCR4(KW_L2H) LCDR3-amino acid |

FQGSLLPWT 9

| | |
|---|---|
| SEQ ID NO: 111 | CCR4(KW_L2H) VH chain-nucleotide |

gaagttcagc tggttgagtc tggcggcgac ctggttcagc ctggcagatc tctgagactg 60
agctgtgccg ccagcggctt catcttcagc aactacggca tgagctgggt ccgacaggcc 120
cctggaaaag gactggaatg ggtcgccaca atcagcagcg ccagcaccta cagctactac 180
cccgatagcg tgaagggcag attcaccatc agccgggaca acgccaagaa cagcctgtac 240
ctgcagatga actccctgcg cgtggaagat acagccctgt actattgcgg cagacacagc 300
gacggcaact tcgcctttgg ctattggggc cagggaaccc tggtcaccgt ttctagt 357

| | |
|---|---|
| SEQ ID NO: 112 | CCR4(KW_L2H) VH chain-amino acid |

EVQLVESGGD LVQPGRSLRL SCAASGFIFS NYGMSWVRQA PGKGLEWVAT ISSASTYSYY 60
PDSVKGRFTI SRDNAKNSLY LQMNSLRVED TALYYCGRHS DGNFAFGYWG QGTLVTVSS 119

| | |
|---|---|
| SEQ ID NO: 113 | CCR4(KW_L2H) VL chain-nucleotide |

gacgtgctga tgacacagag ccctctgagc ctgcctgtga cacctggcga acctgccagc 60
atcagctgca gatccagccg gaacatcgtg cacatcaacg gcgacaccta cctggaatgg 120
tatctgcaga agcccggcca gtctcctcag ctgctgatct acaaggtgtc caaccggttc 180
agcggcgtgc ccgatagatt ttctggcagc ggctctggca ccgacttcac cctgaagatc 240
tccagagtgg aagccgagga cgtgggcgtg tactactgct tccaaggctc tctgctgccc 300
tggacatttg gccagggcac caaggtggaa atcaag 336

| | |
|---|---|
| SEQ ID NO: 114 | CCR4(KW_L2H) VL chain-amino acid |

DVLMTQSPLS LPVTPGEPAS ISCRSSRNIV HINGDTYLEW YLQKPGQSPQ LLIYKVSNRF 60
SGVPDRFSGS GSGTDFTLKI SRVEAEDVGV YYCFQGSLLP WTFGQGTKVE IK 112

| | |
|---|---|
| SEQ ID NO: 13 | CCR4(KW_H2L) scFv-nucleotide |

gaagtgcagc tggttgagtc tggcggagat ctggtgcagc ctggcagaag cctgagactg 60
tcttgtgccg ccagcggctt catcttcagc aactacggca tgagctgggt ccgacaggcc 120
cctggaaaag gccttgaatg ggtcgccaca atcagcagcg ccagcaccta cagctactac 180
cccgatagcg tgaagggcag attcaccatc agccgggaca acgccaagaa cagcctgtac 240
ctgcagatga actccctgcg cgtggaagat acagccctgt actactgcgg cagacacagc 300
gacggcaatt tcgcctttgg ctattggggc cagggcaccc tggttaccgt ttctagcgga 360
ggaggtggat caggtggagg tggaagcggg ggaggaggtt ccggcggcgg aggatcagat 420
gttctgatga cacagagccc tctgagcctg cctgtgacac ctggcgaacc tgccagcatc 480
agctgcagat ccagccggaa catcgtgcac atcaacggcg acacctacct ggaatggtat 540
ctgcagaagc ccggccagtc tcctcagctg ctgatctaca aggtgtccaa ccggttcagc 600
ggcgtgcccg atagattttc tggcagcggc tctggcaccg acttcaccct gaagatctcc 660
agagtggaag ccgaggacgt gggcgtctac tactgcttcc aaggctctct gctgccctgg 720
acatttggac agggcaccaa ggtggaaatc aag 753

| | |
|---|---|
| SEQ ID NO: 14 | CCR4(KW H2L) scFv-amino acid |

EVQLVESGGD LVQPGRSLRL SCAASGFIFS NYGMSWVRQA PGKGLEWVAT ISSASTYSYY 60
PDSVKGRFTI SRDNAKNSLY LQMNSLRVED TALYYCGRHS DGNFAFGYWG QGTLVTVSSG 120
GGGSGGGGSG GGGSGGGGSD VLMTQSPLSL PVTPGEPASI SCRSSRNIVH INGDTYLEWY 180
LQKPGQSPQL LIYKVSNRFS GVPDRESGSG SGTDFTLKIS RVEAEDVGVY YCFQGSLLPW 240
TFGQGTKVEI K 251

| | |
|---|---|
| SEQ ID NO: 15 | CCR4(KW_L2H) scFv-nucleotide |

gacgtgctga tgacacagag ccctctgagc ctgcctgtga cacctggcga acctgccagc 60
atcagctgca gatccagccg gaacatcgtg cacatcaacg gcgacaccta cctggaatgg 120
tatctgcaga agcccggcca gtctcctcag ctgctgatct acaaggtgtc caaccggttc 180
agcggcgtgc ccgatagatt ttctggcagc ggctctggca ccgacttcac cctgaagatc 240
tccagagtgg aagccgagga cgtgggcgtg tactactgct tccaaggctc tctgctgccc 300
tggacatttg gccagggcac caaggtggaa atcaagggag gaggtggatc aggtggaggt 360
ggaagcgggg gaggaggttc cggcggcgga ggatcagaag ttcagctggt tgagtctggc 420
ggcgacctgg ttcagcctgg cagatctctg agactgagct gtgccgccag cggcttcatc 480
ttcagcaact acggcatgag ctgggtccga caggcccctg gaaaaggact ggaatgggtc 540
gccacaatca gcagcgccag cacctacagc tactaccccg atagcgtgaa gggcagattc 600
accatcagcc gggacaacgc caagaacagc ctgtacctgc agatgaactc cctgcgcgtg 660
gaagatacag ccctgtacta ttgcggcaga cacagcgacg gcaacttcgc ctttggctat 720
tggggccagg gaaccctggt caccgtttct agt 753

| | |
|---|---|
| SEQ ID NO: 16 | CCR4(KW_L2H) scFv-amino acid |

DVLMTQSPLS LPVTPGEPAS ISCRSSRNIV HINGDTYLEW YLQKPGQSPQ LLIYKVSNRF 60
SGVPDRFSGS GSGTDFTLKI SRVEAEDVGV YYCFQGSLLP WTFGQGTKVE IKGGGGSGGG 120
GSGGGGSGGG GSEVQLVESG GDLVQPGRSL RLSCAASGFI FSNYGMSWVR QAPGKGLEWV 180
ATISSASTYS YYPDSVKGRF TISRDNAKNS LYLQMNSLRV EDTALYYCGR HSDGNFAFGY 240
WGQGTLVTVS S 251

| | |
|---|---|
| SEQ ID NO: 17 | CCR4(1-49_H2L) scFv-nucleotide |

caggtgcagc tggtgcagag cggagccgag gtgaagaagc ctggagcttc cgtcaaggtg 60
tcctgcaagg ccagcggcta caccttcgcc agcagctgga tgcactggat gcggcaggca 120
cctggacagg gcctcgaatg gatcggctgg atcaaccccg gcaacgtgaa caccaagtac 180
aacgagaagt tcaagggcag ggccaccctg accgtggaca ccagcaccaa caccgcctac 240
atggaactga gcagcctgcg gagcgaggac accgccgtgt actactgcgc cagaagcacg 300
tggtatcggc cgaatgacta ctggggccag ggcaccctgg tgaccgtgag cagcggagga 360
ggtggatcag gtggaggtgg aagcggggga ggaggttccg gcggcggagg atcagacatc 420
gtgatgaccc agagccccga cagcctggcc gtgagcctgg gcgagcgggc caccatcaac 480
tgcaagagca gccagagcat cctgtacagc agcaaccaga agaactacct ggcctggtat 540
cagcagaagc ccggccagag ccccaagctg ctgatctact gggccagcac ccgggagagc 600
ggcgtgcccg accggtttag cggcagcggc tccggcaccg acttcaccct gaccatcagc 660
agcctgcagg ccgaggacgt ggccgtgtac tactgccacc agtacaaaag cagctacacc 720
ttcggccagg gcacaaagct ggaaatcaag 750

| | |
|---|---|
| SEQ ID NO: 18 | CCR4(1-49_H2L) scFv-amino acid |

QVQLVQSGAE VKKPGASVKV SCKASGYTFA SSWMHWMRQA PGQGLEWIGW INPGNVNTKY 60
NEKFKGRATL TVDTSTNTAY MELSSLRSED TAVYYCARST WYRPNDYWGQ GTLVTVSSGG 120
GGSGGGGSGG GGSGGGGSDI VMTQSPDSLA VSLGERATIN CKSSQSILYS SNQKNYLAWY 180
QQKPGQSPKL LIYWASTRES GVPDRESGSG SGTDFTLTIS SLQAEDVAVY YCHQYKSSYT 240
FGQGTKLEIK 250

| | |
|---|---|
| SEQ ID NO: 19 | CCR4(1-49_L2H) scFv-nucleotide |

gacatcgtga tgacccagag ccccgacage ctggccgtga gcctgggcga gcgggccacc 60
atcaactgca agagcagcca gagcatcctg tacagcagca accagaagaa ctacctggcc 120
tggtatcagc agaagcccgg ccagagcccc aagctgctga tctactgggc cagcacccgg 180
gagagcggcg tgcccgaccg gtttagcggc agcggctccg gcaccgactt caccctgacc 240
atcagcagcc tgcaggccga ggacgtggcc gtgtactact gccaccagta caaaagcagc 300
tacaccttcg gccagggcac aaagctggaa atcaagggag gaggtggatc aggtggaggt 360
ggaagcgggg gaggaggttc cggcggcgga ggatcacagg tgcagctggt gcagagcgga 420
gccgaggtga agaagcctgg agcttccgtc aaggtgtcct gcaaggccag cggctacacc 480
ttcgccagca gctggatgca ctggatgcgg caggcacctg gacagggcct cgaatggatc 540
ggctggatca accccggcaa cgtgaacacc aagtacaacg agaagttcaa gggcagggcc 600
accctgaccg tggacaccag caccaacacc gcctacatgg aactgagcag cctgcggagc 660
gaggacaccg ccgtgtacta ctgcgccaga agcacgtggt atcggccgaa tgactactgg 720
ggccagggca ccctggtgac cgtgagcagc 750

| | |
|---|---|
| SEQ ID NO: 20 | CCR4(1-49_L2H) scFv-amino acid |

DIVMTQSPDS LAVSLGERAT INCKSSQSIL YSSNQKNYLA WYQQKPGQSP KLLIYWASTR 60
ESGVPDRFSG SGSGTDFTLT ISSLQAEDVA VYYCHQYKSS YTFGQGTKLE IKGGGGSGGG 120
GSGGGGSGGG GSQVQLVQSG AEVKKPGASV KVSCKASGYT FASSWMHWMR QAPGQGLEWI 180
GWINPGNVNT KYNEKFKGRA TLTVDTSTNT AYMELSSLRS EDTAVYYCAR STWYRPNDYW 240
GQGTLVTVSS 250

| | |
|---|---|
| SEQ ID NO: 21 | CCR4(2-3 H2L) scFv-nucleotide |

caggtgcagc tggtgcagag cggagccgag gtgaagaagc ctggagettc cgtcaaggtg 60
tcctgcaagg ccagcggcta caccttcgcc agcgcgtgga tgcactggat gcggcaggca 120
cctggacagg gcctcgaatg gatcggctgg atcaaccccg gcaacgtgaa caccaagtac 180
aacgagaagt tcaagggcag ggccaccctg accgtggaca ccagcaccaa caccgcctac 240
atggaactga gcagcctgcg gagcgaggac accgccgtgt actactgcgc cagaagcacg 300
tattaccggc cgctggacta ctggggccag ggcaccctgg tgaccgtgag cagcggagga 360
ggtggatcag gtggaggtgg aagcggggga ggaggttccg gcggcggagg atcagacatc 420
gtgatgaccc agagccccga cagcctggcc gtgagcctgg gcgagcgggc caccatcaac 480
tgcaagagca gccagagcat cctgtacagc agcaaccaga agaactacct ggcctggtat 540
cagcagaagc ccggccagag ccccaagctg ctgatctact gggccagcac ccgggagagc 600
ggcgtgcccg accggtttag cggcagcggc tccggcaccg acttcaccct gaccatcagc 660
agcctgcagg ccgaggacgt ggccgtgtac tactgccacc agtacatgag cagctacacc 720
ttcggccagg gcacaaagct ggaaatcaag 750

| | |
|---|---|
| SEQ ID NO: 22 | CCR4(2-3_H2L) scFv-amino acid |

QVQLVQSGAE VKKPGASVKV SCKASGYTFA SAWMHWMRQA PGQGLEWIGW INPGNVNTKY 60
NEKFKGRATL TVDTSTNTAY MELSSLRSED TAVYYCARST YYRPLDYWGQ GTLVTVSSGG 120
GGSGGGGSGG GGSGGGGSDI VMTQSPDSLA VSLGERATIN CKSSQSILYS SNQKNYLAWY 180
QQKPGQSPKL LIYWASTRES GVPDRESGSG SGTDFTLTIS SLQAEDVAVY YCHQYMSSYT 240
FGQGTKLEIK 250

| | |
|---|---|
| SEQ ID NO: 23 | CCR4(2-3 L2H) scFv-nucleotide |

gacatcgtga tgacccagag ccccgacagc ctggccgtga gcctgggcga gcgggccacc 60
atcaactgca agagcagcca gagcatcctg tacagcagca accagaagaa ctacctggcc 120
tggtatcagc agaagcccgg ccagagcccc aagctgctga tctactgggc cagcacccgg 180
gagagcggcg tgcccgaccg gtttagcggc agcggctccg gcaccgactt caccctgacc 240
atcagcagcc tgcaggccga ggacgtggcc gtgtactact gccaccagta catgagcagc 300
tacaccttcg gccagggcac aaagctggaa atcaagggag gaggtggatc aggtggaggt 360
ggaagcgggg gaggaggttc cggcggcgga ggatcacagg tgcagctggt gcagagcgga 420
gccgaggtga agaagcctgg agcttccgtc aaggtgtcct gcaaggccag cggctacacc 480
ttcgccagcg cgtggatgca ctggatgcgg caggcacctg gacagggcct cgaatggatc 540
ggctggatca accccggcaa cgtgaacacc aagtacaacg agaagttcaa gggcagggcc 600
accctgaccg tggacaccag caccaacacc gcctacatgg aactgagcag cctgcggagc 660
gaggacaccg ccgtgtacta ctgcgccaga agcacgtatt accggccgct ggactactgg 720
ggccagggca ccctggtgac cgtgagcagc 750

| | |
|---|---|
| SEQ ID NO: 24 | CCR4(2-3_L2H) scFv-amino acid |

DIVMTQSPDS LAVSLGERAT INCKSSQSIL YSSNQKNYLA WYQQKPGQSP KLLIYWASTR 60
ESGVPDRFSG SGSGTDFTLT ISSLQAEDVA VYYCHQYMSS YTFGQGTKLE IKGGGGSGGG 120
GSGGGGSGGG GSQVQLVQSG AEVKKPGASV KVSCKASGYT FASAWMHWMR QAPGQGLEWI 180
GWINPGNVNT KYNEKFKGRA TLTVDTSTNT AYMELSSLRS EDTAVYYCAR STYYRPLDYW 240
GQGTLVTVSS 250

| | |
|---|---|
| SEQ ID NO: 25 | CCR4(h1567 H2L) scFv-nucleotide |

caggtgcagc tggtgcagag cggagccgag gtgaagaagc ctggagcttc cgtcaaggtg 60
tcctgcaagg ccagcggcta caccttcgcc agctactaca tgcactggat gcggcaggca 120
cctggacagg gcctcgaatg gatcggctgg atcaaccccg gcaacgtgaa caccaagtac 180
aacgagaagt tcaagggcag ggccaccctg accgtggaca ccagcaccaa caccgcctac 240
atggaactga gcagcctgcg gagcgaggac accgccgtgt actactgcgc cagaagcacc 300
tactaccggc ccctggacta ctggggccag ggcaccctgg tgaccgtgag cagcggagga 360
ggtggatcag gtggaggtgg aagcggggga ggaggttccg gcggcggagg atcagacatc 420
gtgatgaccc agagccccga cagcctggcc gtgagcctgg gcgagcgggc caccatcaac 480
tgcaagagca gccagagcat cctgtacagc agcaaccaga agaactacct ggcctggtat 540
cagcagaagc ccggccagag ccccaagctg ctgatctact gggccagcac ccgggagagc 600
ggcgtgcccg accggtttag cggcagcggc tccggcaccg acttcaccct gaccatcagc 660
agcctgcagg ccgaggacgt ggccgtgtac tactgccacc agtacctgag cagctacacc 720
ttcggccagg gcacaaagct ggaaatcaag 750

| | |
|---|---|
| SEQ ID NO: 26 | CCR4(h1567_H2L) scFv-amino acid |

QVQLVQSGAE VKKPGASVKV SCKASGYTFA SYYMHWMRQA PGQGLEWIGW INPGNVNTKY 60
NEKFKGRATL TVDTSTNTAY MELSSLRSED TAVYYCARST YYRPLDYWGQ GTLVTVSSGG 120
GGSGGGGSGG GGSGGGGSDI VMTQSPDSLA VSLGERATIN CKSSQSILYS SNQKNYLAWY 180
QQKPGQSPKL LIYWASTRES GVPDRESGSG SGTDFTLTIS SLQAEDVAVY YCHQYLSSYT 240
FGQGTKLEIK 250

| | |
|---|---|
| SEQ ID NO: 27 | CCR4(h1567_L2H) scFv-nucleotide |

gacatcgtga tgacccagag ccccgacagc ctggccgtga gcctgggcga gcgggccacc 60
atcaactgca agagcagcca gagcatcctg tacagcagca accagaagaa ctacctggcc 120
tggtatcagc agaagcccgg ccagagcccc aagctgctga tctactgggc cagcacccgg 180
gagagcggcg tgcccgaccg gtttagcggc agcggctccg gcaccgactt caccctgacc 240
atcagcagcc tgcaggccga ggacgtggcc gtgtactact gccaccagta cctgagcagc 300
tacaccttcg gccagggcac aaagctggaa atcaagggag gaggtggatc aggtggaggt 360
ggaagcgggg gaggaggttc cggcggcgga ggatcacagg tgcagctggt gcagagcgga 420
gccgaggtga agaagcctgg agcttccgtc aaggtgtcct gcaaggccag cggctacacc 480
ttcgccagct actacatgca ctggatgcgg caggcacctg gacagggcct cgaatggatc 540
ggctggatca accccggcaa cgtgaacacc aagtacaacg agaagttcaa gggcagggcc 600
accctgaccg tggacaccag caccaacacc gcctacatgg aactgagcag cctgcggagc 660
gaggacaccg ccgtgtacta ctgcgccaga agcacctact accggcccct ggactactgg 720
ggccagggca ccctggtgac cgtgagcagc 750

| | |
|---|---|
| SEQ ID NO: 28 | CCR4(h1567_L2H) scFv-amino acid |

DIVMTQSPDS LAVSLGERAT INCKSSQSIL YSSNQKNYLA WYQQKPGQSP KLLIYWASTR 60
ESGVPDRFSG SGSGTDFTLT ISSLQAEDVA VYYCHQYLSS YTFGQGTKLE IKGGGGSGGG 120
GSGGGGSGGG GSQVQLVQSG AEVKKPGASV KVSCKASGYT FASYYMHWMR QAPGQGLEWI 180
GWINPGNVNT KYNEKFKGRA TLTVDTSTNT AYMELSSLRS EDTAVYYCAR STYYRPLDYW 240
GQGTLVTVSS 250

| | |
|---|---|
| SEQ ID NO: 29 | CCR4(KW L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgctaa 1500

| | |
|---|---|
| SEQ ID NO: 30 | CCR4(KW_L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPR 499

| | |
|---|---|
| SEQ ID NO: 31 | CD8 leader-nucleotide |

atggccctgc ctgtgacage cctgctgctg cctctggctc tgctgctgca tgccgctaga 60
CCC 63

| | |
|---|---|
| SEQ ID NO: 32 | CD8 leader-amino acid |

MALPVTALLL PLALLLHAAR P 21

| | |
|---|---|
| SEQ ID NO: 33 | CD8 hinge-nucleotide |

accacgacgc cagcgcegcg accaccaaca ccggcgccca ccatcgcgtc gcagcccctg 60
tccctgcgcc cagaggcgtg ccggccagcg gcggggggcg cagtgcacac gagggggctg 120
gacttcgcct gtgat 135

| | |
|---|---|
| SEQ ID NO: 34 | CD8 hinge-amino acid |

TTTPAPRPPT PAPTIASQPL SLRPEACRPA AGGAVHTRGL DFACD 45

| | |
|---|---|
| SEQ ID NO: 35 | CD8 transmembrane-nucleotide |

atctacatct gggcgccctt ggccgggact tgtggggtcc ttctcctgtc actggttatc 60
accctttact gc 72

| | |
|---|---|
| SEQ ID NO: 36 | CD8 transmembrane-amino acid |

IYIWAPLAGT CGVLLLSLVI TLYC 24

| | |
|---|---|
| SEQ ID NO: 37 | 4-1BB intracellular domain-nucleotide |

aaacggggca gaaagaaact cctgtatata ttcaaacaac catttatgag accagtacaa 60
actactcaag aggaagatgg ctgtagctgc cgatttccag aagaagaaga aggaggatgt 120
gaactg 126

| | |
|---|---|
| SEQ ID NO: 38 | 4-1BB intracellular domain-amino acid |

KRGRKKLLYI FKQPFMRPVQ TTQEEDGCSC RFPEEEEGGC EL 42

| | |
|---|---|
| SEQ ID NO: 39 | CD3 zeta-nucleotide |

agagtgaagt tcagcaggag cgcagacgcc cccgcgtaca agcagggcca gaaccagctc 60
tataacgagc tcaatctagg acgaagagag gagtacgatg ttttggacaa gagacgtggc 120
cgggaccctg agatgggggg aaagccgaga aggaagaacc ctcaggaagg cctgtacaat 180
gaactgcaga aagataagat ggcggaggcc tacagtgaga ttgggatgaa aggcgagcgc 240
cggaggggca aggggcacga tggcctttac cagggtctca gtacagccac caaggacacc 300
tacgacgccc ttcacatgca ggccctgccc cctcgctaa 339

| | |
|---|---|
| SEQ ID NO: 40 | CD3 zeta-amino acid |

RVKFSRSADA PAYKQGONQL YNELNLGRRE EYDVLDKRRG RDPEMGGKPR RKNPQEGLYN 60
ELQKDKMAEA YSEIGMKGER RRGKGHDGLY QGLSTATKDT YDALHMQALP PR 112
B. CCR4(KW_L2H)-truncated EGFR (tEGFR) depletion system

| | |
|---|---|
| SEQ ID NO: 41 | CCR4(KW_L2H)-tEGFR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgccat 1500
atgggaagcg gagctactaa cttcagcctg ctgaagcagg ctggagacgt ggaggagaac 1560
cctggaccta tgcttctcct ggtgacaagc cttctgctct gtgagttacc acacccagca 1620
ttcctcctga tcccacgcaa agtgtgtaac ggaataggta ttggtgaatt taaagactca 1680
ctctccataa atgctacgaa tattaaacac ttcaaaaact gcacctccat cagtggcgat 1740
ctccacatcc tgccggtggc atttaggggt gactccttca cacatactcc tcctctggac 1800
ccacaggaac tggatattct gaaaaccgta aaggaaatca cagggttttt gctgattcag 1860
gcttggcctg aaaacaggac ggacctccat gcctttgaga acctagaaat catacgcggc 1920
aggaccaagc aacatggtca gttttctctt gcagtcgtca gcctgaacat aacatccttg 1980
ggattacgct ccctcaagga gataagtgat ggagatgtga taatttcagg aaacaaaaat 2040
ttgtgctatg caaatacaat aaactggaaa aaactgtttg ggacctccgg tcagaaaacc 2100
aaaattataa gcaacagagg tgaaaacagc tgcaaggcca caggccaggt ctgccatgcc 2160
ttgtgctccc ccgagggctg ctggggcccg gagcccaggg actgcgtctc ttgccggaat 2220
gtcagccgag gcagggaatg cgtggacaag tgcaaccttc tggagggtga gccaagggag 2280
tttgtggaga actctgagtg catacagtgc cacccagagt gcctgcctca ggccatgaac 2340
atcacctgca caggacgggg accagacaac tgtatccagt gtgcccacta cattgacggc 2400
ccccactgcg tcaagacctg cccggcagga gtcatgggag aaaacaacac cctggtctgg 2460
aagtacgcag acgccggcca tgtgtgccac ctgtgccatc caaactgcac ctacggatgc 2520
actgggccag gtcttgaagg ctgtccaacg aatgggccta agatcccgtc catcgccact 2580
gggatggtgg gggccctcct cttgctgctg gtggtggccc tggggatcgg cctcttcatg 2640
tag 2643

| | |
|---|---|
| SEQ ID NO: 42 | CCR4(KW_L2H)-tEGFR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRH MGSGATNFSL LKQAGDVEEN PGPMLLLVTS LLLCELPHPA 540
FLLIPRKVCN GIGIGEFKDS LSINATNIKH FKNCTSISGD LHILPVAFRG DSFTHTPPLD 600
PQELDILKTV KEITGELLIQ AWPENRTDLH AFENLEIIRG RTKQHGQFSL AVVSLNITSL 660
GLRSLKEISD GDVIISGNKN LCYANTINWK KLFGTSGQKT KIISNRGENS CKATGQVCHA 720
LCSPEGCWGP EPRDCVSCRN VSRGRECVDK CNLLEGEPRE FVENSECIQC HPECLPQAMN 780
ITCTGRGPDN CIQCAHYIDG PHCVKTCPAG VMGENNTLVW KYADAGHVCH LCHPNCTYGC 840
TGPGLEGCPT NGPKIPSIAT GMVGALLLLL VVALGIGLFM 880

| | |
|---|---|
| SEQ ID NO: 43 | CCR4(KW L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgccat 1500
atg 1503

| | |
|---|---|
| SEQ ID NO: 44 | CCR4(KW_L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRETISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRH M 501

| | |
|---|---|
| SEQ ID NO: 45 | P2A-nucleotide |

ggaagcggag ctactaactt cagcctgctg aagcaggctg gagacgtgga ggagaaccct 60
ggacct 66

| | |
|---|---|
| SEQ ID NO: 46 | P2A-amino acid |

GSGATNFSLL KQAGDVEENP GP 22

| | |
|---|---|
| SEQ ID NO: 47 | GMSCFR leader-nucleotide |

atgcttctcc tggtgacaag ccttctgctc tgtgagttac cacacccagc attcctcctg 60
atccca 66

| | |
|---|---|
| SEQ ID NO: 48 | GMSCFR leader-amino acid |

MLLLVTSLLL CELPHPAFLL IP 22

| | |
|---|---|
| SEQ ID NO: 49 | tEGFR-nucleotide |

cgcaaagtgt gtaacggaat aggtattggt gaatttaaag actcactctc cataaatgct 60
acgaatatta aacacttcaa aaactgcacc tccatcagtg gcgatctcca catcctgccg 120
gtggcattta ggggtgactc cttcacacat actcctcctc tggacccaca ggaactggat 180
attctgaaaa ccgtaaagga aatcacaggg tttttgctga ttcaggcttg gcctgaaaac 240
aggacggacc tccatgcctt tgagaaccta gaaatcatac gcggcaggac caagcaacat 300
ggtcagtttt ctcttgcagt cgtcagcctg aacataacat ccttgggatt acgctccctc 360
aaggagataa gtgatggaga tgtgataatt tcaggaaaca aaaatttgtg ctatgcaaat 420
acaataaact ggaaaaaact gtttgggacc tccggtcaga aaaccaaaat tataagcaac 480
agaggtgaaa acagctgcaa ggccacaggc caggtctgcc atgccttgtg ctcccccgag 540
ggctgctggg gcccggagcc cagggactgc gtctcttgcc ggaatgtcag ccgaggcagg 600
gaatgcgtgg acaagtgcaa ccttctggag ggtgagccaa gggagtttgt ggagaactct 660
gagtgcatac agtgccaccc agagtgcctg cctcaggcca tgaacatcac ctgcacagga 720
cggggaccag acaactgtat ccagtgtgcc cactacattg acggccccca ctgcgtcaag 780
acctgcccgg caggagtcat gggagaaaac aacaccctgg tctggaagta cgcagacgcc 840
ggccatgtgt gccacctgtg ccatccaaac tgcacctacg gatgcactgg gccaggtctt 900
gaaggctgtc caacgaatgg gcctaagatc ccgtccatcg ccactgggat ggtgggggcc 960
ctcctcttgc tgctggtggt ggccctgggg atcggcctct tcatgtag 1008

| | |
|---|---|
| SEQ ID NO: 50 | tEGFR-amino acid |

RKVCNGIGIG EFKDSLSINA TNIKHFKNCT SISGDLHILP VAFRGDSFTH TPPLDPQELD 60
ILKTVKEITG FLLIQAWPEN RTDLHAFENL EIIRGRTKQH GQFSLAVVSL NITSLGLRSL 120
KEISDGDVII SGNKNLCYAN TINWKKLFGT SGQKTKIISN RGENSCKATG QVCHALCSPE 180
GCWGPEPRDC VSCRNVSRGR ECVDKCNLLE GEPREFVENS ECIQCHPECL PQAMNITCTG 240
RGPDNCIQCA HYIDGPHCVK TCPAGVMGEN NTLVWKYADA GHVCHLCHPN CTYGCTGPGL 300
EGCPTNGPKI PSIATGMVGA LLLLLVVALG IGLFM 335
C. CCR4(KW_L2H)-CD20 depletion system

| | |
|---|---|
| SEQ ID NO: 51 | CCR4(KW_L2H)-CD20-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgccat 1500
atgggaagcg gagctactaa cttcagcctg ctgaagcagg ctggagacgt ggaggagaac 1560
cctggaccta tgacaacacc cagaaattca gtaaatggga ctttcccggc agagccaatg 1620
aaaggcccta ttgctatgca atctggtcca aaaccactct tcaggaggat gtcttcactg 1680
gtgggcccca cgcaatcctt cttcatgagg gaatctaaga ctttgggggc tgtccagatt 1740
atgaatgggc tcttccacat tgccctgggg ggtcttctga tgatcccagc agggatctat 1800
gcacccatct gtgtgactgt gtggtatcct ctctggggag gcattatgta tattatttcc 1860
ggatcactcc tggcagcaac ggagaaaaac tccaggaagt gtttggtcaa aggaaaaatg 1920
ataatgaact cattgagcct ctttgctgcc atttctggaa tgattctttc aatcatggac 1980
atacttaata ttaaaatttc ccatttttta aaaatggaga gtctgaattt tattagagct 2040
cacacaccat atattaacat atacaactgt gaaccagcta atccctctga gaaaaactcc 2100
ccatctaccc aatactgtta cagcatacaa tctctgttct tgggcatttt gtcagtgatg 2160
ctgatctttg ccttcttcca ggaacttgta atagctggca tcgttgagaa tgaatggaaa 2220
agaacgtgct ccagacccaa atctaacata gttctcctgt cagcagaaga aaaaaaagaa 2280
cagactattg aaataaaaga agaagtggtt gggctaactg aaacatcttc ccaaccaaag 2340
aatgaagaag acattgaaat tattccaatc caagaagagg aagaagaaga aacagagacg 2400
aactttccag aacctcccca agatcaggaa tcctcaccaa tagaaaatga cagctctcct 2460
taa 2463

| | |
|---|---|
| SEQ ID NO: 52 | CCR4(KW_L2H)-CD20-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRH MGSGATNESL LKQAGDVEEN PGPMTTPRNS VNGTFPAEPM 540
KGPIAMQSGP KPLFRRMSSL VGPTQSFFMR ESKTLGAVQI MNGLFHIALG GLLMIPAGIY 600
APICVTVWYP LWGGIMYIIS GSLLAATEKN SRKCLVKGKM IMNSLSLFAA ISGMILSIMD 660
ILNIKISHFL KMESLNFIRA HTPYINIYNC EPANPSEKNS PSTQYCYSIQ SLFLGILSVM 720
LIFAFFQELV IAGIVENEWK RTCSRPKSNI VLLSAEEKKE QTIEIKEEVV GLTETSSQPK 780
NEEDIEIIPI QEEEEEETET NFPEPPQDQE SSPIENDSSP 820

| | |
|---|---|
| SEQ ID NO: 53 | CCR4(KW L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgccat 1500
atg 1503

| | |
|---|---|
| SEQ ID NO: 54 | CCR4(KW_L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRH M 501

| | |
|---|---|
| SEQ ID NO: 55 | P2A-nucleotide |

ggaagcggag ctactaactt cagcctgctg aagcaggctg gagacgtgga ggagaaccct 60
ggacct 66

| | |
|---|---|
| SEQ ID NO: 56 | P2A-amino acid |

GSGATNFSLL KQAGDVEENP GP 22

| | |
|---|---|
| SEQ ID NO: 57 | CD20-nucleotide |

atgacaacac ccagaaattc agtaaatggg actttcccgg cagagccaat gaaaggccct 60
attgctatgc aatctggtcc aaaaccactc ttcaggagga tgtcttcact ggtgggcccc 120
acgcaatcct tcttcatgag ggaatctaag actttggggg ctgtccagat tatgaatggg 180
ctcttccaca ttgccctggg gggtcttctg atgatcccag cagggatcta tgcacccatc 240
tgtgtgactg tgtggtatcc tctctgggga ggcattatgt atattatttc cggatcactc 300
ctggcagcaa cggagaaaaa ctccaggaag tgtttggtca aaggaaaaat gataatgaac 360
tcattgagcc tctttgctgc catttctgga atgattcttt caatcatgga catacttaat 420
attaaaattt cccatttttt aaaaatggag agtctgaatt ttattagagc tcacacacca 480
tatattaaca tatacaactg tgaaccagct aatccctctg agaaaaactc cccatctacc 540
caatactgtt acagcataca atctctgttc ttgggcattt tgtcagtgat gctgatcttt 600
gccttcttcc aggaacttgt aatagctggc atcgttgaga atgaatggaa aagaacgtgc 660
tccagaccca aatctaacat agttctcctg tcagcagaag aaaaaaaaga acagactatt 720
gaaataaaag aagaagtggt tgggctaact gaaacatctt cccaaccaaa gaatgaagaa 780
gacattgaaa ttattccaat ccaagaagag gaagaagaag aaacagagac gaactttcca 840
gaacctcccc aagatcagga atcctcacca atagaaaatg acagctctcc ttaa 894

| | |
|---|---|
| SEQ ID NO: 58 | CD20-amino acid |

MTTPRNSVNG TFPAEPMKGP IAMQSGPKPL FRRMSSLVGP TQSFFMRESK TLGAVQIMNG 60
LFHIALGGLL MIPAGIYAPI CVTVWYPLWG GIMYIISGSL LAATEKNSRK CLVKGKMIMN 120
SLSLFAAISG MILSIMDILN IKISHFLKME SLNFIRAHTP YINIYNCEPA NPSEKNSPST 180
QYCYSIQSLF LGILSVMLIF AFFQELVIAG IVENEWKRTC SRPKSNIVLL SAEEKKEQTI 240
EIKEEVVGLT ETSSQPKNEE DIEIIPIQEE EEEETETNFP EPPQDQESSP IENDSSP 297
D. CCR4(KW_L2H)-FKBP-iCasp9 depletion system

| | |
|---|---|
| SEQ ID NO: 59 | CCR4(KW_L2H)-FKBP-iCasp9-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgccat 1500
atgggaagcg gagctactaa cttcagcctg ctgaagcagg ctggagacgt ggaggagaac 1560
cctggacctg gagtgcaggt ggagactatc tccccaggag acgggcgcac cttccccaag 1620
cgcggccaga cctgcgtggt gcactacacc gggatgcttg aagatggaaa gaaagttgat 1680
tcctcccggg acagaaacaa gccctttaag tttatgctag gcaagcagga ggtgatccga 1740
ggctgggaag aaggggttgc ccagatgagt gtgggtcaga gagccaaact gactatatct 1800
ccagattatg cctatggtgc cactgggcac ccaggcatca tcccaccaca tgccactctc 1860
gtcttcgatg tggagcttct aaaactggaa tctggcggtg gctccggatt tggtgatgtc 1920
ggtgctcttg agagtttgag gggaaatgca gatttggctt acatcctgag catggagccc 1980
tgtggccact gcctcattat caacaatgtg aacttctgcc gtgagtccgg gctccgcacc 2040
cgcactggct ccaacatcga ctgtgagaag ttgcggcgtc gcttctcctc gctgcatttc 2100
atggtggagg tgaagggcga cctgactgcc aagaaaatgg tgctggcttt gctggagctg 2160
gcgcggcagg accacggtgc tctggactgc tgcgtggtgg tcattctctc tcacggctgt 2220
caggccagcc acctgcagtt cccaggggct gtctacggca cagatggatg ccctgtgtcg 2280
gtcgagaaga ttgtgaacat cttcaatggg accagctgcc ccagcctggg agggaagccc 2340
aagctctttt tcatccaggc ctgtggtggg gagcagaaag accatgggtt tgaggtggcc 2400
tccacttccc ctgaagacga gtcccctggc agtaaccccg agccagatgc caccccgttc 2460
caggaaggtt tgaggacctt cgaccagctg gacgccatat ctagtttgcc cacacccagt 2520
gacatctttg tgtcctactc tactttccca ggttttgttt cctggaggga ccccaagagt 2580
ggctcctggt acgttgagac cctggacgac atctttgagc agtgggctca ctctgaagac 2640
ctgcagtccc tcctgcttag ggtcgctaat gctgtttcgg tgaaagggat ttataaacag 2700
atgcctggtt gctttaattt cctccggaaa aaacttttct ttaaaacatc ataa 2754

| | |
|---|---|
| SEQ ID NO: 60 | CCR4(KW_L2H)-FKBP-iCasp9-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRH MGSGATNESL LKQAGDVEEN PGPGVQVETI SPGDGRTEPK 540
RGQTCVVHYT GMLEDGKKVD SSRDRNKPFK FMLGKQEVIR GWEEGVAQMS VGQRAKLTIS 600
PDYAYGATGH PGIIPPHATL VEDVELLKLE SGGGSGFGDV GALESLRGNA DLAYILSMEP 660
CGHCLIINNV NFCRESGLRT RTGSNIDCEK LRRRFSSLHF MVEVKGDLTA KKMVLALLEL 720
ARQDHGALDC CVVVILSHGC QASHLQFPGA VYGTDGCPVS VEKIVNIFNG TSCPSLGGKP 780
KLFFIQACGG EQKDHGFEVA STSPEDESPG SNPEPDATPF QEGLRTFDQL DAISSLPTPS 840
DIFVSYSTFP GFVSWRDPKS GSWYVETLDD IFEQWAHSED LQSLLLRVAN AVSVKGIYKQ 900
MPGCFNFLRK KLFFKTS 917

| | |
|---|---|
| SEQ ID NO: 61 | CCR4(KW_L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgccat 1500
atg 1503

| | |
|---|---|
| SEQ ID NO: 62 | CCR4(KW L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRH M 501

| | |
|---|---|
| SEQ ID NO: 63 | P2A-nucleotide |

ggaagcggag ctactaactt cagcctgctg aagcaggctg gagacgtgga ggagaaccct 60
ggacct 66

| | |
|---|---|
| SEQ ID NO: 64 | P2A-amino acid |

GSGATNFSLL KQAGDVEENP GP 22

| | |
|---|---|
| SEQ ID NO: 65 | FKBP-iCasp9-nucleotide |

ggagtgcagg tggagactat ctccccagga gacgggcgca ccttccccaa gcgcggccag 60
acctgcgtgg tgcactacac cgggatgctt gaagatggaa agaaagttga ttcctcccgg 120
gacagaaaca agccctttaa gtttatgcta ggcaagcagg aggtgatccg aggctgggaa 180
gaaggggttg cccagatgag tgtgggtcag agagccaaac tgactatatc tccagattat 240
gcctatggtg ccactgggca cccaggcatc atcccaccac atgccactct cgtcttcgat 300
gtggagcttc taaaactgga atctggcggt ggctccggat ttggtgatgt cggtgctctt 360
gagagtttga ggggaaatgc agatttggct tacatcctga gcatggagcc ctgtggccac 420
tgcctcatta tcaacaatgt gaacttctgc cgtgagtccg ggctccgcac ccgcactggc 480
tccaacatcg actgtgagaa gttgcggcgt cgcttctcct cgctgcattt catggtggag 540
gtgaagggcg acctgactgc caagaaaatg gtgctggctt tgctggagct ggcgcggcag 600
gaccacggtg ctctggactg ctgcgtggtg gtcattctct ctcacggctg tcaggccagc 660
cacctgcagt tcccaggggc tgtctacggc acagatggat gccctgtgtc ggtcgagaag 720
attgtgaaca tcttcaatgg gaccagctgc cccagcctgg gagggaagcc caagctcttt 780
ttcatccagg cctgtggtgg ggagcagaaa gaccatgggt ttgaggtggc ctccacttcc 840
cctgaagacg agtcccctgg cagtaacccc gagccagatg ccaccccgtt ccaggaaggt 900
ttgaggacct tcgaccagct ggacgccata tctagtttgc ccacacccag tgacatcttt 960
gtgtcctact ctactttccc aggttttgtt tcctggaggg accccaagag tggctcctgg 1020
tacgttgaga ccctggacga catctttgag cagtgggctc actctgaaga cctgcagtcc 1080
ctcctgctta gggtcgctaa tgctgtttcg gtgaaaggga tttataaaca gatgcctggt 1140
tgctttaatt tcctccggaa aaaacttttc tttaaaacat cataa 1185

| | |
|---|---|
| SEQ ID NO: 66 | FKBP-iCasp9-amino acid |

GVQVETISPG DGRTFPKRGQ TCVVHYTGML EDGKKVDSSR DRNKPFKFML GKQEVIRGWE 60
EGVAQMSVGQ RAKLTISPDY AYGATGHPGI IPPHATLVED VELLKLESGG GSGFGDVGAL 120
ESLRGNADLA YILSMEPCGH CLIINNVNFC RESGLRTRTG SNIDCEKLRR RFSSLHEMVE 180
VKGDLTAKKM VLALLELARQ DHGALDCCVV VILSHGCQAS HLQFPGAVYG TDGCPVSVEK 240
IVNIFNGTSC PSLGGKPKLF FIQACGGEQK DHGFEVASTS PEDESPGSNP EPDATPFQEG 300
LRTFDQLDAI SSLPTPSDIF VSYSTFPGFV SWRDPKSGSW YVETLDDIFE QWAHSEDLQS 360
LLLRVANAVS VKGIYKQMPG CFNFLRKKLF FKTS 394
E. CCR4(KW_L2H)-Herpes Simplex Virus-1 Thymidine Kinase
(HSVTK) depletion system

| | |
|---|---|
| SEQ ID NO: 67 | CCR4(KW_L2H)-HSVTK-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgcgga 1500
agcggagcta ctaacttcag cctgctgaag caggctggag acgtggagga gaaccctgga 1560
cctatggctt cgtacccctg ccatcaacac gcgtctgcgt tcgaccaggc tgcgcgttct 1620
cgcggccata gcaaccgacg tacggcgttg cgccctcgcc ggcagcaaga agccacggaa 1680
gtccgcctgg agcagaaaat gcccacgcta ctgcgggttt atatagacgg tcctcacggg 1740
atggggaaaa ccaccaccac gcaactgctg gtggccctgg gttcgcgcga cgatatcgtc 1800
tacgtacccg agccgatgac ttactggcag gtgctggggg cttccgagac aatcgcgaac 1860
atctacacca cacaacaccg cctcgaccag ggcgagatat cggccgggga cgcggcggtg 1920
gtaatgacaa gcgcccagat aacaatgggc atgccttatg ccgtgaccga cgccgttctg 1980
gctcctcatg tcggggggga ggctgggagt tcacatgccc cgcccccggc cctcaccctc 2040
atcttcgacc gccatcccat cgccgccctc ctgtgctacc cggccgcgcg ataccttatg 2100
ggcagcatga ccccccaggc cgtgctggcg ttcgtggccc tcatcccgcc gaccttgccc 2160
ggcacaaaca tcgtgttggg ggcccttccg gaggacagac acatcgaccg cctggccaaa 2220
cgccagcgcc ccggcgagcg gcttgacctg gctatgctgg ccgcgattcg ccgcgtttac 2280
gggctgcttg ccaatacggt gcggtatctg cagggcggcg ggtcgtggtg ggaggattgg 2340
ggacagcttt cggggacggc cgtgccgccc cagggtgccg agccccagag caacgcgggc 2400
ccacgacccc atatcgggga cacgttattt accctgtttc gggcccccga gttgctggcc 2460
cccaacggcg acctgtataa cgtgtttgcc tgggccttgg acgtcttggc caaacgcctc 2520
cgtcccatgc acgtctttat cctggattac gaccaatcgc ccgccggctg ccgggacgcc 2580
ctgctgcaac ttacctccgg gatggtccag acccacgtca ccaccccagg ctccataccg 2640
acgatctgcg acctggcgcg cacgtttgcc cgggagatgg gggaggctaa ctaa 2694

| | |
|---|---|
| SEQ ID NO: 68 | CCR4(KW L2H)-HSVTK-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRG SGATNFSLLK QAGDVEENPG PMASYPCHQH ASAFDQAARS 540
RGHSNRRTAL RPRRQQEATE VRLEQKMPTL LRVYIDGPHG MGKTTTTQLL VALGSRDDIV 600
YVPEPMTYWQ VLGASETIAN IYTTQHRLDQ GEISAGDAAV VMTSAQITMG MPYAVTDAVL 660
APHVGGEAGS SHAPPPALTL IFDRHPIAAL LCYPAARYLM GSMTPQAVLA FVALIPPTLP 720
GTNIVLGALP EDRHIDRLAK RQRPGERLDL AMLAAIRRVY GLLANTVRYL QGGGSWWEDW 780
GQLSGTAVPP QGAEPQSNAG PRPHIGDTLF TLFRAPELLA PNGDLYNVFA WALDVLAKRL 840
RPMHVFILDY DQSPAGCRDA LLQLTSGMVQ THVTTPGSIP TICDLARTFA REMGEAN 897

| | |
|---|---|
| SEQ ID NO: 69 | CCR4(KW_L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgccat 1500
atg 1503

| | |
|---|---|
| SEQ ID NO: 70 | CCR4(KW_L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRH M 501

| | |
|---|---|
| SEQ ID NO: 71 | P2A-nucleotide |

ggaagcggag ctactaactt cagcctgctg aagcaggctg gagacgtgga ggagaaccct 60
ggacct 66

| | |
|---|---|
| SEQ ID NO: 72 | P2A-amino acid |

GSGATNFSLL KQAGDVEENP GP 22

| | |
|---|---|
| SEQ ID NO: 73 | HSVTK-nucleotide |

atggcttcgt acccctgcca tcaacacgcg tctgcgttcg accaggctgc gcgttctcgc 60
ggccatagca accgacgtac ggcgttgcgc cctcgccggc agcaagaagc cacggaagtc 120
cgcctggagc agaaaatgcc cacgctactg cgggtttata tagacggtcc tcacgggatg 180
gggaaaacca ccaccacgca actgctggtg gccctgggtt cgcgcgacga tatcgtctac 240
gtacccgagc cgatgactta ctggcaggtg ctgggggctt ccgagacaat cgcgaacatc 300
tacaccacac aacaccgcct cgaccagggc gagatatcgg ccggggacgc ggcggtggta 360
atgacaagcg cccagataac aatgggcatg ccttatgccg tgaccgacgc cgttctggct 420
cctcatgtcg ggggggaggc tgggagttca catgccccgc ccccggccct caccctcatc 480
ttcgaccgcc atcccatcgc cgccctcctg tgctacccgg ccgcgcgata ccttatgggc 540
agcatgaccc cccaggccgt gctggcgttc gtggccctca tcccgccgac cttgcccggc 600
acaaacatcg tgttgggggc ccttccggag gacagacaca tcgaccgcct ggccaaacgc 660
cagcgccccg gcgagcggct tgacctggct atgctggccg cgattcgccg cgtttacggg 720
ctgcttgcca atacggtgcg gtatctgcag ggcggcgggt cgtggtggga ggattgggga 780
cagctttcgg ggacggccgt gccgccccag ggtgccgagc cccagagcaa cgcgggccca 840
cgaccccata tcggggacac gttatttacc ctgtttcggg cccccgagtt gctggccccc 900
aacggcgacc tgtataacgt gtttgcctgg gccttggacg tcttggccaa acgcctccgt 960
cccatgcacg tctttatcct ggattacgac caatcgcccg ccggctgccg ggacgccctg 1020
ctgcaactta cctccgggat ggtccagacc cacgtcacca ccccaggctc cataccgacg 1080
atctgcgacc tggcgcgcac gtttgcccgg gagatggggg aggctaacta a 1131

| | |
|---|---|
| SEQ ID NO: 74 | HSVTK-amino acid |

MASYPCHQHA SAFDQAARSR GHSNRRTALR PRRQQEATEV RLEQKMPTLL RVYIDGPHGM 60
GKTTTTQLLV ALGSRDDIVY VPEPMTYWQV LGASETIANI YTTQHRLDQG EISAGDAAVV 120
MTSAQITMGM PYAVTDAVLA PHVGGEAGSS HAPPPALTLI FDRHPIAALL CYPAARYLMG 180
SMTPQAVLAF VALIPPTLPG TNIVLGALPE DRHIDRLAKR QRPGERLDLA MLAAIRRVYG 240
LLANTVRYLQ GGGSWWEDWG QLSGTAVPPQ GAEPQSNAGP RPHIGDTLFT LFRAPELLAP 300
NGDLYNVFAW ALDVLAKRLR PMHVFILDYD QSPAGCRDAL LQLTSGMVQT HVTTPGSIPT 360
ICDLARTFAR EMGEAN 376
F. dnTGFRII-CCR4(KW_L2H)-HSVTK (top) depletion system

| | |
|---|---|
| SEQ ID NO: 75 | dnTGFRII-CCR4(KW_L2H)-HSVTK-nucleotide |

atgggtcggg ggctgctcag gggcctgtgg ccgctgcaca tcgtcctgtg gacgcgtatc 60
gccagcacga tcccaccgca cgttcagaag tcggatgtgg aaatggaggc ccagaaagat 120
gaaatcatct gccccagctg taataggact gcccatccac tgagacatat taataacgac 180
atgatagtca ctgacaacaa cggtgcagtc aagtttccac aactgtgtaa attttgtgat 240
gtgagatttt ccacctgtga caaccagaaa tcctgcatga gcaactgcag catcacctcc 300
atctgtgaga agccacagga agtctgtgtg gctgtatgga gaaagaatga cgagaacata 360
acactagaga cagtttgcca tgaccccaag ctcccctacc atgactttat tctggaagat 420
gctgcttctc caaagtgcat tatgaaggaa aaaaaaaagc ctggtgagac tttcttcatg 480
tgttcctgta gctctgatga gtgcaatgac aacatcatct tctcagaaga atataacacc 540
agcaatcctg acttgttgct agtcatattt caagtgacag gcatcagcct cctgccacca 600
ctgggagttg ccatatctgt catcatcatc ttctactgct accgcgttaa ccggcagcag 660
aagctgggca gcggcgaggg cagaggcagc ctgctgacct gcggcgacgt ggaggagaac 720
cccggaccta tggccctgcc tgtgacagcc ctgctgctgc ctctggctct gctgctgcat 780
gccgctagac ccggatccga cgtgctgatg acacagagcc ctctgagcct gcctgtgaca 840
cctggcgaac ctgccagcat cagctgcaga tccagccgga acatcgtgca catcaacggc 900
gacacctacc tggaatggta tctgcagaag cccggccagt ctcctcagct gctgatctac 960
aaggtgtcca accggttcag cggcgtgccc gatagatttt ctggcagcgg ctctggcacc 1020
gacttcaccc tgaagatctc cagagtggaa gccgaggacg tgggcgtgta ctactgcttc 1080
caaggctctc tgctgccctg gacatttggc cagggcacca aggtggaaat caagggagga 1140
ggtggatcag gtggaggtgg aagcggggga ggaggttccg gcggcggagg atcagaagtt 1200
cagctggttg agtctggcgg cgacctggtt cagcctggca gatctctgag actgagctgt 1260
gccgccagcg gcttcatctt cagcaactac ggcatgagct gggtccgaca ggcccctgga 1320
aaaggactgg aatgggtcgc cacaatcagc agcgccagca cctacagcta ctaccccgat 1380
agcgtgaagg gcagattcac catcagccgg gacaacgcca agaacagcct gtacctgcag 1440
atgaactccc tgcgcgtgga agatacagcc ctgtactatt gcggcagaca cagcgacggc 1500
aacttcgcct ttggctattg gggccaggga accctggtca ccgtttctag ttccggaacc 1560
acgacgccag cgccgcgacc accaacaccg gcgcccacca tcgcgtcgca gcccctgtcc 1620
ctgcgcccag aggcgtgccg gccagcggcg gggggcgcag tgcacacgag ggggctggac 1680
ttcgcctgtg atatctacat ctgggcgccc ttggccggga cttgtggggt ccttctcctg 1740
tcactggtta tcacccttta ctgcaaacgg ggcagaaaga aactcctgta tatattcaaa 1800
caaccattta tgagaccagt acaaactact caagaggaag atggctgtag ctgccgattt 1860
ccagaagaag aagaaggagg atgtgaactg agagtgaagt tcagcaggag cgcagacgcc 1920
cccgcgtaca agcagggcca gaaccagctc tataacgagc tcaatctagg acgaagagag 1980
gagtacgatg ttttggacaa gagacgtggc cgggaccctg agatgggggg aaagccgaga 2040
aggaagaacc ctcaggaagg cctgtacaat gaactgcaga aagataagat ggcggaggcc 2100
tacagtgaga ttgggatgaa aggcgagcgc cggaggggca aggggcacga tggcctttac 2160
cagggtctca gtacagccac caaggacacc tacgacgccc ttcacatgca ggccctgccc 2220
cctcgcggaa gcggagctac taacttcagc ctgctgaagc aggctggaga cgtggaggag 2280
aaccctggac ctatggcttc gtacccctgc catcaacacg cgtctgcgtt cgaccaggct 2340
gcgcgttctc gcggccatag caaccgacgt acggcgttgc gccctcgccg gcagcaagaa 2400
gccacggaag tccgcctgga gcagaaaatg cccacgctac tgcgggttta tatagacggt 2460
cctcacggga tggggaaaac caccaccacg caactgctgg tggccctggg ttcgcgcgac 2520
gatatcgtct acgtacccga gccgatgact tactggcagg tgctgggggc ttccgagaca 2580
atcgcgaaca tctacaccac acaacaccgc ctcgaccagg gcgagatatc ggccggggac 2640
gcggcggtgg taatgacaag cgcccagata acaatgggca tgccttatgc cgtgaccgac 2700
gccgttctgg ctcctcatgt cgggggggag gctgggagtt cacatgcccc gcccccggcc 2760
ctcaccctca tcttcgaccg ccatcccatc gccgccctcc tgtgctaccc ggccgcgcga 2820
taccttatgg gcagcatgac cccccaggcc gtgctggcgt tcgtggccct catcccgccg 2880
accttgcccg gcacaaacat cgtgttgggg gcccttccgg aggacagaca catcgaccgc 2940
ctggccaaac gccagcgccc cggcgagcgg cttgacctgg ctatgctggc cgcgattcgc 3000
cgcgtttacg ggctgcttgc caatacggtg cggtatctgc agggcggcgg gtcgtggtgg 3060
gaggattggg gacagctttc ggggacggcc gtgccgcccc agggtgccga gccccagagc 3120
aacgcgggcc cacgacccca tatcggggac acgttattta ccctgtttcg ggcccccgag 3180
ttgctggccc ccaacggcga cctgtataac gtgtttgcct gggccttgga cgtcttggcc 3240
aaacgcctcc gtcccatgca cgtctttatc ctggattacg accaatcgcc cgccggctgc 3300
cgggacgccc tgctgcaact tacctccggg atggtccaga cccacgtcac caccccaggc 3360
tccataccga cgatctgcga cctggcgcgc acgtttgccc gggagatggg ggaggctaac 3420
taa 3423

| | |
|---|---|
| SEQ ID NO: 76 | dnTGFRII-CCR4(KW_L2H)-HSVTK-amino acid |

MGRGLLRGLW PLHIVLWTRI ASTIPPHVQK SDVEMEAQKD EIICPSCNRT AHPLRHINND 60
MIVTDNNGAV KFPQLCKFCD VRESTCDNQK SCMSNCSITS ICEKPQEVCV AVWRKNDENI 120
TLETVCHDPK LPYHDFILED AASPKCIMKE KKKPGETFFM CSCSSDECND NIIFSEEYNT 180
SNPDLLLVIF QVTGISLLPP LGVAISVIII FYCYRVNRQQ KLGSGEGRGS LLTCGDVEEN 240
PGPMALPVTA LLLPLALLLH AARPGSDVLM TQSPLSLPVT PGEPASISCR SSRNIVHING 300
DTYLEWYLQK PGQSPQLLIY KVSNRFSGVP DRFSGSGSGT DFTLKISRVE AEDVGVYYCF 360
QGSLLPWTFG QGTKVEIKGG GGSGGGGSGG GGSGGGGSEV QLVESGGDLV QPGRSLRLSC 420
AASGFIFSNY GMSWVRQAPG KGLEWVATIS SASTYSYYPD SVKGRFTISR DNAKNSLYLQ 480
MNSLRVEDTA LYYCGRHSDG NFAFGYWGQG TLVTVSSSGT TTPAPRPPTP APTIASQPLS 540
LRPEACRPAA GGAVHTRGLD FACDIYIWAP LAGTCGVLLL SLVITLYCKR GRKKLLYIFK 600
QPFMRPVQTT QEEDGCSCRF PEEEEGGCEL RVKFSRSADA PAYKQGQNQL YNELNLGRRE 660
EYDVLDKRRG RDPEMGGKPR RKNPQEGLYN ELQKDKMAEA YSEIGMKGER RRGKGHDGLY 720
QGLSTATKDT YDALHMQALP PRGSGATNFS LLKQAGDVEE NPGPMASYPC HQHASAFDQA 780
ARSRGHSNRR TALRPRRQQE ATEVRLEQKM PTLLRVYIDG PHGMGKTTTT QLLVALGSRD 840
DIVYVPEPMT YWQVLGASET IANIYTTQHR LDQGEISAGD AAVVMTSAQI TMGMPYAVTD 900
AVLAPHVGGE AGSSHAPPPA LTLIFDRHPI AALLCYPAAR YLMGSMTPQA VLAFVALIPP 960
TLPGTNIVLG ALPEDRHIDR LAKRQRPGER LDLAMLAAIR RVYGLLANTV RYLQGGGSWW 1020
EDWGQLSGTA VPPQGAEPQS NAGPRPHIGD TLFTLFRAPE LLAPNGDLYN VFAWALDVLA 1080
KRLRPMHVFI LDYDQSPAGC RDALLQLTSG MVQTHVTTPG SIPTICDLAR TFAREMGEAN 1140

| | |
|---|---|
| SEQ ID NO: 77 | dnTGNbRII-nucleotide |

atgggtcggg ggctgctcag gggcctgtgg ccgctgcaca tcgtcctgtg gacgcgtatc 60
gccagcacga tcccaccgca cgttcagaag tcggatgtgg aaatggaggc ccagaaagat 120
gaaatcatct gccccagctg taataggact gcccatccac tgagacatat taataacgac 180
atgatagtca ctgacaacaa cggtgcagtc aagtttccac aactgtgtaa attttgtgat 240
gtgagatttt ccacctgtga caaccagaaa tcctgcatga gcaactgcag catcacctcc 300
atctgtgaga agccacagga agtctgtgtg gctgtatgga gaaagaatga cgagaacata 360
acactagaga cagtttgcca tgaccccaag ctcccctacc atgactttat tctggaagat 420
gctgcttctc caaagtgcat tatgaaggaa aaaaaaaagc ctggtgagac tttcttcatg 480
tgttcctgta gctctgatga gtgcaatgac aacatcatct tctcagaaga atataacacc 540
agcaatcctg acttgttgct agtcatattt caagtgacag gcatcagcct cctgccacca 600
ctgggagttg ccatatctgt catcatcatc ttctactgct accgcgttaa ccggcagcag 660
aagctg 666

| | |
|---|---|
| SEQ ID NO: 78 | dnTGNbRII-amino acid |

MGRGLLRGLW PLHIVLWTRI ASTIPPHVQK SDVEMEAQKD EIICPSCNRT AHPLRHINND 60
MIVTDNNGAV KFPQLCKFCD VRESTCDNQK SCMSNCSITS ICEKPQEVCV AVWRKNDENI 120
TLETVCHDPK LPYHDFILED AASPKCIMKE KKKPGETFFM CSCSSDECND NIIFSEEYNT 180
SNPDLLLVIF QVTGISLIPP LGVAISVIII FYCYRVNRQQ KL 222

| | |
|---|---|
| SEQ ID NO: 79 | T2A-nucleotide |

ggcagcggcg agggcagagg cagcctgctg acctgcggcg acgtggagga gaaccccgga 60
cct 63

| | |
|---|---|
| SEQ ID NO: 80 | T2A-amino acid |

GSGEGRGSLL TCGDVEENPG P 21

| | |
|---|---|
| SEQ ID NO: 81 | CCR4(KW_L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgc 1497

| | |
|---|---|
| SEQ ID NO: 82 | CCR4(KW_L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPR 499

| | |
|---|---|
| SEQ ID NO: 83 | P2A-nucleotide |

ggaagcggag ctactaactt cagcctgctg aagcaggctg gagacgtgga ggagaaccct 60
ggacct 66

| | |
|---|---|
| SEQ ID NO: 84 | P2A-amino acid |

GSGATNFSLL KQAGDVEENP GP 22

| | |
|---|---|
| SEQ ID NO: 85 | HSVTK-nucleotide |

atggcttcgt acccctgcca tcaacacgcg tctgcgttcg accaggctgc gcgttctcgc 60
ggccatagca accgacgtac ggcgttgcgc cctcgccggc agcaagaagc cacggaagtc 120
cgcctggagc agaaaatgcc cacgctactg cgggtttata tagacggtcc tcacgggatg 180
gggaaaacca ccaccacgca actgctggtg gccctgggtt cgcgcgacga tatcgtctac 240
gtacccgagc cgatgactta ctggcaggtg ctgggggctt ccgagacaat cgcgaacatc 300
tacaccacac aacaccgcct cgaccagggc gagatatcgg ccggggacgc ggcggtggta 360
atgacaagcg cccagataac aatgggcatg ccttatgccg tgaccgacgc cgttctggct 420
cctcatgtcg ggggggaggc tgggagttca catgccccgc ccccggccct caccctcatc 480
ttcgaccgcc atcccatcgc cgccctcctg tgctacccgg ccgcgcgata ccttatgggc 540
agcatgaccc cccaggccgt gctggcgttc gtggccctca tcccgccgac cttgcccggc 600
acaaacatcg tgttgggggc ccttccggag gacagacaca tcgaccgcct ggccaaacgc 660
cagcgccccg gcgagcggct tgacctggct atgctggccg cgattcgccg cgtttacggg 720
ctgcttgcca atacggtgcg gtatctgcag ggcggcgggt cgtggtggga ggattgggga 780
cagctttcgg ggacggccgt gccgccccag ggtgccgagc cccagagcaa cgcgggccca 840
cgaccccata tcggggacac gttatttacc ctgtttcggg cccccgagtt gctggccccc 900
aacggcgacc tgtataacgt gtttgcctgg gccttggacg tcttggccaa acgcctccgt 960
cccatgcacg tctttatcct ggattacgac caatcgcccg ccggctgccg ggacgccctg 1020
ctgcaactta cctccgggat ggtccagacc cacgtcacca ccccaggctc cataccgacg 1080
atctgcgacc tggcgcgcac gtttgcccgg gagatggggg aggctaacta a 1131

| | |
|---|---|
| SEQ ID NO: 86 | HSVTK-amino acid |

MASYPCHQHA SAFDQAARSR GHSNRRTALR PRRQQEATEV RLEQKMPTLL RVYIDGPHGM 60
GKTTTTQLLV ALGSRDDIVY VPEPMTYWQV LGASETIANI YTTQHRLDQG EISAGDAAVV 120
MTSAQITMGM PYAVTDAVLA PHVGGEAGSS HAPPPALTLI FDRHPIAALL CYPAARYLMG 180
SMTPQAVLAF VALIPPTLPG TNIVLGALPE DRHIDRLAKR QRPGERLDLA MLAAIRRVYG 240
LLANTVRYLQ GGGSWWEDWG QLSGTAVPPQ GAEPQSNAGP RPHIGDTLFT LFRAPELLAP 300
NGDLYNVFAW ALDVLAKRLR PMHVFILDYD QSPAGCRDAL LQLTSGMVQT HVTTPGSIPT 360
ICDLARTFAR EMGEAN 376
G. CCR4(KW_L2H)-dnTGFbRII-HSVTK (mid) depletion system

| | |
|---|---|
| SEQ ID NO: 87 | CCR4(KW_L2H)-dnTGFbRII-HSVTK-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgcgga 1500
agcggagcta ctaacttcag cctgctgaag caggctggag acgtggagga gaaccctgga 1560
cctatgggtc gggggctgct caggggcctg tggccgctgc acatcgtcct gtggacgcgt 1620
atcgccagca cgatcccacc gcacgttcag aagtcggatg tggaaatgga ggcccagaaa 1680
gatgaaatca tctgccccag ctgtaatagg actgcccatc cactgagaca tattaataac 1740
gacatgatag tcactgacaa caacggtgca gtcaagtttc cacaactgtg taaattttgt 1800
gatgtgagat tttccacctg tgacaaccag aaatcctgca tgagcaactg cagcatcacc 1860
tccatctgtg agaagccaca ggaagtctgt gtggctgtat ggagaaagaa tgacgagaac 1920
ataacactag agacagtttg ccatgacccc aagctcccct accatgactt tattctggaa 1980
gatgctgctt ctccaaagtg cattatgaag gaaaaaaaaa agcctggtga gactttcttc 2040
atgtgttcct gtagctctga tgagtgcaat gacaacatca tcttctcaga agaatataac 2100
accagcaatc ctgacttgtt gctagtcata tttcaagtga caggcatcag cctcctgcca 2160
ccactgggag ttgccatatc tgtcatcatc atcttctact gctaccgcgt taaccggcag 2220
cagaagctgg gcagcggcga gggcagaggc agcctgctga cctgcggcga cgtggaggag 2280
aaccccggac ctatggcttc gtacccctgc catcaacacg cgtctgcgtt cgaccaggct 2340
gcgcgttctc gcggccatag caaccgacgt acggcgttgc gccctcgccg gcagcaagaa 2400
gccacggaag tccgcctgga gcagaaaatg cccacgctac tgcgggttta tatagacggt 2460
cctcacggga tggggaaaac caccaccacg caactgctgg tggccctggg ttcgcgcgac 2520
gatatcgtct acgtacccga gccgatgact tactggcagg tgctgggggc ttccgagaca 2580
atcgcgaaca tctacaccac acaacaccgc ctcgaccagg gcgagatatc ggccggggac 2640
gcggcggtgg taatgacaag cgcccagata acaatgggca tgccttatgc cgtgaccgac 2700
gccgttctgg ctcctcatgt cgggggggag gctgggagtt cacatgcccc gcccccggcc 2760
ctcaccctca tcttcgaccg ccatcccatc gccgccctcc tgtgctaccc ggccgcgcga 2820
taccttatgg gcagcatgac cccccaggcc gtgctggcgt tcgtggccct catcccgccg 2880
accttgcccg gcacaaacat cgtgttgggg gcccttccgg aggacagaca catcgaccgc 2940
ctggccaaac gccagcgccc cggcgagcgg cttgacctgg ctatgctggc cgcgattcgc 3000
cgcgtttacg ggctgcttgc caatacggtg cggtatctgc agggcggcgg gtcgtggtgg 3060
gaggattggg gacagctttc ggggacggcc gtgccgcccc agggtgccga gccccagagc 3120
aacgcgggcc cacgacccca tatcggggac acgttattta ccctgtttcg ggcccccgag 3180
ttgctggccc ccaacggcga cctgtataac gtgtttgcct gggccttgga cgtcttggcc 3240
aaacgcctcc gtcccatgca cgtctttatc ctggattacg accaatcgcc cgccggctgc 3300
cgggacgccc tgctgcaact tacctccggg atggtccaga cccacgtcac caccccaggc 3360
tccataccga cgatctgcga cctggcgcgc acgtttgccc gggagatggg ggaggctaac 3420
taa 3423

| | |
|---|---|
| SEQ ID NO: 88 | CCR4(KW L2H)-dnTGFbRII-HSVTK-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDET LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRG SGATNFSLLK QAGDVEENPG PMGRGLLRGL WPLHIVLWTR 540
IASTIPPHVQ KSDVEMEAQK DEIICPSCNR TAHPLRHINN DMIVTDNNGA VKFPQLCKFC 600
DVRFSTCDNQ KSCMSNCSIT SICEKPQEVC VAVWRKNDEN ITLETVCHDP KLPYHDFILE 660
DAASPKCIMK EKKKPGETFF MCSCSSDECN DNIIFSEEYN TSNPDLLLVI FQVTGISLLP 720
PLGVAISVII IFYCYRVNRQ QKLGSGEGRG SLLTCGDVEE NPGPMASYPC HQHASAFDQA 780
ARSRGHSNRR TALRPRRQQE ATEVRLEQKM PTLLRVYIDG PHGMGKTTTT QLLVALGSRD 840
DIVYVPEPMT YWQVLGASET IANIYTTQHR LDQGEISAGD AAVVMTSAQI TMGMPYAVTD 900
AVLAPHVGGE AGSSHAPPPA LTLIFDRHPI AALLCYPAAR YLMGSMTPQA VLAFVALIPP 960
TLPGTNIVLG ALPEDRHIDR LAKRQRPGER LDLAMLAAIR RVYGLLANTV RYLQGGGSWW 1020
EDWGQLSGTA VPPQGAEPQS NAGPRPHIGD TLFTLFRAPE LLAPNGDLYN VFAWALDVLA 1080
KRLRPMHVFI LDYDQSPAGC RDALLQLTSG MVQTHVTTPG SIPTICDLAR TFAREMGEAN 1140

| | |
|---|---|
| SEQ ID NO: 89 | CCR4(KW L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgc 1497

| | |
|---|---|
| SEQ ID NO: 90 | CCR4(KW_L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPR 499

| | |
|---|---|
| SEQ ID NO: 91 | P2A-nucleotide |

ggaagcggag ctactaactt cagcctgctg aagcaggctg gagacgtgga ggagaaccct 60
ggacct 66

| | |
|---|---|
| SEQ ID NO: 92 | P2A-amino acid |

GSGATNFSLL KQAGDVEENP GP 22

| | |
|---|---|
| SEQ ID NO: 93 | dnTGFbRII-nucleotide |

atgggtcggg ggctgctcag gggcctgtgg ccgctgcaca tcgtcctgtg gacgcgtatc 60
gccagcacga tcccaccgca cgttcagaag tcggatgtgg aaatggaggc ccagaaagat 120
gaaatcatct gccccagctg taataggact gcccatccac tgagacatat taataacgac 180
atgatagtca ctgacaacaa cggtgcagtc aagtttccac aactgtgtaa attttgtgat 240
gtgagatttt ccacctgtga caaccagaaa tcctgcatga gcaactgcag catcacctcc 300
atctgtgaga agccacagga agtctgtgtg gctgtatgga gaaagaatga cgagaacata 360
acactagaga cagtttgcca tgaccccaag ctcccctacc atgactttat tctggaagat 420
gctgcttctc caaagtgcat tatgaaggaa aaaaaaaagc ctggtgagac tttcttcatg 480
tgttcctgta gctctgatga gtgcaatgac aacatcatct tctcagaaga atataacacc 540
agcaatcctg acttgttgct agtcatattt caagtgacag gcatcagcct cctgccacca 600
ctgggagttg ccatatctgt catcatcatc ttctactgct accgcgttaa ccggcagcag 660
aagctg 666

| | |
|---|---|
| SEQ ID NO: 94 | dnTGFbRII-amino acid |

MGRGLLRGLW PLHIVLWTRI ASTIPPHVQK SDVEMEAQKD EIICPSCNRT AHPLRHINND 60
MIVTDNNGAV KFPQLCKFCD VRFSTCDNQK SCMSNCSITS ICEKPQEVCV AVWRKNDENI 120
TLETVCHDPK LPYHDFILED AASPKCIMKE KKKPGETFFM CSCSSDECND NIIFSEEYNT 180
SNPDLLLVIF QVTGISLLPP LGVAISVIII FYCYRVNRQQ KL 222

| | |
|---|---|
| SEQ ID NO: 95 | T2A-nucleotide |

ggcagcggcg agggcagagg cagcctgctg acctgcggcg acgtggagga gaaccccgga 60
cct 63

| | |
|---|---|
| SEQ ID NO: 96 | T2A-amino acid |

GSGEGRGSLL TCGDVEENPG P 21

| | |
|---|---|
| SEQ ID NO: 97 | HSVTK-nucleotide |

atggcttcgt acccctgcca tcaacacgcg tctgcgttcg accaggctgc gcgttctcgc 60
ggccatagca accgacgtac ggcgttgcgc cctcgccggc agcaagaagc cacggaagtc 120
cgcctggagc agaaaatgcc cacgctactg cgggtttata tagacggtcc tcacgggatg 180
gggaaaacca ccaccacgca actgctggtg gccctgggtt cgcgcgacga tatcgtctac 240
gtacccgagc cgatgactta ctggcaggtg ctgggggctt ccgagacaat cgcgaacatc 300
tacaccacac aacaccgcct cgaccagggc gagatatcgg ccggggacgc ggcggtggta 360
atgacaagcg cccagataac aatgggcatg ccttatgccg tgaccgacgc cgttctggct 420
cctcatgtcg ggggggaggc tgggagttca catgccccgc ccccggccct caccctcatc 480
ttcgaccgcc atcccatcgc cgccctcctg tgctacccgg ccgcgcgata ccttatgggc 540
agcatgaccc cccaggccgt gctggcgttc gtggccctca tcccgccgac cttgcccggc 600
acaaacatcg tgttgggggc ccttccggag gacagacaca tcgaccgcct ggccaaacgc 660
cagcgccccg gcgagcggct tgacctggct atgctggccg cgattcgccg cgtttacggg 720
ctgcttgcca atacggtgcg gtatctgcag ggcggcgggt cgtggtggga ggattgggga 780
cagctttcgg ggacggccgt gccgccccag ggtgccgagc cccagagcaa cgcgggccca 840
cgaccccata tcggggacac gttatttacc ctgtttcggg cccccgagtt gctggccccc 900
aacggcgacc tgtataacgt gtttgcctgg gccttggacg tcttggccaa acgcctccgt 960
cccatgcacg tctttatcct ggattacgac caatcgcccg ccggctgccg ggacgccctg 1020
ctgcaactta cctccgggat ggtccagacc cacgtcacca ccccaggctc cataccgacg 1080
atctgcgacc tggcgcgcac gtttgcccgg gagatggggg aggctaacta a 1131

| | |
|---|---|
| SEQ ID NO: 98 | HSVTK-amino acid |

MASYPCHQHA SAFDQAARSR GHSNRRTALR PRRQQEATEV RLEQKMPTLL RVYIDGPHGM 60
GKTTTTQLLV ALGSRDDIVY VPEPMTYWQV LGASETIANI YTTQHRLDQG EISAGDAAVV 120
MTSAQITMGM PYAVTDAVLA PHVGGEAGSS HAPPPALTLI FDRHPIAALL CYPAARYLMG 180
SMTPQAVLAF VALIPPTLPG TNIVLGALPE DRHIDRLAKR QRPGERLDLA MLAAIRRVYG 240
LLANTVRYLQ GGGSWWEDWG QLSGTAVPPQ GAEPQSNAGP RPHIGDTLFT LFRAPELLAP 300
NGDLYNVFAW ALDVLAKRLR PMHVFILDYD QSPAGCRDAL LQLTSGMVQT HVTTPGSIPT 360
ICDLARTFAR EMGEAN 376
H. CCR4(KW_L2H)-HSVTK-dnTGFbRII (last) depletion system

| | |
|---|---|
| SEQ ID NO: 99 | CCR4(KW_L2H)-HSVTK-dnTGFbRII-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgcgga 1500
agcggagcta ctaacttcag cctgctgaag caggctggag acgtggagga gaaccctgga 1560
cctatggctt cgtacccctg ccatcaacac gcgtctgcgt tcgaccaggc tgcgcgttct 1620
cgcggccata gcaaccgacg tacggcgttg cgccctcgcc ggcagcaaga agccacggaa 1680
gtccgcctgg agcagaaaat gcccacgcta ctgcgggttt atatagacgg tcctcacggg 1740
atggggaaaa ccaccaccac gcaactgctg gtggccctgg gttcgcgcga cgatatcgtc 1800
tacgtacccg agccgatgac ttactggcag gtgctggggg cttccgagac aatcgcgaac 1860
atctacacca cacaacaccg cctcgaccag ggcgagatat cggccgggga cgcggcggtg 1920
gtaatgacaa gcgcccagat aacaatgggc atgccttatg ccgtgaccga cgccgttctg 1980
gctcctcatg tcggggggga ggctgggagt tcacatgccc cgcccccggc cctcaccctc 2040
atcttcgacc gccatcccat cgccgccctc ctgtgctacc cggccgcgcg ataccttatg 2100
ggcagcatga ccccccaggc cgtgctggcg ttcgtggccc tcatcccgcc gaccttgccc 2160
ggcacaaaca tcgtgttggg ggcccttccg gaggacagac acatcgaccg cctggccaaa 2220
cgccagcgcc ccggcgagcg gcttgacctg gctatgctgg ccgcgattcg ccgcgtttac 2280
gggctgcttg ccaatacggt gcggtatctg cagggcggcg ggtcgtggtg ggaggattgg 2340
ggacagcttt cggggacggc cgtgccgccc cagggtgccg agccccagag caacgcgggc 2400
ccacgacccc atatcgggga cacgttattt accctgtttc gggcccccga gttgctggcc 2460
cccaacggcg acctgtataa cgtgtttgcc tgggccttgg acgtcttggc caaacgcctc 2520
cgtcccatgc acgtctttat cctggattac gaccaatcgc ccgccggctg ccgggacgcc 2580
ctgctgcaac ttacctccgg gatggtccag acccacgtca ccaccccagg ctccataccg 2640
acgatctgcg acctggcgcg cacgtttgcc cgggagatgg gggaggctaa cggcagcggc 2700
gagggcagag gcagcctgct gacctgcggc gacgtggagg agaaccccgg acctatgggt 2760
cgggggctgc tcaggggcct gtggccgctg cacatcgtcc tgtggacgcg tatcgccagc 2820
acgatcccac cgcacgttca gaagtcggat gtggaaatgg aggcccagaa agatgaaatc 2880
atctgcccca gctgtaatag gactgcccat ccactgagac atattaataa cgacatgata 2940
gtcactgaca acaacggtgc agtcaagttt ccacaactgt gtaaattttg tgatgtgaga 3000
ttttccacct gtgacaacca gaaatcctgc atgagcaact gcagcatcac ctccatctgt 3060
gagaagccac aggaagtctg tgtggctgta tggagaaaga atgacgagaa cataacacta 3120
gagacagttt gccatgaccc caagctcccc taccatgact ttattctgga agatgctgct 3180
tctccaaagt gcattatgaa ggaaaaaaaa aagcctggtg agactttctt catgtgttcc 3240
tgtagctctg atgagtgcaa tgacaacatc atcttctcag aagaatataa caccagcaat 3300
cctgacttgt tgctagtcat atttcaagtg acaggcatca gcctcctgcc accactggga 3360
gttgccatat ctgtcatcat catcttctac tgctaccgcg ttaaccggca gcagaagctg 3420
taa 3423

| | |
|---|---|
| SEQ ID NO: 100 | CCR4(KW_L2H)-HSVTK-dnTGFbRII-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPRG SGATNFSLLK QAGDVEENPG PMASYPCHQH ASAFDQAARS 540
RGHSNRRTAL RPRRQQEATE VRLEQKMPTL LRVYIDGPHG MGKTTTTQLL VALGSRDDIV 600
YVPEPMTYWQ VLGASETIAN IYTTQHRLDQ GEISAGDAAV VMTSAQITMG MPYAVTDAVL 660
APHVGGEAGS SHAPPPALTL IFDRHPIAAL LCYPAARYLM GSMTPQAVLA FVALIPPTLP 720
GTNIVLGALP EDRHIDRLAK RQRPGERLDL AMLAAIRRVY GLLANTVRYL QGGGSWWEDW 780
GQLSGTAVPP QGAEPQSNAG PRPHIGDTLF TLFRAPELLA PNGDLYNVFA WALDVLAKRL 840
RPMHVFILDY DQSPAGCRDA LLQLTSGMVQ THVTTPGSIP TICDLARTFA REMGEANGSG 900
EGRGSLLTCG DVEENPGPMG RGLLRGLWPL HIVLWTRIAS TIPPHVQKSD VEMEAQKDEI 960
ICPSCNRTAH PLRHINNDMI VTDNNGAVKF PQLCKFCDVR FSTCDNQKSC MSNCSITSIC 1020
EKPQEVCVAV WRKNDENITL ETVCHDPKLP YHDFILEDAA SPKCIMKEKK KPGETFFMCS 1080
CSSDECNDNI IFSEEYNTSN PDLLLVIFQV TGISLLPPLG VAISVIIIFY CYRVNRQQKL 1140

| | |
|---|---|
| SEQ ID NO: 101 | CCR4(KW L2H) CAR-nucleotide |

atggccctgc ctgtgacagc cctgctgctg cctctggctc tgctgctgca tgccgctaga 60
cccggatccg acgtgctgat gacacagagc cctctgagcc tgcctgtgac acctggcgaa 120
cctgccagca tcagctgcag atccagccgg aacatcgtgc acatcaacgg cgacacctac 180
ctggaatggt atctgcagaa gcccggccag tctcctcagc tgctgatcta caaggtgtcc 240
aaccggttca gcggcgtgcc cgatagattt tctggcagcg gctctggcac cgacttcacc 300
ctgaagatct ccagagtgga agccgaggac gtgggcgtgt actactgctt ccaaggctct 360
ctgctgccct ggacatttgg ccagggcacc aaggtggaaa tcaagggagg aggtggatca 420
ggtggaggtg gaagcggggg aggaggttcc ggcggcggag gatcagaagt tcagctggtt 480
gagtctggcg gcgacctggt tcagcctggc agatctctga gactgagctg tgccgccagc 540
ggcttcatct tcagcaacta cggcatgagc tgggtccgac aggcccctgg aaaaggactg 600
gaatgggtcg ccacaatcag cagcgccagc acctacagct actaccccga tagcgtgaag 660
ggcagattca ccatcagccg ggacaacgcc aagaacagcc tgtacctgca gatgaactcc 720
ctgcgcgtgg aagatacagc cctgtactat tgcggcagac acagcgacgg caacttcgcc 780
tttggctatt ggggccaggg aaccctggtc accgtttcta gttccggaac cacgacgcca 840
gcgccgcgac caccaacacc ggcgcccacc atcgcgtcgc agcccctgtc cctgcgccca 900
gaggcgtgcc ggccagcggc ggggggcgca gtgcacacga gggggctgga cttcgcctgt 960
gatatctaca tctgggcgcc cttggccggg acttgtgggg tccttctcct gtcactggtt 1020
atcacccttt actgcaaacg gggcagaaag aaactcctgt atatattcaa acaaccattt 1080
atgagaccag tacaaactac tcaagaggaa gatggctgta gctgccgatt tccagaagaa 1140
gaagaaggag gatgtgaact gagagtgaag ttcagcagga gcgcagacgc ccccgcgtac 1200
aagcagggcc agaaccagct ctataacgag ctcaatctag gacgaagaga ggagtacgat 1260
gttttggaca agagacgtgg ccgggaccct gagatggggg gaaagccgag aaggaagaac 1320
cctcaggaag gcctgtacaa tgaactgcag aaagataaga tggcggaggc ctacagtgag 1380
attgggatga aaggcgagcg ccggaggggc aaggggcacg atggccttta ccagggtctc 1440
agtacagcca ccaaggacac ctacgacgcc cttcacatgc aggccctgcc ccctcgc 1497

| | |
|---|---|
| SEQ ID NO: 102 | CCR4(KW_L2H) CAR-amino acid |

MALPVTALLL PLALLLHAAR PGSDVLMTQS PLSLPVTPGE PASISCRSSR NIVHINGDTY 60
LEWYLQKPGQ SPQLLIYKVS NRFSGVPDRF SGSGSGTDFT LKISRVEAED VGVYYCFQGS 120
LLPWTFGQGT KVEIKGGGGS GGGGSGGGGS GGGGSEVQLV ESGGDLVQPG RSLRLSCAAS 180
GFIFSNYGMS WVRQAPGKGL EWVATISSAS TYSYYPDSVK GRFTISRDNA KNSLYLQMNS 240
LRVEDTALYY CGRHSDGNFA FGYWGQGTLV TVSSSGTTTP APRPPTPAPT IASQPLSLRP 300
EACRPAAGGA VHTRGLDFAC DIYIWAPLAG TCGVLLLSLV ITLYCKRGRK KLLYIFKQPF 360
MRPVQTTQEE DGCSCRFPEE EEGGCELRVK FSRSADAPAY KQGQNQLYNE LNLGRREEYD 420
VLDKRRGRDP EMGGKPRRKN PQEGLYNELQ KDKMAEAYSE IGMKGERRRG KGHDGLYQGL 480
STATKDTYDA LHMQALPPR 499

| | |
|---|---|
| SEQ ID NO: 103 | P2A-nucleotide |

ggaagcggag ctactaactt cagcctgctg aagcaggctg gagacgtgga ggagaaccct 60
ggacct 66

| | |
|---|---|
| SEQ ID NO: 104 | P2A-amino acid |

GSGATNFSLL KQAGDVEENP GP 22

| | |
|---|---|
| SEQ ID NO: 105 | HSVTK-nucleotide |

atggcttcgt acccctgcca tcaacacgcg tctgcgttcg accaggctgc gcgttctcgc 60
ggccatagca accgacgtac ggcgttgcgc cctcgccggc agcaagaagc cacggaagtc 120
cgcctggagc agaaaatgcc cacgctactg cgggtttata tagacggtcc tcacgggatg 180
gggaaaacca ccaccacgca actgctggtg gccctgggtt cgcgcgacga tatcgtctac 240
gtacccgagc cgatgactta ctggcaggtg ctgggggctt ccgagacaat cgcgaacatc 300
tacaccacac aacaccgcct cgaccagggc gagatatcgg ccggggacgc ggcggtggta 360
atgacaagcg cccagataac aatgggcatg ccttatgccg tgaccgacgc cgttctggct 420
cctcatgtcg ggggggaggc tgggagttca catgccccgc ccccggccct caccctcatc 480
ttcgaccgcc atcccatcgc cgccctcctg tgctacccgg ccgcgcgata ccttatgggc 540
agcatgaccc cccaggccgt gctggcgttc gtggccctca tcccgccgac cttgcccggc 600
acaaacatcg tgttgggggc ccttccggag gacagacaca tcgaccgcct ggccaaacgc 660
cagcgccccg gcgagcggct tgacctggct atgctggccg cgattcgccg cgtttacggg 720
ctgcttgcca atacggtgcg gtatctgcag ggcggcgggt cgtggtggga ggattgggga 780
cagctttcgg ggacggccgt gccgccccag ggtgccgagc cccagagcaa cgcgggccca 840
cgaccccata tcggggacac gttatttacc ctgtttcggg cccccgagtt gctggccccc 900
aacggcgacc tgtataacgt gtttgcctgg gccttggacg tcttggccaa acgcctccgt 960
cccatgcacg tctttatcct ggattacgac caatcgcccg ccggctgccg ggacgccctg 1020
ctgcaactta cctccgggat ggtccagacc cacgtcacca ccccaggctc cataccgacg 1080
atctgcgacc tggcgcgcac gtttgcccgg gagatggggg aggctaac 1128

| | |
|---|---|
| SEQ ID NO: 106 | HSVTK-amino acid |

MASYPCHQHA SAFDQAARSR GHSNRRTALR PRRQQEATEV RLEQKMPTLL RVYIDGPHGM 60
GKTTTTQLLV ALGSRDDIVY VPEPMTYWQV LGASETIANI YTTQHRLDQG EISAGDAAVV 120
MTSAQITMGM PYAVTDAVLA PHVGGEAGSS HAPPPALTLI FDRHPIAALL CYPAARYLMG 180
SMTPQAVLAF VALIPPTLPG TNIVLGALPE DRHIDRLAKR QRPGERLDLA MLAAIRRVYG 240
LLANTVRYLQ GGGSWWEDWG QLSGTAVPPQ GAEPQSNAGP RPHIGDTLFT LFRAPELLAP 300
NGDLYNVFAW ALDVLAKRLR PMHVFILDYD QSPAGCRDAL LQLTSGMVQT HVTTPGSIPT 360
ICDLARTFAR EMGEAN 376

| | |
|---|---|
| SEQ ID NO: 107 | T2A-nucleotide |

ggcagcggcg agggcagagg cagcctgctg acctgcggcg acgtggagga gaaccccgga 60
cct 63

| | |
|---|---|
| SEQ ID NO: 108 | T2A-amino acid |

GSGEGRGSLL TCGDVEENPG P 21

| | |
|---|---|
| SEQ ID NO: 109 | dnTGFbRII-nucleotide |

atgggtcggg ggctgctcag gggcctgtgg ccgctgcaca tcgtcctgtg gacgcgtatc 60
gccagcacga tcccaccgca cgttcagaag tcggatgtgg aaatggaggc ccagaaagat 120
gaaatcatct gccccagctg taataggact gcccatccac tgagacatat taataacgac 180
atgatagtca ctgacaacaa cggtgcagtc aagtttccac aactgtgtaa attttgtgat 240
gtgagatttt ccacctgtga caaccagaaa tcctgcatga gcaactgcag catcacctcc 300
atctgtgaga agccacagga agtctgtgtg gctgtatgga gaaagaatga cgagaacata 360
acactagaga cagtttgcca tgaccccaag ctcccctacc atgactttat tctggaagat 420
gctgcttctc caaagtgcat tatgaaggaa aaaaaaaagc ctggtgagac tttcttcatg 480
tgttcctgta gctctgatga gtgcaatgac aacatcatct tctcagaaga atataacacc 540
agcaatcctg acttgttgct agtcatattt caagtgacag gcatcagcct cctgccacca 600
ctgggagttg ccatatctgt catcatcatc ttctactgct accgcgttaa ccggcagcag 660
aagctgtaa 669

| | |
|---|---|
| SEQ ID NO: 110 | dnTGFbRII-amino acid |

MGRGLLRGLW PLHIVLWTRI ASTIPPHVQK SDVEMEAQKD EIICPSCNRT AHPLRHINND 60
MIVTDNNGAV KFPQLCKFCD VRESTCDNQK SCMSNCSITS ICEKPQEVCV AVWRKNDENI 120
TLETVCHDPK LPYHDFILED AASPKCIMKE KKKPGETFFM CSCSSDECND NIIFSEEYNT 180
SNPDLLLVIF QVTGISLLPP LGVAISVIII FYCYRVNRQQ KL 222

### C. Nucleic Acids and Expression Vectors

The present disclosure provides a nucleic acid encoding a CAR. The nucleic acid of the present disclosure may comprises a polynucleotide sequence encoding any one of the CARs disclosed herein.

In certain aspects, the invention includes a nucleic acid comprising a polynucleotide sequence encoding a chimeric antigen receptor (CAR) comprising an anti-CCR4 antigen binding domain, a transmembrane domain, and an intracellular domain, wherein the antigen-binding domain comprises: a heavy chain variable region that comprises three heavy chain complementarity determining regions (HCDRs), wherein HCDR1 comprises the amino acid sequence set forth in SEQ ID NO: 2, HCDR2 comprises the amino acid sequence set forth in SEQ ID NO: 4, and HCDR3 comprises the amino acid sequence set forth in SEQ ID NO: 6; and a light chain variable region that comprises three light chain complementarity determining regions (LCDRs), wherein LCDR1 comprises the amino acid sequence set forth in SEQ ID NO: 8, LCDR2 comprises the amino acid sequence set forth in SEQ ID NO: 10, and LCDR3 comprises the amino acid sequence set forth in SEQ ID NO: 12.

In certain embodiments, the antigen binding domain comprises a heavy chain variable region encoded by a polynucleotide sequence at least 60%, 65%, 70%, 75%, 80%, 85%, 90%, 95%, 96%, 97%, 98%, 99%, or 100% identical to SEQ ID NO: 111 and/or a light chain variable region encoded by a polynucleotide sequence at least 60%, 65%, 70%, 75%, 80%, 85%, 90%, 95%, 96%, 97%, 98%, 99%, or 100% identical to SEQ ID NO: 113.

In certain embodiments, the antigen binding domain is a single-chain variable fragment (scFv) encoded by a polynucleotide sequence at least 60%, 65%, 70%, 75%, 80%, 85%, 90%, 95%, 96%, 97%, 98%, 99%, or 100% identical to SEQ ID NO: 15.

In certain embodiments, a nucleic acid of the present disclosure comprises a first polynucleotide sequence and a second polynucleotide sequence. The first and second polynucleotide sequence may be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. In certain embodiments, the nucleic acid comprises from 5′ to 3′ the first polynucleotide sequence, the linker, and the second polynucleotide sequence. In certain embodiments, the nucleic acid comprises from 5′ to 3′ the second polynucleotide sequence, the linker, and the first polynucleotide sequence.

In some embodiments, the linker comprises a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention.

In some embodiments, the linker comprises a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use in the present invention.

In some embodiments, a linker further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence. Various furin consensus recognition sequences (or “furin cleavage sites”) are known to those of skill in the art, including, without limitation, Arg-X1-Lys-Arg (SEQ ID NO:127) or Arg-X1-Arg-Arg (SEQ ID NO:128), X2-Arg-X1-X3-Arg (SEQ ID NO:129) and Arg-X1-X1-Arg (SEQ ID NO:130), such as an Arg-Gln-Lys-Arg (SEQ ID NO:131), where X1 is any naturally occurring amino acid, X2 is Lys or Arg, and X3 is Lys or Arg. Those of skill in the art would be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding a Furin cleavage site and F2A, a linker comprising a nucleic acid sequence encoding a Furin cleavage site and E2A, a linker comprising a nucleic acid sequence encoding a Furin cleavage site and P2A, a linker comprising a nucleic acid sequence encoding a Furin cleavage site and T2A. Those of skill in the art would be able to select the appropriate combination for use in the present invention. In such embodiments, the linker may further comprise a spacer sequence between the Furin cleavage site and the 2A peptide. In some embodiments, the linker comprises a Furin cleavage site 5′ to a 2A peptide. In some embodiments, the linker comprises a 2A peptide 5′ to a Furin cleavage site. Various spacer sequences are known in the art, including, without limitation, glycine serine (GS) spacers (also known as GS linkers) such as (GS)n, (SG)n, (GSGGS)n (SEQ ID NO:115) and (GGGS)n (SEQ ID NO:116), where n represents an integer of at least 1. Exemplary spacer sequences can comprise amino acid sequences including, without limitation, GGSG (SEQ ID NO:118), GGSGG (SEQ ID NO:119), GSGSG (SEQ ID NO:120), GSGGG (SEQ ID NO:121), GGGSG (SEQ ID NO:122), GSSSG (SEQ ID NO:123), and the like. Those of skill in the art would be able to select the appropriate spacer sequence for use in the present invention.

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid encoding an exogenous CAR is in operable linkage with a promoter. In certain embodiments, the promoter is a phosphoglycerate kinase-1 (PGK) promoter.

For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GALL promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Lad repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present invention may comprise a nucleic acid encoding for a CAR. In some embodiments, the expression vector (e.g., lentiviral vector) will comprise additional elements that will aid in the functional expression of the CAR encoded therein. In some embodiments, an expression vector comprising a nucleic acid encoding for a CAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR encoding nucleic acid sequence). Physiologic promoters (e.g., an EF-la promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15: 9053. Introducing RNA comprising a nucleotide sequence encoding a CAR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of a CAR as described herein, e.g., in a mammalian cell. In some embodiments, a nucleic acid of the present disclosure provides for amplification of the CAR-encoding nucleic acid.

### D. Modified Immune Cells

The present invention provides modified immune cells or precursors thereof (e.g., a T cell) comprising a CAR as described herein. Also provided are modified immune cells or precursor cell thereof comprising a nucleic acid encoding a CAR. Accordingly, such modified cells possess the specificity directed by the CAR that is expressed therein. For example, a modified cell of the present disclosure comprising a CAR possesses specificity for CCR4 on a target cell (e.g., a cancer cell). In one aspect, the invention provides a method of treating cancer comprising administering modified cells comprising an anti-CCR4 CAR to a subject having cancer, wherein the modified cells deplete CCR4-positive T cells and not CCR4-negative T cells in the subject, thereby treating the cancer.

Also provided are modified immune cells or precursor cell thereof comprising a nucleic acid encoding a CAR and further encoding a safety switch, wherein the safety switch induces CAR T cell depletion in a subject upon administration of a safety switch agent. The CAR may be linked to the safety switch via a 2A self-cleaving sequence as described herein, such as a P2A or T2A sequence. Accordingly, the safety switch depletion system comprises an anti-CCR4 CAR linked to a safety switch via a self-cleavable linker (i.e., a CAR-linked safety switch) and further comprising a safety switch agent. In one embodiment, the safety switch is truncated EGFR (EGFRt) and the safety switch agent is an anti-EGFR antibody such as cetuximab. In one embodiment, the safety switch is CD20 and the safety switch agent is an anti-CD20 antibody, such as rituximab. In one embodiment, the safety switch is inducible caspase 9 and the safety switch agent is a dimerizing drug such as AP1903. In one embodiment, the safety switch is Herpes simplex virus-1 thymidine kinase (HSVTK) and the safety switch agent is ganciclovir (GCV).

In certain embodiments, the CAR-linked safety switch further comprises a dominant-negative TGFb receptor type II (dnTGFbRII). In some embodiments, the CAR of the CAR-linked safety switch is linked to the dnTGFbRII, for example via a 2A self-cleaving sequence as described herein, such as a P2A or T2A sequence. In some embodiments, the safety switch of the CAR-linked safety switch is linked to the dnTGFbRII, for example via a 2A self-cleaving sequence as described herein, such as a P2A or T2A sequence. In some embodiments, the CAR and the safety switch of the CAR-linked safety switch are each linked to the dnTGFbRII, for example via a 2A self-cleaving sequence such as a P2A or T2A sequence. In some embodiments, the CAR-linked safety switch comprises an anti-CCR4 CAR, an HSVTK safety switch, and a dnTGFbRII, wherein the anti-CCR4 CAR, the HSVTK, and the dnTGFbRII may be present in any order from N-terminus to C-terminus of the CAR-linked safety switch.

In some embodiments, the CAR-linked safety switch comprises any one of the amino acid sequences set forth in SEQ ID NOs: 42, 52, 60, 68, 76, 88, and 100. Tolerable variations of the CAR sequences and the CAR-linked safety switch sequences will be known to those of skill in the art. For example, in certain embodiments the CAR comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in any one of SEQ ID NOs: 30, 54, 62, 70, 82, 90, and 102. In certain embodiments the CAR-linked safety switch comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the amino acid sequences set forth in SEQ ID NOs: 42, 52, 60, 68, 76, 88, and 100.

In one aspect, the invention includes a modified immune cell or precursor cell thereof, comprising a CAR comprising an anti-CCR4 antigen binding domain, a transmembrane domain, and an intracellular domain. In another aspect, the invention includes a modified immune cell or precursor cell thereof, comprising a nucleic acid encoding a CAR comprising an anti-CCR4 antigen binding domain, a transmembrane domain, and an intracellular domain. In some embodiments, the nucleic acid further encodes a safety switch, wherein the safety switch induces CAR T cell depletion in a subject upon administration of a safety switch agent.

In certain embodiments, the modified cell is a modified immune cell. In certain embodiments, the modified cell is an autologous cell. In certain embodiments, the modified cell is an autologous cell obtained from a human subject. In certain embodiments, the modified cell is a T cell.

In certain embodiments, the modified immune cell or precursor cell thereof is further modified to reduce or eliminate CCR4 expression relative to a CCR4 expression level in an unmodified immune cell. Knock down (k/d) or knock out (k/o) of CCR4 in the cells can be achieved prior to, simultaneously with, or after CCR4-CAR transduction. In some embodiments, CCR4 k/d or k/o can be achieved by any method known in the art for gene k/d or k/o, such as modification of cells with RNAi (shRNA or siRNA), genetic engineering (for example, via CRISPR/Cas9), or with protein-based systems (e.g. anti-CCR4 scFv with one or more C-terminal KDEL motifs). In some embodiments, the modified immune cell or precursor cell thereof further comprises one or more of the following: (a) a CCR4 null knockout allele; (b) suppressed CCR4 gene expression; and (c) a fusion protein comprising an anti-CCR4 scFv and a KDEL motif and/or a nucleic acid encoding the fusion protein. In certain embodiments, the CCR4 null knockout allele or suppressed CCR4 gene expression is obtained via a genetic engineering technique comprising a nuclease selected from the group consisting of a clustered regularly interspaced short palindromic repeats (CRISPR) associated nuclease, a transcription activator-like effector nuclease (TALEN), and a zinc-finger nuclease. In some embodiments, the modified immune cell or precursor cell thereof further comprises an inhibitory RNA molecule which suppresses CCR4 gene expression. Examples of inhibitory RNA molecules include, but are not limited to, an RNA interference (RNAi) RNA, a short hairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.

In some embodiments, the modified immune cell or precursor cell thereof is further modified with a CCR4-targeting RNA (e.g., shRNA) or with a nucleic acid encoding the shRNA. In some embodiments, the cell is further modified with a CCR4-targeting RNA (e.g., a guide RNA) and a Cas nuclease or with a nucleic acid encoding the CCR4-targeting RNA and the Cas nuclease. In some embodiments, the cell is further modified with an anti-CCR4 scFv-KDEL motif fusion protein or with a nucleic acid encoding an anti-CCR4 scFv-KDEL motif fusion protein.

In some embodiments, the modified immune cell further comprises a CCR4-targeting shRNA (sh-CCR4). Exemplary sh-CCR4 sequences are shown in Table 4, as well a control shRNA (i.e., a Cre recombinase-targeting shRNA sequence (sh-Cre). In some embodiments, the CCR4 is human CCR4 having mRNA sequence of NCBI Accession No. NM 005508.4. In some embodiments, the Cre gene sequence is the Cre gene sequence of NCBI Accession No. NC 005856.1. In some embodiments, the modified immune cell further comprises a CCR4-targeting shRNA comprising any one of SEQ ID NOs: 144-147 or a nucleic acid encoding a CCR4-targeting shRNA comprising any one of SEQ ID NOs: 144-147. In some embodiments, the modified immune cell further comprises a nucleic acid encoding a CCR4-targeting shRNA, wherein the nucleic acid comprises any one of SEQ ID NOs; 149-152.

TABLE 4
shRNA sequences

| SEQ ID NO. | shRNA | Targeted Region |
|---|---|---|
| 144 | sh-CCR4#1 | 1519-1541 |
| GAAUGAAGUUGUAGGUAAUAUcugugaagccacagaugggAUAUU | | |
| ACCUACAACUUCAUUCuuuuuu | | |
| 145 | sh-CCR4#2 | 1482-1504 |
| GCAUGAGUCAGUCUGAUGAGAcugugaagccacagaugggUCUCA | | |
| UCAGACUGACUCAUGCuuuuuu | | |
| 146 | sh-CCR4#3 | 1609-1631 |
| GCUGAUGGAGUAAAUCGCUACcugugaagccacagaugggGUAGC | | |
| GAUUUACUCCAUCAGCuuuuuu | | |
| 147 | sh-CCR4#4 | 1526-1548 |
| GUUGUAGGUAAUAUUGCAAGGcugugaagccacagaugggCCUUG | | |
| CAAUAUUACCUACAACuuuuuu | | |
| 148 | sh-Cre | 811-833 |
| GAUUUACGGCGCUAAGGAUGAcugugaagccacagaugggUCAUC | | |
| CUUAGCGCCGUAAAUCuuuuuu | | |
| SEQ ID NO. | DNA encoding shRNA | Targeted Region |
| 149 | sh-CCR4#1 | 1519-1541 |
| GAATGAAGTTGTAGGTAATATctgtgaagccacagatgggATATT | | |
| ACCTACAACTTCATTCtttttt | | |
| 150 | sh-CCR4#2 | 1482-1504 |
| GCATGAGTCAGTCTGATGAGActgtgaagccacagatgggTCTCA | | |
| TCAGACTGACTCATGCtttttt | | |
| 151 | sh-CCR4#3 | 1609-1631 |
| GCTGATGGAGTAAATCGCTACctgtgaagccacagatgggGTAGC | | |
| GATTTACTCCATCAGCtttttt | | |
| 152 | sh-CCR4#4 | 1526-1548 |
| GTTGTAGGTAATATTGCAAGGctgtgaagccacagatgggCCTTG | | |
| CAATATTACCTACAACtttttt | | |
| 153 | sh-Cre | 811-833 |

GATTTACGGCGCTAAGGATGActgtgaagccacagatgggTCATC
CTTAGCGCCGTAAATCtttttt

In certain embodiments, the modified immune cells are derived from peripheral blood mononuclear cells (PBMCs) of the patient. Accordingly, in some embodiments, the PBMCs comprise tumor cells. In some embodiments, the subject has previously been administered mogamulizumab. In some embodiments, the cancer is refractory to mogamulizumab.

### E. Methods of Treatment

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a modified T cell of the present invention. In another aspect, the invention includes a method of treating a disease or condition in a subject comprising administering to a subject in need thereof a population of modified T cells. In one aspect, the invention provides a method of treating cancer comprising administering modified cells comprising an anti-CCR4 CAR to a subject having cancer, wherein the modified cells deplete CCR4-positive T cells and not CCR4-negative T cells in the subject, thereby treating the cancer.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the subject has previously been administered mogamulizumab. In some embodiments, the cancer is refractory to mogamulizumab. In some embodiments, the administration effectively treats the subject despite the subject having become resistant to another therapy (e.g., mogamulizumab).

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include certain leukemia or lymphoid malignancies, benign and malignant tumors, and malignancies e.g., sarcomas, carcinomas, and melanomas. Exemplary cancers include but are not limited to mature T-cell malignancies such as adult T-cell leukemia/lymphoma (ATLL), cutaneous T-cell lymphomas (CTCLs), peripheral T-cell lymphoma (PTCL) and the like, as well as colorectal cancer, breast cancer, ovarian cancer, renal cancer, non-small cell lung cancer, melanoma, lymphoma, and hepatocellular cancers. The cancers may be non-solid tumors (such as hematological tumors) or solid tumors. Adult tumors/cancers and pediatric tumors/cancers are also included. In one embodiment, the cancer is a solid tumor or a hematological tumor. In certain embodiments, the cancer is a leukemia and/or a lymphoma. In certain embodiments, the cancer cells express CCR4.

The cells of the invention to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present invention.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In one aspect, the invention includes a method of treating cancer in a subject in need thereof, comprising administering to the subject any one of the modified immune or precursor cells disclosed herein. Yet another aspect of the invention includes a method of treating cancer in a subject in need thereof, comprising administering to the subject a modified immune or precursor cell generated by any one of the methods disclosed herein.

### F. Sources of Immune Cells

In certain embodiments, a source of immune cells (e.g. T cells) is obtained from a subject for ex vivo manipulation. Sources of target cells for ex vivo manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources. In certain embodiments, the modified immune cells are derived from peripheral blood mononuclear cells (PBMCs) of the patient. Accordingly, in some embodiments, the PBMCs comprise tumor cells.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CDl lb, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. n yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### G. Expansion of Immune Cells

Whether prior to or after modification of cells to express a CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### H. Pharmaceutical Compositions and Formulations

Also provided are populations of immune cells of the invention, compositions containing such cells and/or enriched for such cells, such as in which cells expressing CAR make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Generation of CCR4-CAR T cells: Eight types of anti-CCR4 scFvs were constructed from 4 types of anti-CCR4 mAbs. Each heavy chain variable region (H) and light chain variable region (L) were fused in either H to L or L to H orientations via 4-time repeated linkers (Table 2). ScFvs were subcloned into a second-generation backbone CAR containing 4-1BB and CD3ζ (supplementary figure S1A). A Control CD19-CAR containing 4-1BB and CD3 were constructed as previously described (Milone M C, et al. Mol Ther. 2009; 17(8):1453-1464.). T cells from normal donors were stimulated with anti-CD3/28 beads, transduced with CAR using lentivirus in the multiplicity of infection (MOI) of 4 and expanded without any cytokine supplementation.

List of CCR4-CARs and the original
mAbs from which they were derived

| | | | | Affin- | | |
|---|---|---|---|---|---|---|
| | | | mAb | ity | CCR4 | Sequence |
| # | CAR | scFv | clone | (Kd) | epitope | from |
| 1 | KW_ | H | KW- | ND | NT | WO201517 |
| | H2L | to | 0761 | (slow | 12 to | 9236A1 |
| | | L | | disso- | 29 | |
| | | | | cia- | | |
| | | | | tion) | | |
| 2 | KW_ | L | | | (DESIYSNY | |
| | L2H | to | | | YLYESIPKP | |
| | | H | | | C) | |
| 3 | 1-49_ | H | mAb | 77.98 | ND | WO201316 |
| | H2L | to | 1-49 | nM | (combi- | 6500A1 |
| | | L | | | nato- | |
| | | | | | rial | |
| | | | | | of NT | |
| | | | | | and ECL) | |
| 4 | 1-49_ | L | | | | |
| | L2H | to | | | | |
| | | H | | | | |
| 5 | 2-3_ | H | mAb | 1.39 | | |
| | H2L | to | 2-3 | nM | | |
| | | L | | | | |
| 6 | 2-3_ | L | | | | |
| | L2H | to | | | | |
| | | H | | | | |
| 7 | h1567_ | H | h1567 | 2.54 | | |
| | H2L | to | | nM | | |
| | | L | | | | |
| 8 | h1567_ | L | | | | |
| | L2H | to | | | | |
| | | H | | | | |

NT, N-terminus; ECL, extracellular loop; ND, not determined; Kd values are from data as antibodies but not as scFvs.

Cell Lines: T cell lymphoma derived cell lines, HH (ATCC CRL-2105), MJ (ATCC CRL-8294), and HuT78 (ATCC TIB-161) were obtained from the American Type Culture Collection (ATCC). A B-ALL derived cell line, Nalm6 was gift from Joseph A Fraietta at the University of Pennsylvania. Cell lines were transduced with lentivirus to express click beetle luciferase green and GFP (CBG-GFP). HE cells were further transduced to express truncated CD19 (HH-CBG-GFP-t19), then sorted by INFLUX cell sorter (BD Biosciences) for purification. To mimic CCR4 and CAR double positive T cell population, HH cells were transduced either with CCR4(KW_L2H)-CAR or CCR4(h1567_L2H)-CAR using the same lentivirus used for normal T cell transduction; HH-CCR4(KW_L2H) CAR and HHCCR4(h1567_L2H) CAR respectively. All cell lines were authenticated by the University of Arizona Genetics Core and tested for the presence of mycoplasma contamination (MycoAlert Mycoplasma Detection Kit, Lonza). HH and Nalm6 were maintained in culture with RPMI (Gibco, Life Technologies) supplemented with 10% fetal bovine serum (FBS) (Seradigm), and MJ and HuT78 were maintained in culture with IMDM (Gibco, Life Technologies) supplemented with 20% FBS.

Luciferase based killing assay: CART cells and 1×10⁴ target cells expressing luciferase were cocultured at the indicated effector target ratio (E:T ratio) in round bottom 96 well plates (Coster). Triton X-100 was added for the total lysis control. After 16-hour coculture, cells were washed with PBS and lysed with 100 ul luciferases cell culture lysis buffer (Promega) followed by one-cycle of freeze and thaw to obtain complete lysis. Lysates were analyzed for luciferase activity with Synergy H4 plate reader (BioTek) using Luciferase Assay System (Promega). Percent specific lysis was calculated using the formula; % Specific lysis=[(Experimental Lysis−Spontaneous Lysis)/(Maximum Lysis−Spontaneous Lysis)]×100.

51 Cr release assay: Target cells were labeled for 2 hours with 51 Cr (PerkinElmer) and washed twice with RPMI media. CAR T cells and 1×10⁴ labeled cells were cocultured at the indicated E:T ratio in 96 well round bottom plates for 4 hours. Sodium dodecyl sulfate (SDS) was added for the total cell lysis control. Supernatant from each well were transferred to the LumaPlate-96 (PerkinElmer) and the amount of radiation emitted by 51 Cr was determined as counts per minute (CPM) using a TopCount plate reader (PerkinElmer). Percent specific lysis was calculated using the formula; % specific lysis=[(experimental cpm−spontaneous cpm)/(maximum cpm−spontaneous cpm)]×100.

Collection of Patient Samples: Clinical samples (tumors) were obtained at the clinical practices of the University of Pennsylvania under an Institutional Review Board (IRB)-approved protocol. All subjects provided written informed consent according to the Declaration of Helsinki.

Mouse experiments and Bioluminescence imaging (BLI): Five to seven-week old NOD.Cg-Prkdc^{scid} Il2rg^{tm1Wjl}/SzJ (NSG) mice (Jackson Laboratories) were intravenously inoculated with HH-CBG-GFP or HH-CBG-GFP-t19 and treated either with 0.5 or 2×10⁶ CAR-positive T cells or un-transduced (UTD) T cells at day 7 post tumor inoculation. Tumor burden was followed by BLI in a Xenogen IVIS-200 Spectrum camera (Caliper Life Sciences) and data were analyzed with LivingImage software (PerkinElmer). The University of Pennsylvania Institutional Animal Care and Use Committee approved all animal experiments, and all animal procedures were performed in the animal facility at the University of Pennsylvania in accordance with Federal and Institutional Animal Care and Use Committee requirements.

Mouse peripheral blood analysis: Peripheral blood was obtained by retro-orbital bleeding or cardiac puncture and cell numbers of each T cell subset (total T cells, CD4, CD8) were quantified by FCM using TruCount tubes (BD Biosciences). Cytokines in serum was analyzed using high-sensitivity LUMINEX assay per the manufacturer's instructions (Merck Millipore).

Immunohistochemistry (IHC): IHC was performed on paraformaldehyde-fixed and paraffin-embedded samples. An antibody specific for CCR4 (clone: polyclonal, catalog #: HPA031613) was purchased from Sigma-Aldrich. Antibodies specific for CCL17 (EPR15861, ab195044) and CCL22 (polyclonal, ab9847) were purchased from abcm. Stained slides were scanned by ×20 magnification.

Flowcytometry (FCM) analysis and antibodies: Antibodies used for FCM analysis are listed in Table 3. Dead cells were gated out by staining with violet amine-reactive viability dye, LIVE-DEAD violet (Invitrogen). Intracellular staining was performed using the Foxp3/Transcription Factor Staining Kit (Affymetrix) per the manufacturer's instructions. CCR4-CAR and CD19-CAR expression were detected by biotinylated antihuman Fab antibody and anti-mouse Fab antibody (Jackson ImmunoResearch), respectively. Purified anti-CCR4 mAb (clone KW-0761) was conjugated with Alexa Fluor™ 680 using Alexa Fluor™ 680 Antibody Labeling Kit (Thermo Fisher Scientific). Data were collected either by a Fortessa LSRII or LSR II cytometer (BD Biosciences) and analyzed using FlowJo version 10 software (Tree Star).

TABLE 3
List of mAbs for FCM and blocking assays

| target | clone | fluorochrome | manufacturer | catalog# |
|---|---|---|---|---|
| CD19 | 4G7 | PE | BD | 349209 |
| CD26 | BA5b | PE-Cy ™5 | Biolegend | 302708 |
| CD3 | OKT3 | Briliant Violet 605 | Biolegend | 317322 |
| CD45 | 2D1 | PerCP-Cy ™5.5 | Thermo Fisher | 4334219 |
| | | | Scientific | |
| CD4 | OKT4 | Briliant Violet 510 | Biolegend | 317444 |
| CD7 | M-T701 | BV605 | BD | 740392 |
| CD7 | eBio 124-1D1 | PE-Cy ™7 | eBioscience | 25-0079-42 |
| CD8 | RPA-T8 | APC/Fire750 | Biolegend | 301066 |
| CCR10 | 1B5 | PerCP-Cy ™5.5 | BD | 564772 |
| CCR4(CD194) | D8SEE | PE | Thermo Fisher | 4346573 |
| | | | Scientific | |
| CCR4(CD194) | h1567(205410) | PE | R and D | FAB1567P |
| CCR4(CD194) | KW-0761 | purified | absolute antibody | Ab00726-23.0 |
| | (mogamulizumab) | | | |
| CCR5(CD195) | 2D7/CCR5 | PE-Cy ™7 | BD | 557752 |
| CCR6(CD196) | G04E3 | Briliant Violet 605 | Biolegend | 353420 |
| CCR7(CD197) | 3D12 | PE-Cy ™7 | BD | 557648 |
| CXCR3(CD183) | G025H7 | Alexa Fluor 488 | Biolegend | 353710 |
| CD107a | H4A3 | PE-Cy ™7 | Biolegend | 328618 |
| CD158k | 539304 | APC | R and D | FAB2878A |
| IFN-g | B27 | PE-CF594 | BD | 562392 |
| mouse IgG, Fab | na | biotin | Jackson | 115-065-072 |
| IgG, Fab | na | biotin | Jackson | 109-066-006 |
| IL-2 | MQ1-17H12 | PerCP-eFluor710 | Thermo Fisher | 46-7029-42 |
| | | | Scientific | |
| TNF-a | MAb11 | Briliant Violet 650 | Biolegend | 502938 |

Degranulation and intracellular cytokine production assay: T cells were stimulated either with control media, phorbol 12-myristate 13-acetate and ionomycin (PMA-Iono) (Sigma-Aldrich) or tumor cells at a 1:4 responder:stimulator (R:S) ratio in the existence of monensin (BD biosciences) and CD107a detection antibody. Cells were stained for LIVEDEAD violet, surface antigen then intracellular cytokines at 6 hours of co-culture as described in the FCM and antibodies section.

Statistics: Statistical analysis was performed with Prism 8 (GraphPad Software). Two-tailed Student's t test was used to compare the 2 groups and 1-way ANOVA with Tukey's post hoc test was used to compare 3 or more groups. Survival curves were drawn using the Kaplan-Meier method and the differences of 2 curves were compared with the log-rank test. P values <0.05 were considered significant.

### Example 1: CCR4 is a Reasonable Target for CART Cell Therapies

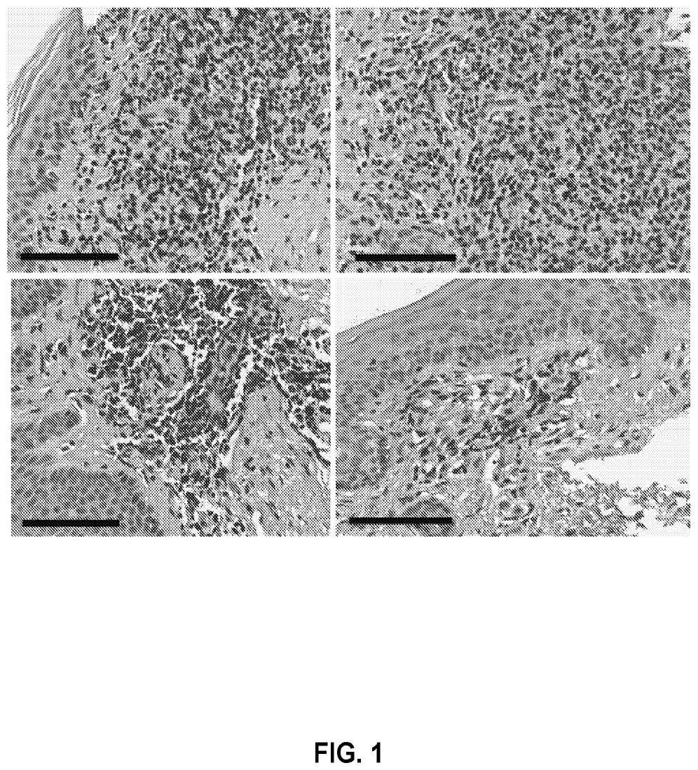

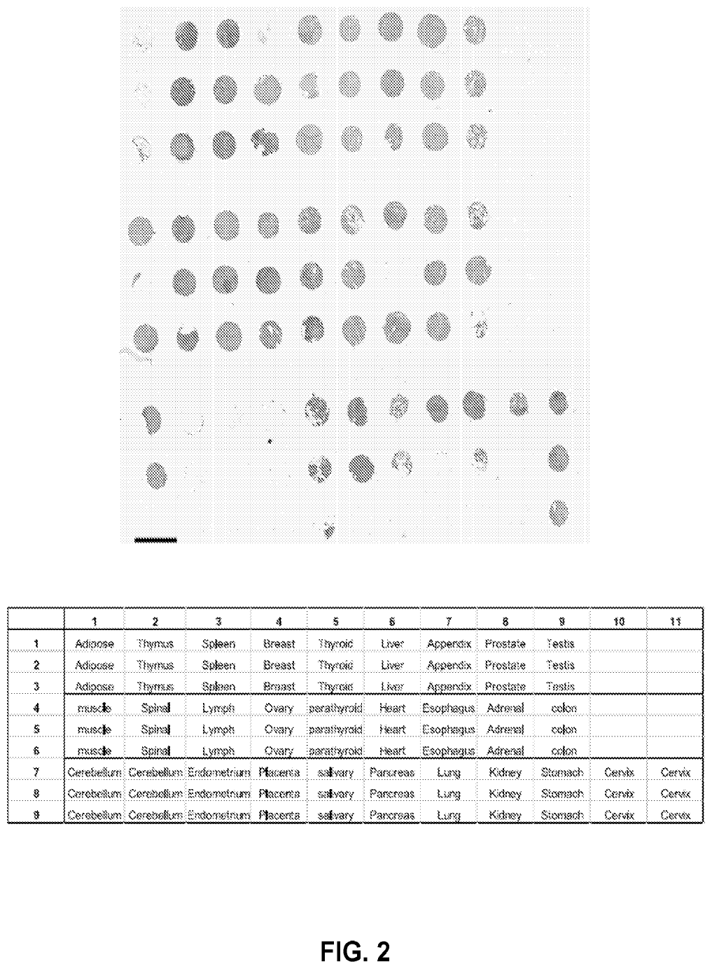

To confirm if CCR4 is a reasonable target for CART cell therapies, CCR4 expression on skin lesions from patients with CTCL and normal tissue array (TMA) derived from 27 organs was analyzed (FIG. 1 and FIG. 2). Cutaneous tumor cells of all patients were highly expressing CCR4, while normal tissues were rarely expressing CCR4. Compatible with acceptable safety profile of anti-CCR4 mAb (mogaulizumab) therapy in clinic.

### Example 2: Establishment of Eight Types of Anti-CCR4-CARs with Different scFvs

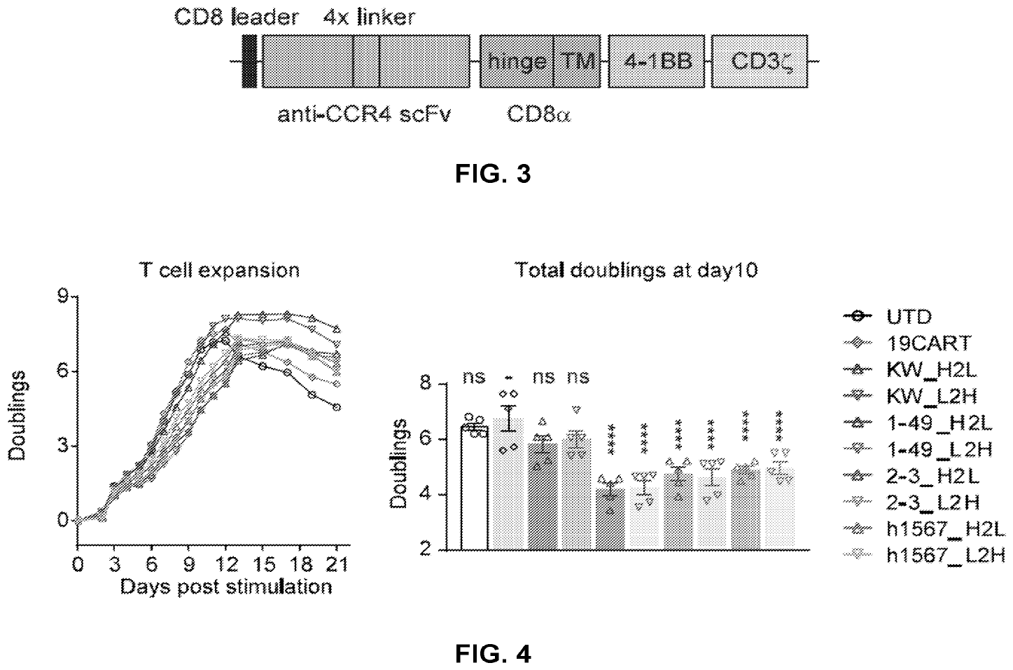

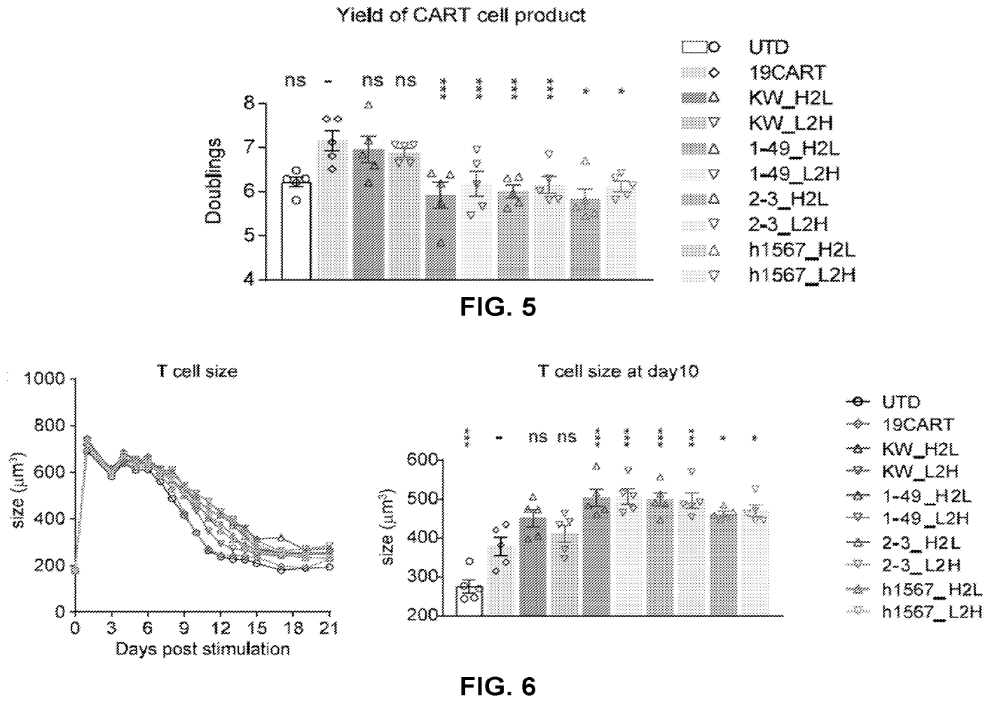

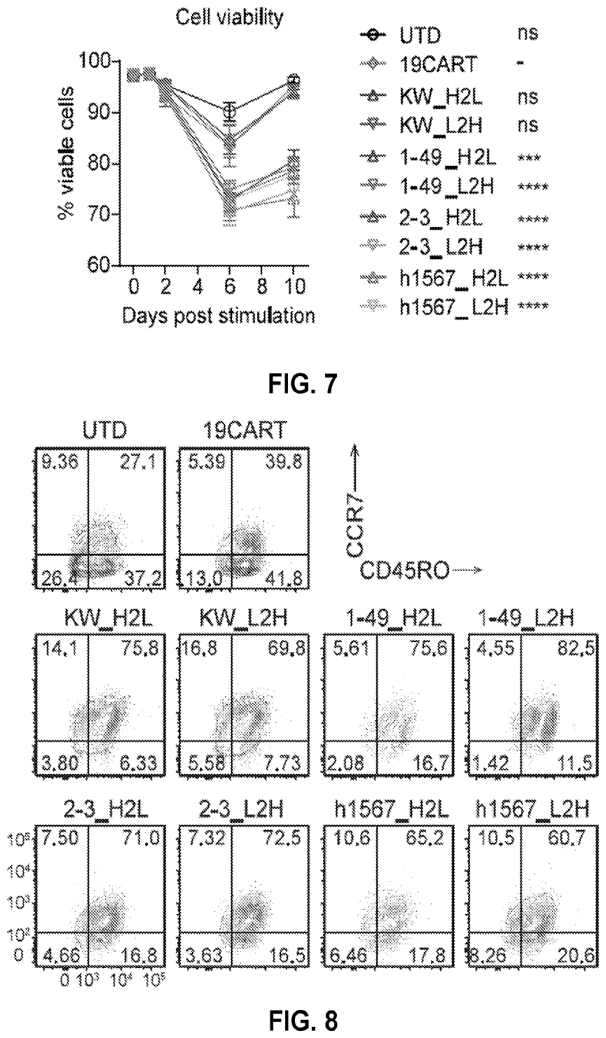

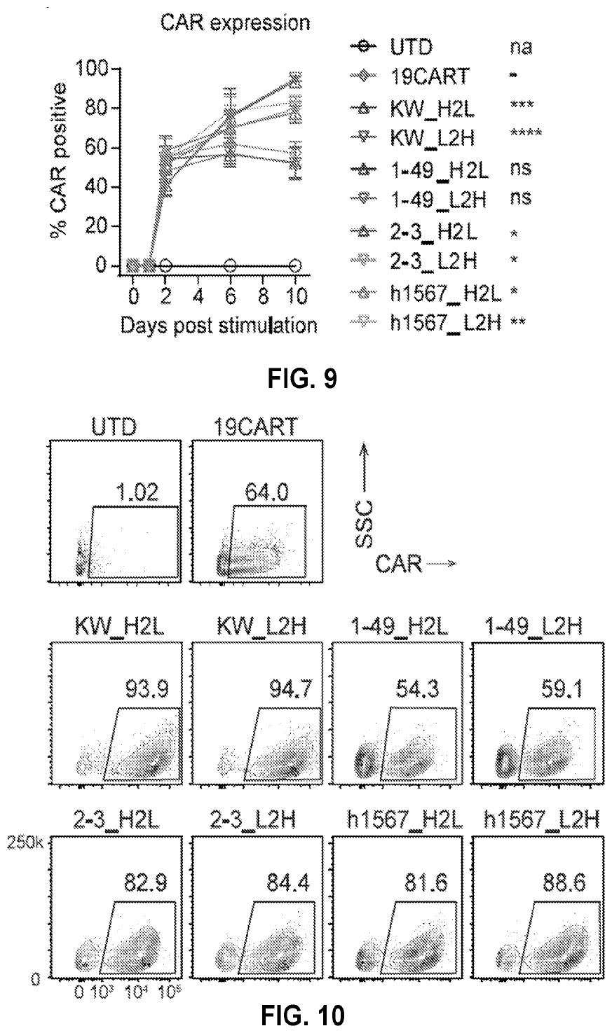

In order to generate a CAR targeting CCR4 for adoptive cell therapy of T cell lymphoma and other cancers, a panel of 8 anti-CCR4-CARs (CCR4-CARs) were engineered, each with a different spatial combination of heavy (H) and light (L) binding domains resulting from humanized anti-CCR4 monoclonal antibodies; KW-0761(mogamulizumab) (Ishii T, et al. Clin Cancer Res. 2010; 16(5):1520-1531), h1567, and its affinity-tuned derivatives (Chang D K, et al. Mol Cancer Ther. 2012; 11(11):2451-2461). The constructed scFvs were cloned into a CAR backbone plasmid with a 4-1BB costimulatory domain and CD3ζ signaling domain (Table 2 and FIG. 3). CAR transduction efficiency, T cell expansion and phenotype within the panel of CCR4-CARs were compared to the CD19 directed CAR (CD19-CAR) control. All versions of CCR4-CAR T cells expanded with anti-CD3/28 bead stimulation, however T cells expressing KW-0761 derived scFvs, CCR4(KW_H2L/L2H)-CAR T cells showed superior expansion and resulted in the highest CAR T cell product yield (FIG. 4 and FIG. 5). CCR4(KW_L2H)-CAR T cells were the smallest among 8 types of CCR4-CAR T cells (FIG. 6) suggesting less activated status. Superior T cell expansion was associated with high T cell viability (FIG. 7). All CCR4-CAR T cells kept a less differentiated, central memory phenotype compared with CD19-CAR T cells and UTD cells (FIG. 8). Interestingly, auto-selection of CAR positive cells was observed to differing degrees in six out of eight of the CCR4-CAR T cell products. In particular, CCR4(KW_H2L/L2H)-CAR T cells exhibited near 100% CAR expression at day 10 even though transduction efficacy was comparable to or lower than the other CCR4-CAR T cells at day 2 (FIG. 9 and FIG. 10).

### Example 3: CCR4(KW_H2L and KW_L2H)-CAR T Cells Induce Complete Depletion of CCR4 Positive Cells

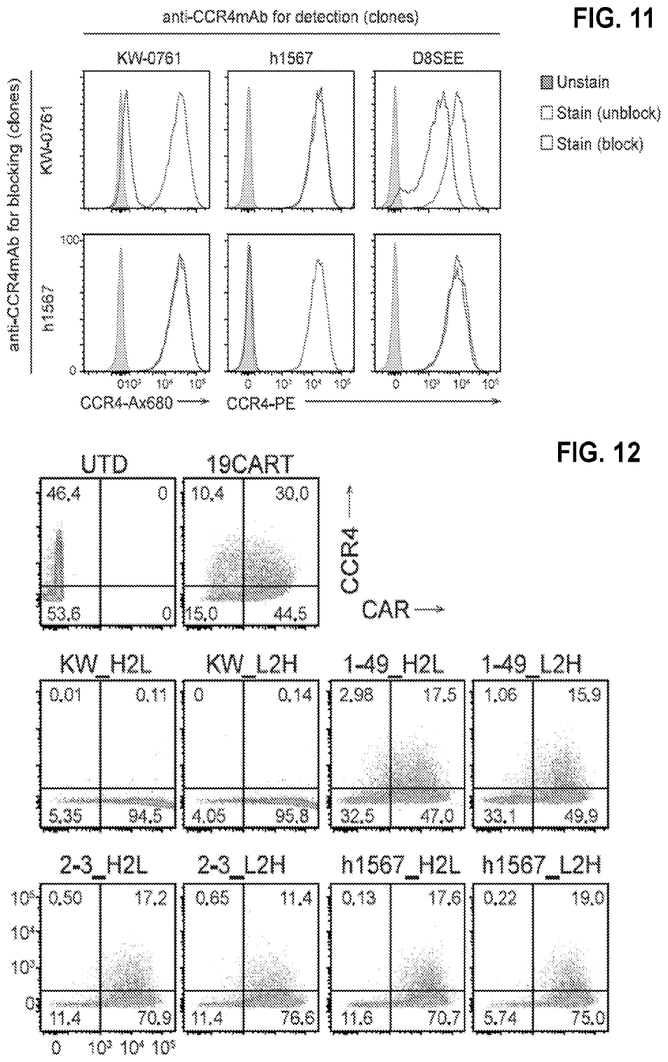

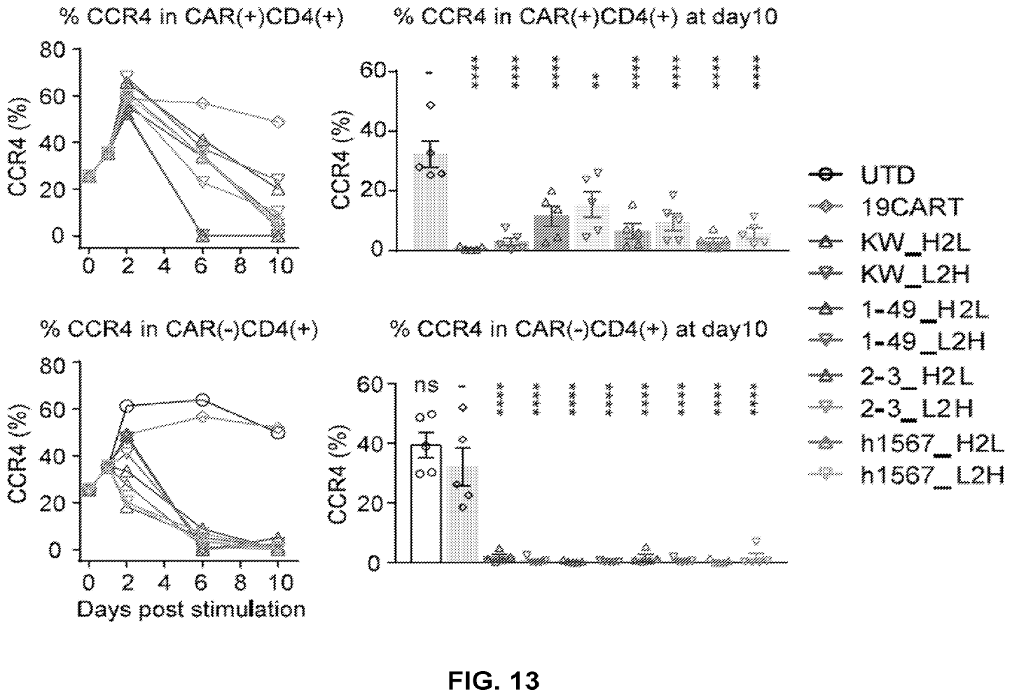

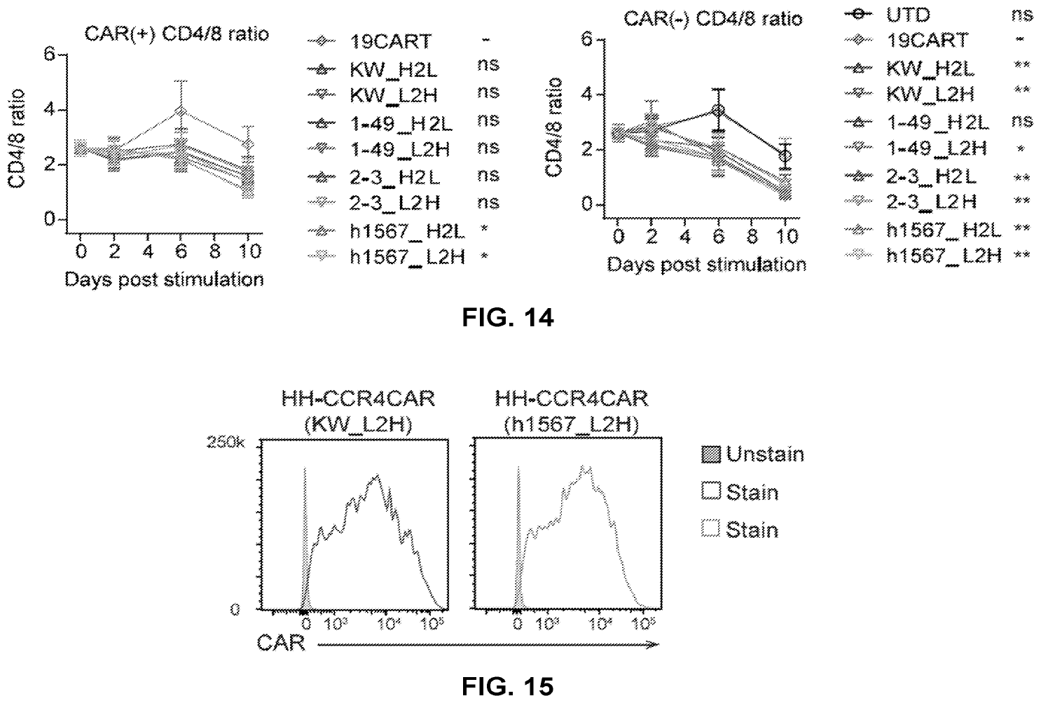

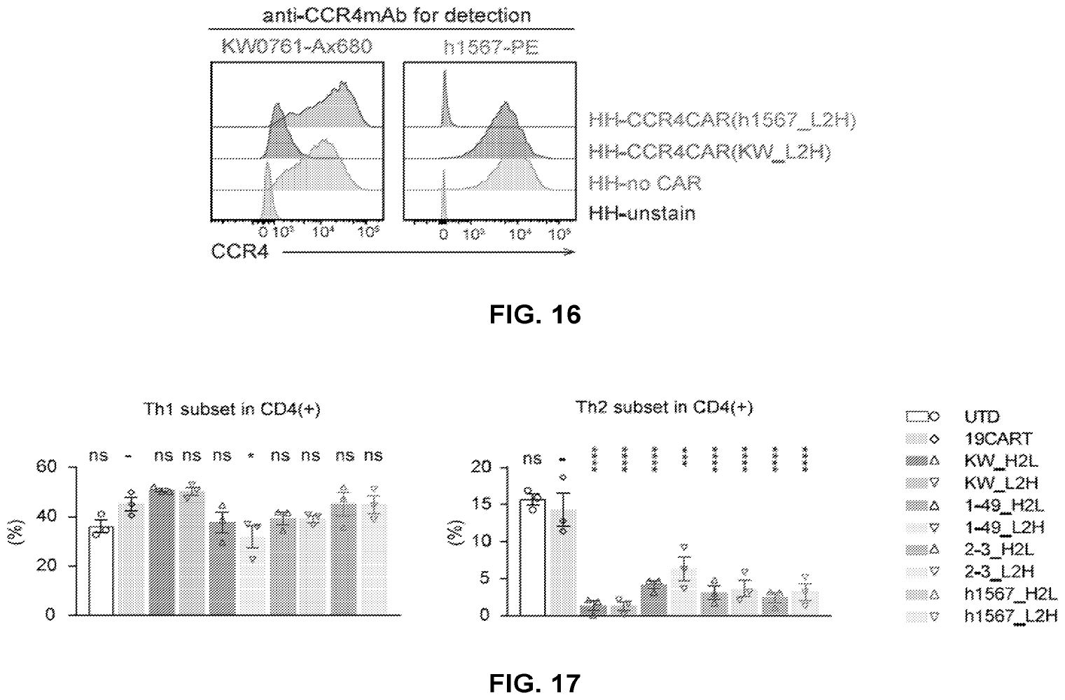

To assess whether T cell fratricide contributed to enrichment of CCR4-CAR T cell expression in CCR4-CAR T cell products, CCR4 expression levels on CAR positive and negative populations after CAR T cell expansion were measured by flow cytometry using the anti-CCR4 monoclonal antibody, D8SEE, which does not bind to the same epitope as monoclonal antibodies used for CCR4-CAR construction (FIG. 11). Rapid loss of CCR4 expression and corresponding decrease of CD4/8 ratio occurred in all CAR-negative T cell populations (FIG. 12, FIG. 13, and FIG. 14). Likewise CCR4(KW_H2L/L2H)-CAR T cells rapidly lost CCR4 expression (FIG. 12 and FIG. 13), suggesting that T cell fratricide was contributing to enrichment of CCR4-CAR positive T cells in the product. Surprisingly, CCR4(h1567, 1-49)-CAR positive cell populations were relatively resistant to fratricide; There remained high amount of CCR4 positive cells in the CAR positive population even at day 6 and day 10 (FIG. 12 and FIG. 13), suggesting that the degree of fratricide in the CAR positive population was dependent on the anti-CCR4 scFv. In order to understand why some CCR4-CAR T cells expressing surface detectable CCR4 failed to kill neighboring CCR4-expressing CCR4-CAR T cells, as opposed to other versions of CCR4-expressing CCR4-CAR T cells that were susceptible to fratricide, and to investigate the mechanisms of resistance against fratricide of CAR positive T cell population, a CCR4 positive T cell line (HH) was transduced either with CCR4(KW_L2H)-CAR or CCR4 (h1567_L2H)-CAR to mimic CCR4-CAR and CCR4 double positive T cell populations (FIG. 15). It was hypothesized that CCR4(h1567_L2H)-CAR was unable to kill CCR4-expressing CCR4(h1567_L2H)-CAR T cells because recognition of the CCR4 epitope was blocked. Thus, the binding of anti-CCR4 clone h1567 and clone KW-0761 monoclonal antibodies to HH cells expressing either CCR4(h1567_L2H)-CAR or CCR4(KW_L2H)-CARs was tested. When HH cells expressed CCR4(h1567_L2H)-CARs, anti-CCR4 clone h1567 monoclonal antibody was unable to recognize CCR4 expressed on the surface of the CAR T cell, but anti-CCR4 clone KW-0761 detected CCR4 surface expression. In HH cells expressing CCR4(KW_L2H)-CAR CCR4 detection was reduced when CCR4 expression levels were assessed using the anti-CCR4 clone KW monoclonal antibody (FIG. 17). These results suggest that CCR4-CAR blocks the CCR4 epitope specifically in cis thereby causing resistant to fratricide and indicate that the degree of binding in cis of anti-CCR4 scFv to CCR4 is scFv-dependent. This phenomenon is consistent with a previous finding that expression of CD19-CAR on CD19 positive cells can block CD19 epitope in cis, resulting in resistant against CAR mediated killing.

### Example 4: Fratricide Changes the Proportion of Helper T Cell Subsets with Decreased Th2, Th17 and Treg Related Cytokine Production

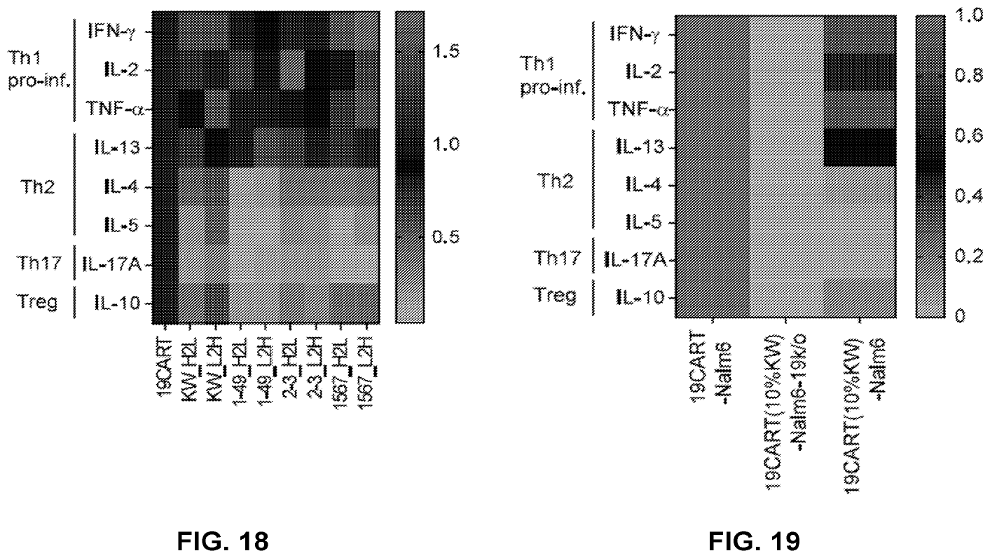

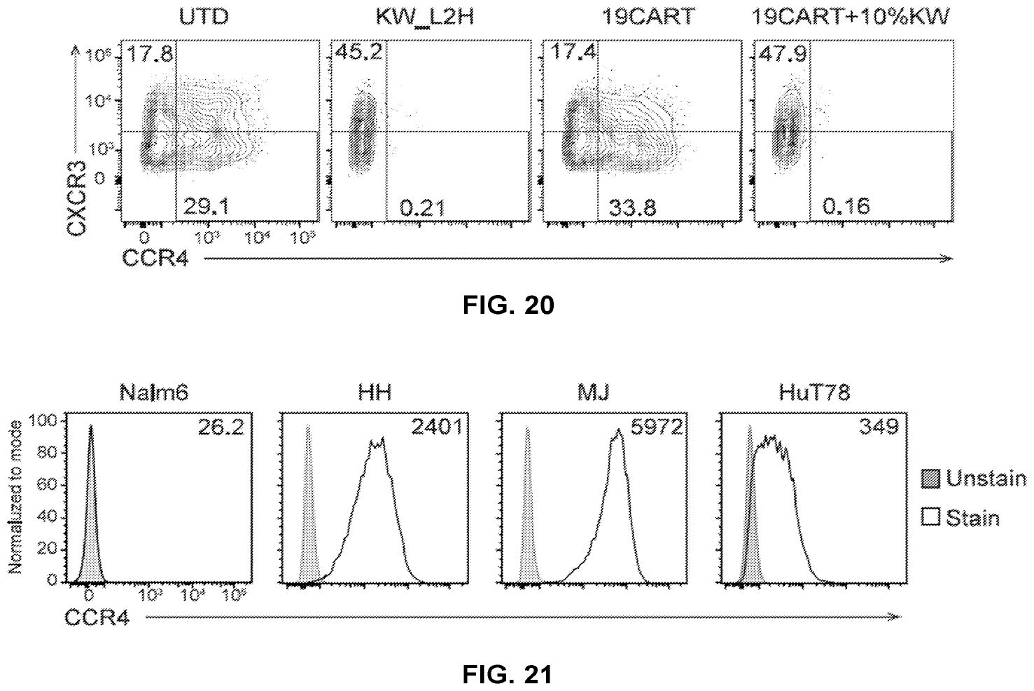

To investigate how CCR4-CAR T cell fratricide impact the distribution of CD4+ T cell subsets in CCR4-CAR T cell products, the levels of Th1, Th2, Th17 and Treg subsets and cytokine profiles were measured. It was observed that, compared to CD19-CAR T cells, CCR4-CAR T cell products contained a significantly decreased percentage of CD4+Th2 subset as defined by the following flow cytometry signature; CD4(+) CCR4(−) CXCR3(+) CCR6(−) CCR10(−), but maintained the level of CD4+Th1 subset; CD4(+) CCR4(−) CXCR3(+) CCR6(−) and CCR10(−) (FIG. 17). Treg subsets determined by FoxP3 and CD25 was rare and there was no difference between CD19-CAR T cell and CCR4-CAR T cell products (data not shown). In addition, upon stimulation with PMA-Ionomycin CCR4-CAR T cells produced decreased levels of Th2, Th17 and T-reg related cytokines while maintaining a Th1-related cytokine, INF-γ, and proinflammatory cytokine (TNF-α and IL-2) production (FIG. 18). These results suggested that transduction of T cells with CCR4-CAR results in fratricidal depletion of CCR4-positive Th2 and Th17 and Treg subsets and maintains CCR4-negative Th1 CD4+ subset. This depletion may be beneficial for other CAR T cell product by changing Th subset profiles. To address this, CCR4-CAR T cells were utilized to make Th1 rich CD19-CAR T cell product. Adding of just 10% of CCR4-CAR T cells depleted total CCR4 positive T cell subsets in CD19-CAR T cells (FIG. 19) resulting in relatively Th1 dominant CD19-CAR T cell product (FIG. 20).

### Example 5: CCR4-CAR T Cells Respond to and Lyse CCR4 Positive T Cell Tumor Cell Lines In Vitro

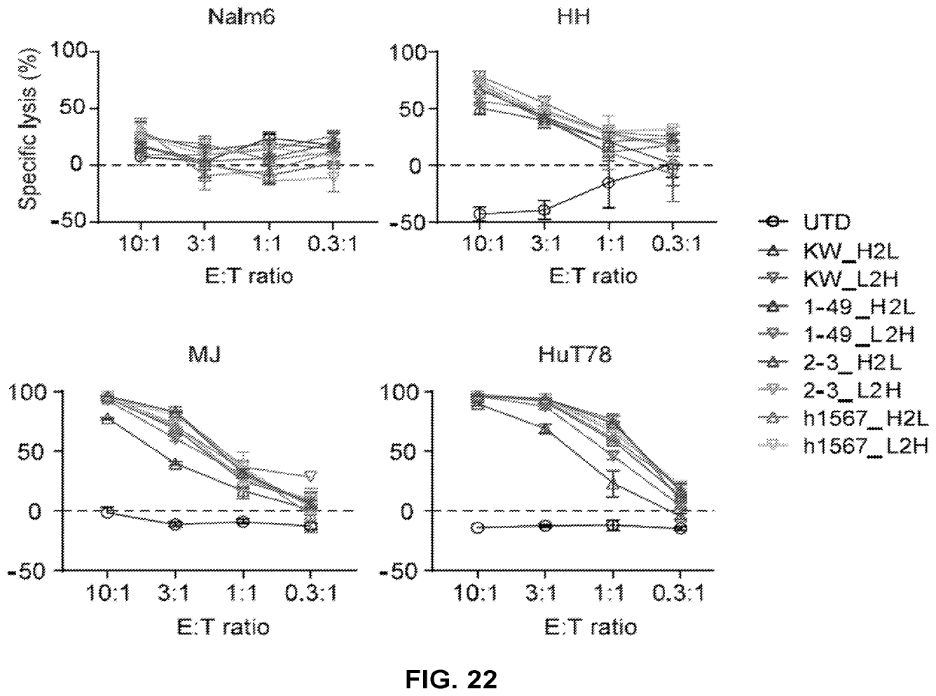

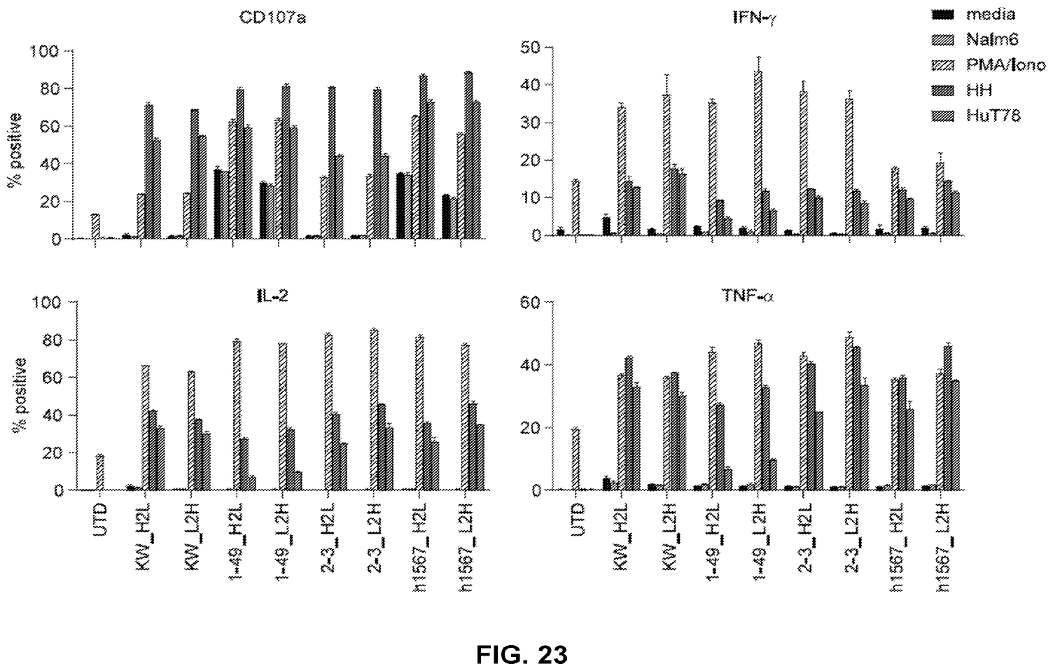

Since target antigen density impacts CAR T cell responses, the lytic activity of the panel of CCR4-CAR T cells were tested against three cell lines derived from patients with T cell malignancies displaying a range of CCR4 expression (FIG. 21). All of the CCR4-CAR T cells lysed the CCR4-expressing cells lines. No difference in lytic activity was observed between the CCR4-CAR T cells against HH, the highest CCR4-expressing cell line. In both the intermediate and low CCR4-expressing cell lines, MJ and HuT78 respectively, CCR4(KW_H2L/L2H)-CAR T cells exhibited the highest killing capacity and CCR4(1-49_H2L/L2H)-CAR T cells had the lowest (FIG. 22). All of the CCR4-CAR T cells degranulated upon CCR4 stimulation as reflected by CD107a upregulation, however anti-CCR4(1-49_H2L/L2H)-CAR T cells and CCR4(h1567_H2L/L2H)-CAR T cells showed target cell-independent degranulation at base line (FIG. 23). All the CCR4-CAR T cells secreted IFN-γ, IL-2 and TNF-α upon antigen independent-stimulation with PMA/Ionomycin.

### Example 6: CCR4-CAR T Cells Eradicate Tumor Cells in HH Cell Xenograft Mouse Model

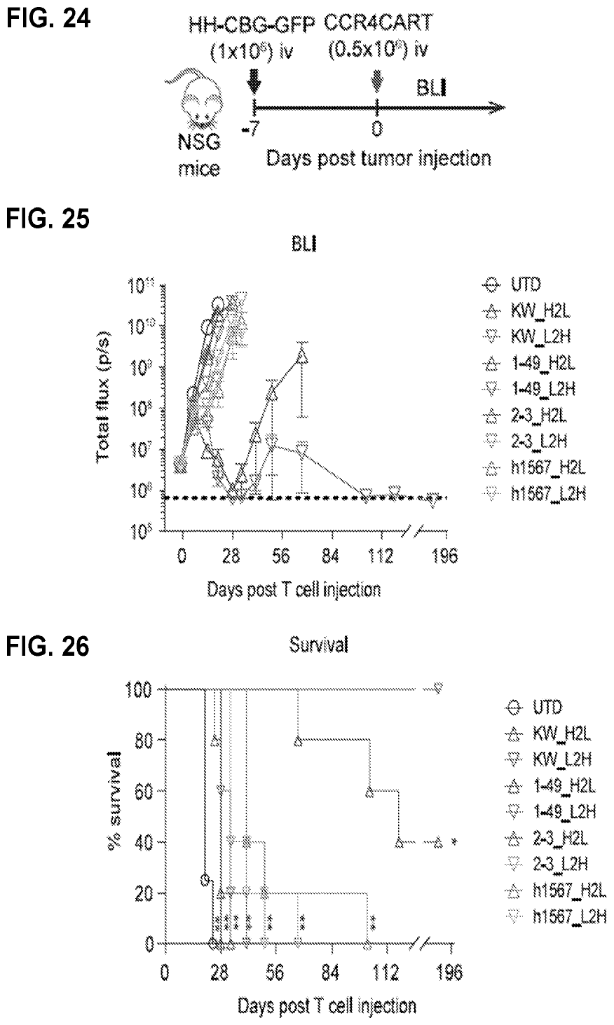

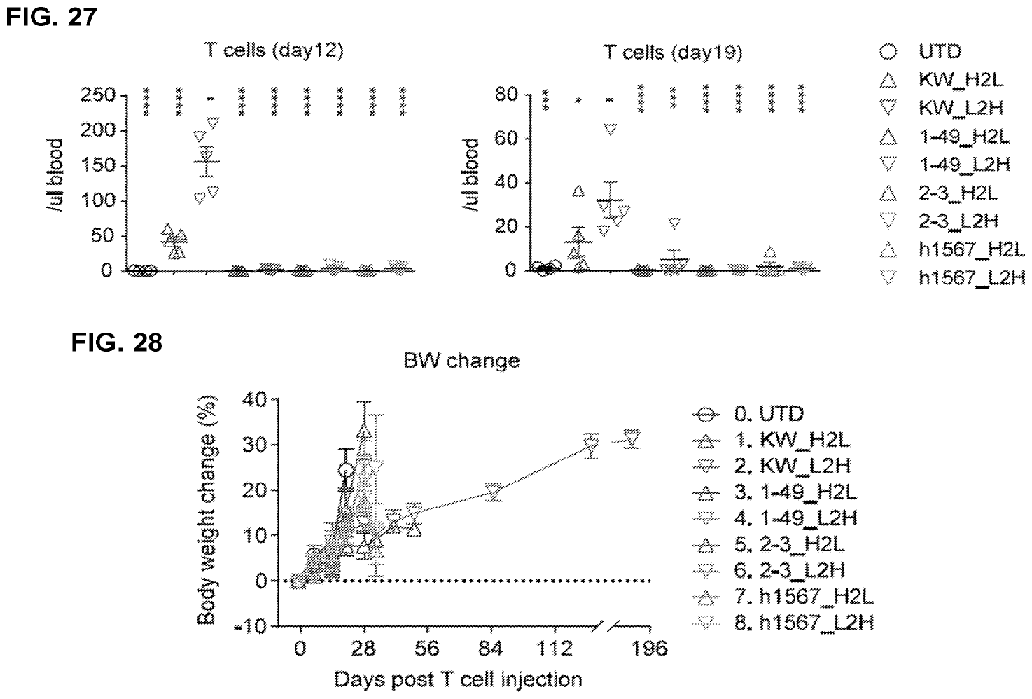

To compare the anti-tumor efficacy of CCR4-CARs in vivo, HH cell xenograft mice were treated with a low dose (0.5×10⁶) of CAR positive T cells or control UTD T cells to highlight any differences in effectiveness (FIG. 24). Tumor burden was followed by BLI for over 6 months to assess long-term remission. CCR4(KW_H2L/L2H)-CAR T cells showed superior anti-tumor efficacy and only CCR4(KW_L2H)-CAR T cells cured all mice (FIG. 25) with 100% survival benefit over the course of the experiment (FIG. 26). Superior anti-tumor efficacy was associated with better T cell engraftment; Anti-CCR4(KW_H2L/L2H)-CAR T cells induced robust T cell engraftment (FIG. 27). Additionally, CCR4(h1567_L2H)-CAR T cells exhibited mild anti-tumor efficacy and prolonged survival benefit. Apparent non-tumor related toxicity including GVHD and body weight loss were not observed throughout the experiment (FIG. 28). Based upon these results, CCR4(KW_L2H)-CAR T and CCR4(h1567_L2H)-CAR T cells were selected for further evaluation.

### Example 7: Antitumor Efficacy and T Cell Engraftment of CCR4-CAR T Cells are Comparable or Superior to CD19-CAR T Cells

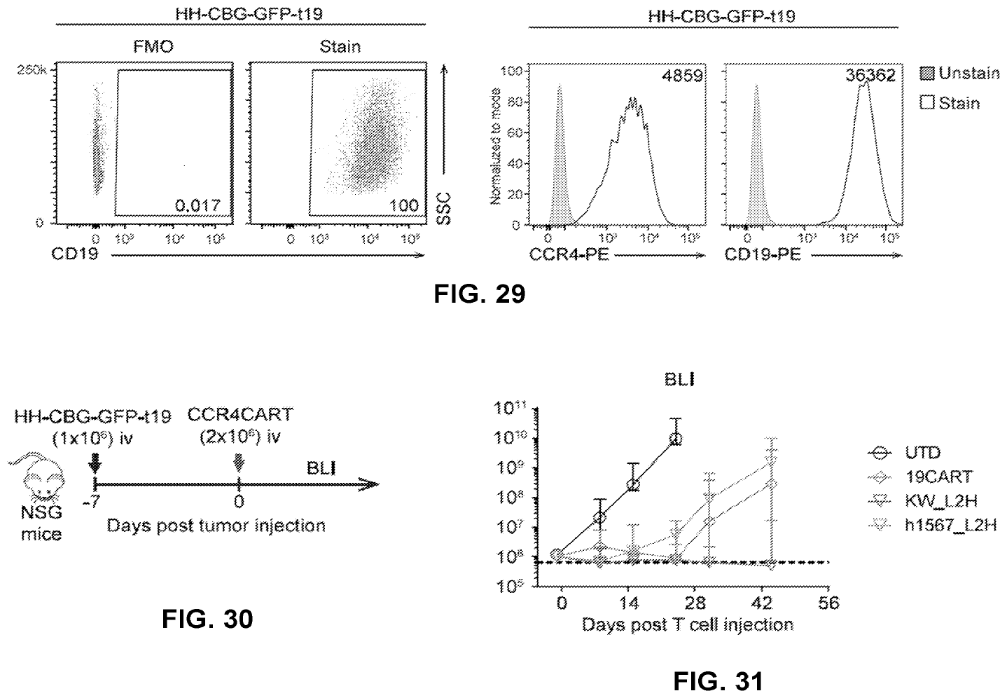

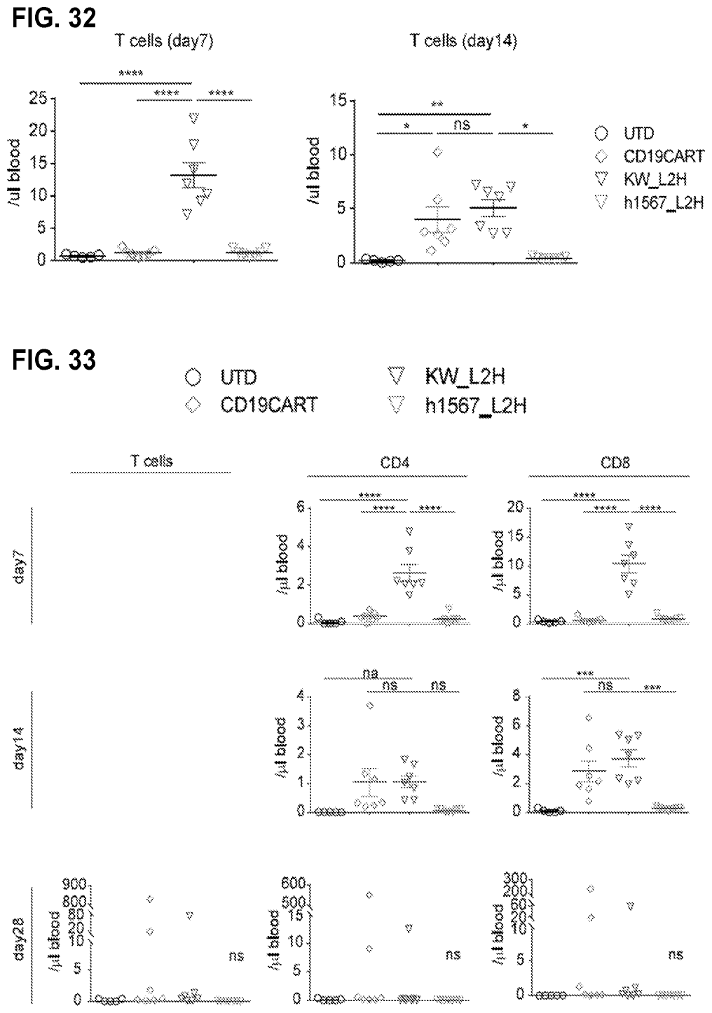

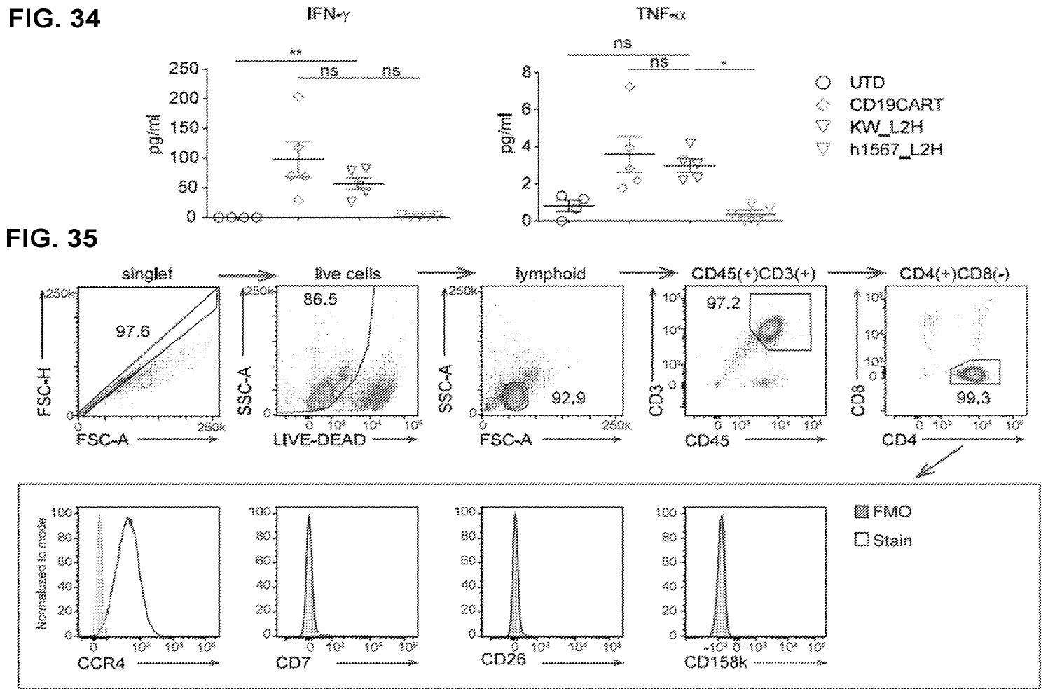

To further evaluate the in vivo efficacy of CCR4-CAR T cells, their ability to clear tumors expressing both CCR4 and CD19 was compared to that of validated anti-CD19-CAR T cells. HH cells were transduced with CD19 to obtain CCR4 and CD19 double-positive cells. Cells were sorted to achieve 100% expression of CD19 by FCM (HH-CBG-GFP-t19) (FIG. 29). NSG mice were inoculated with 1×10⁶ HH-CBG-GFP-t19 cells and treated with 2×10⁶ CAR positive T cells or UTD cells at day 7 after tumor inoculation (FIG. 30). CD19-CAR T cells and CCR4(h1567_L2H)-CAR T cells showed similar anti-tumor efficacy against CCR4 and CD19-expressing tumors, whereas CCR4(KW_L2H)-CAR T cells induced complete remission (FIG. 31). Additionally, CCR4(KW_L2H)-CAR T cells showed higher levels of T cell engraftment than CD19-CAR T cells in the earlier phase (day 7) and showed comparable T cell engraftment at the later phase (day14; FIG. 32 and FIG. 33). Very few T cells were detectable in peripheral blood at day 28 with exception of one mouse from the CD19-CAR T cell group that developed GVHD and showed rapid increase of T cells in peripheral blood (FIG. 33). CCR4(KW_L2H)-CAR T cells and CD19-CAR T cells produced comparable levels of IFN-γ and TNF-α but CCR4(h1567_L2H)-CAR T cells secreted lower levels of cytokines (FIG. 34). These results establish the in vitro and in vivo efficacy of the lead CCR4(KW_L2H)-CAR T cells against CCR4-expressing tumor lines.

### Example 8: CCR4-CAR T Cells Respond to and Lyse Primary CTCL Cells

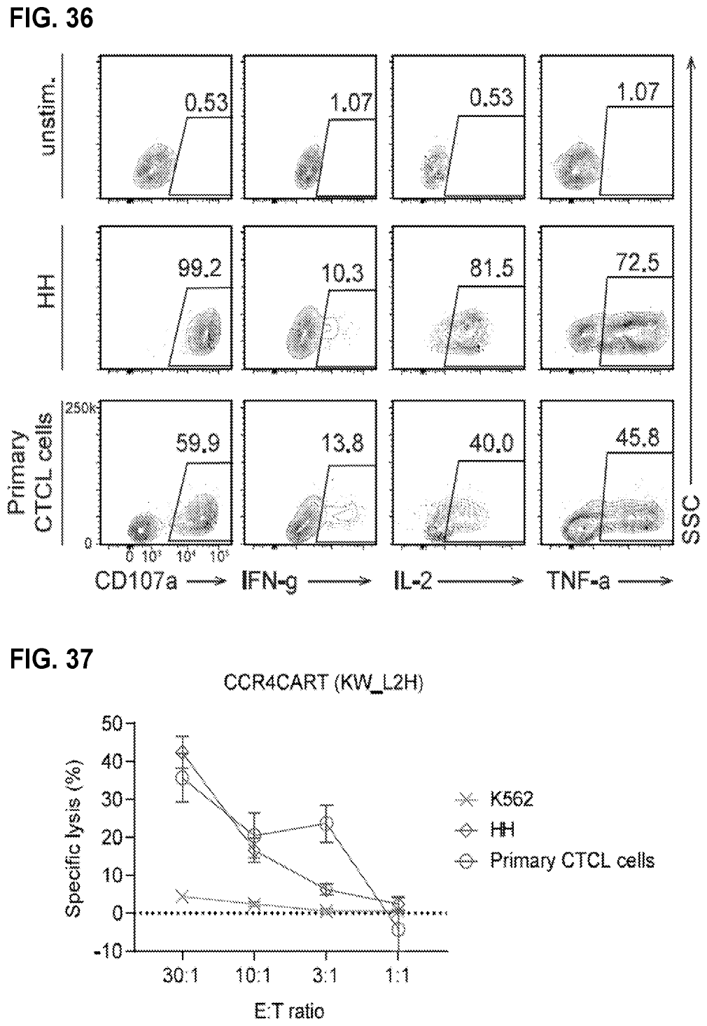

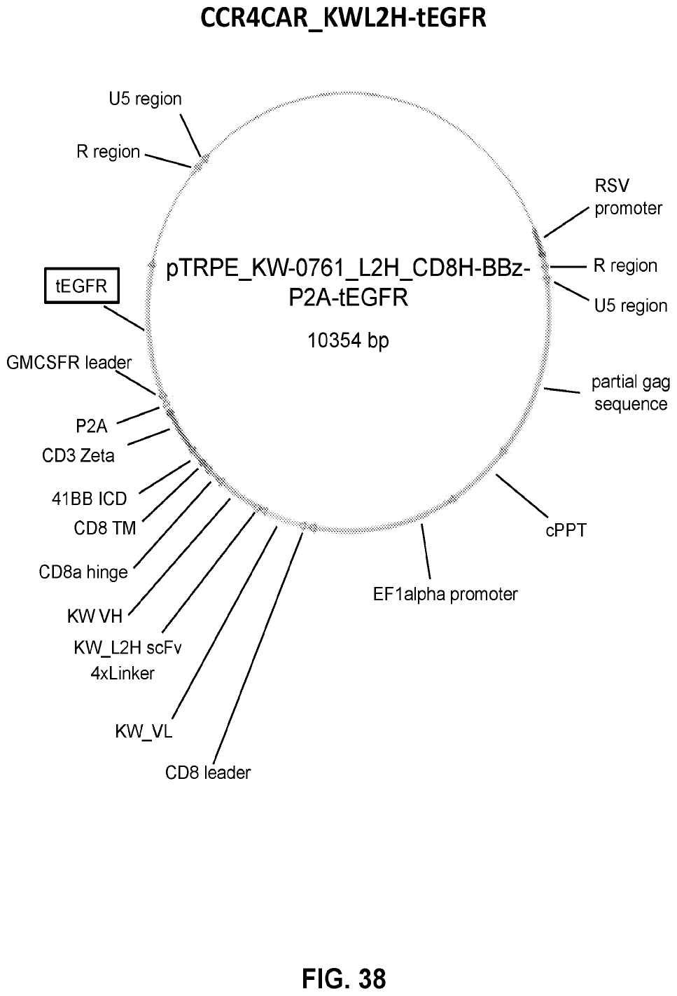

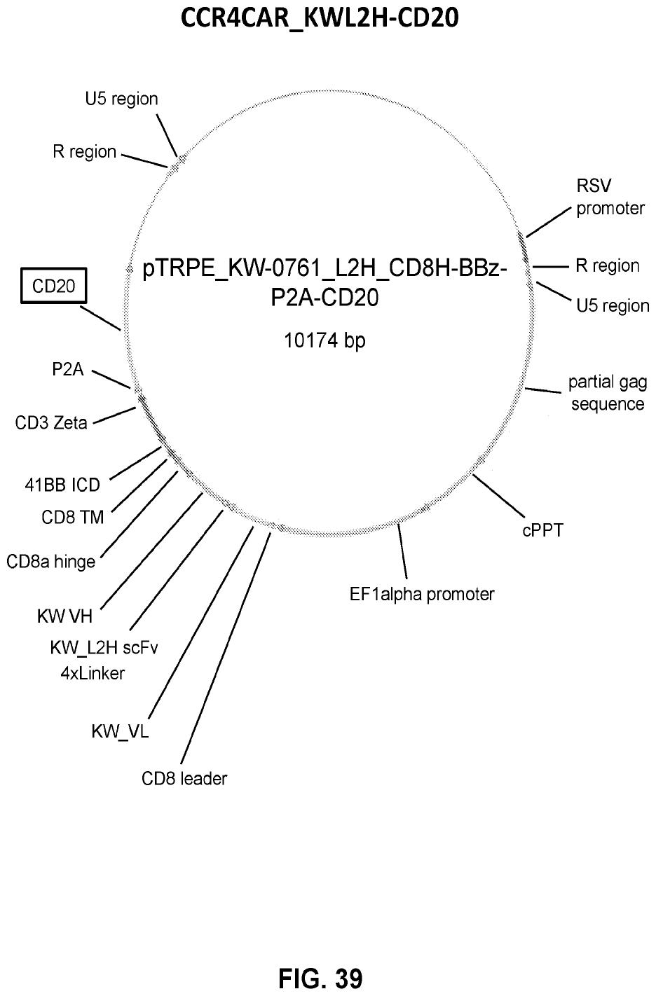

To validate that CCR4(KW_L2H) CAR T cells can target and kill T cell Lymphoma patient samples, their lytic activity against patient derived primary CTCL cells was tested. Initially, it was confirmed that isolated tumor cells expressed phenotypic markers characteristic of CTCL; CD4(+), CCR4(+), CD7(−), CD25(−), CD157(−) and also expressed CCR4 by flow cytometry32 (FIG. 35). In addition, it was confirmed that the patient sample contained a low frequency of normal cells (less than 5%. Upon co-culture with the CTCL patient sample, CCR4(KW_L2H)-CAR T cells were activated as demonstrated by CD107a upregulation and secreted IFN-γ, IL-2 and TNF-α cytokines (FIG. 36). Importantly, CCR4(KW_L2H)-CAR T cells specifically and effectively lysed CTCL patient tumor cells (FIG. 37).

### Example 9: Equipping Anti-CCR4 Chimeric Antigen Receptor T Cells with a T Cell-Depleting System

Anti-CCR4-CAR T cells (CCR4CART cells) effectively lysed target tumor cells as well as normal CCR4-expressing T cells including regulatory T cells (Tregs) and type 2 helper T cells (Th2). Depletion of these cell fractions could “enhance” the CCR4CART cell products by relatively increasing Th1 and CD8 T cells while complete loss of normal Tregs potentially cause severe autoimmune reaction when translated to the clinic. Reported case of Stevens-Johnson syndrome with high infiltration of CD8 T cells after mogamulizumab therapy may be associated with Treg depletion. Further, CCR4 expression in normal organs has been reported including kidney, lung and pancreas in some data bases, but fortunately its expression levels are low and severe organ specific adverse effects other than manageable platelet decrease are not reported in the clinical studies of mogamulizumab. Nonetheless, enhanced activity of the CCR4-CAR T cell therapy described herein may induce such toxicity. Therefore modulation of CCR4-CAR via a suicide system, strategies of chemotherapeutic or antibiotic depletion of CAR T cells, or inducible CAR expression are strategies which are contemplated herein.

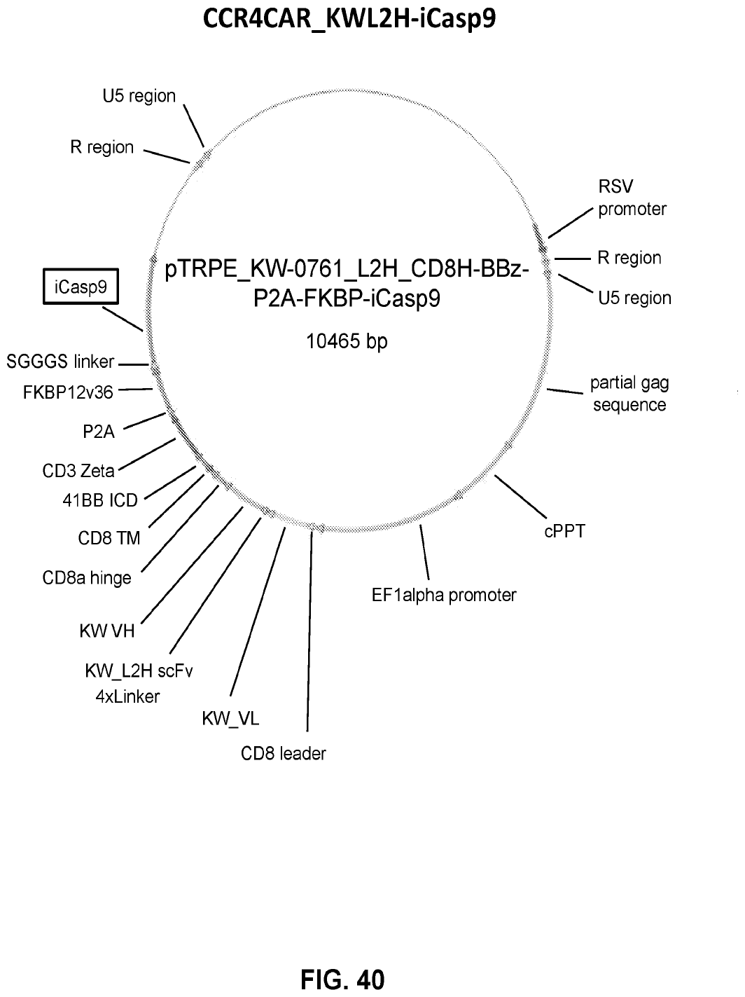

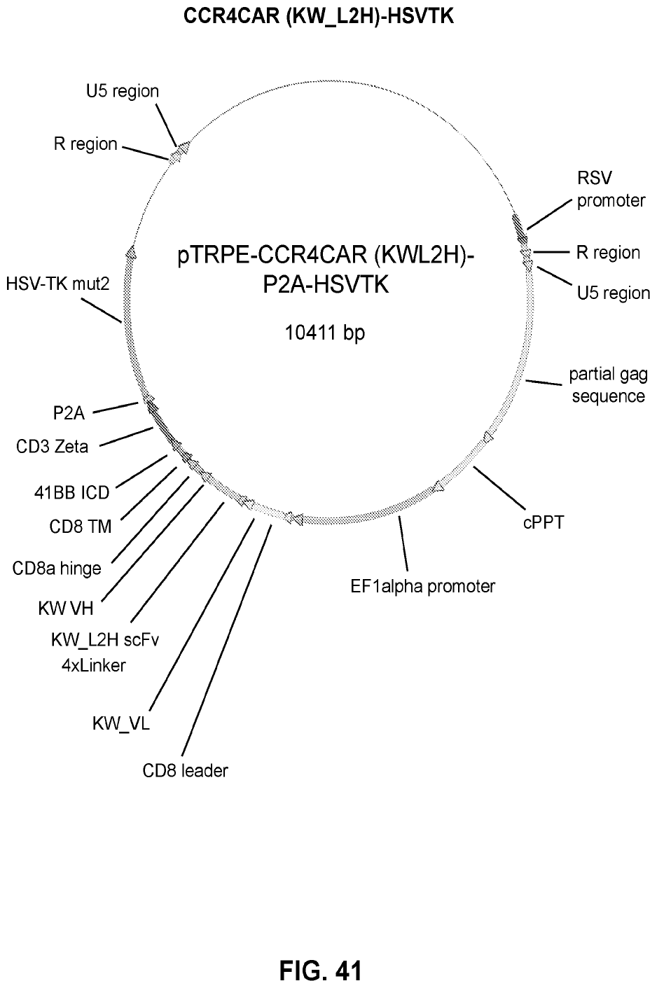

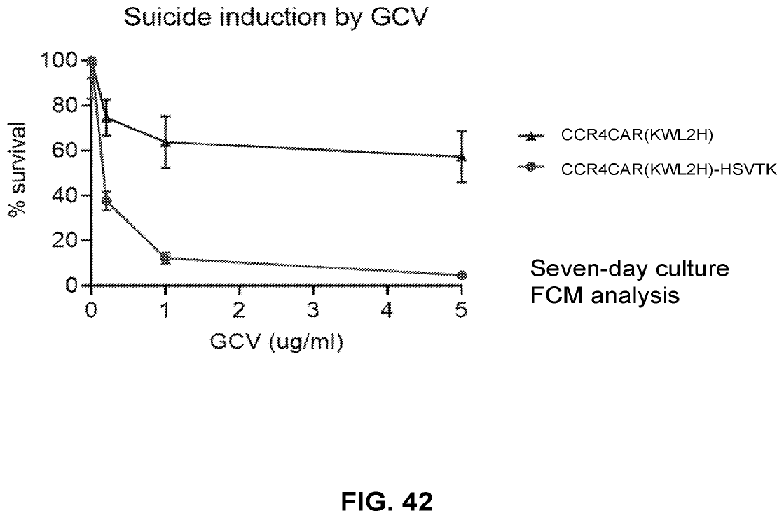

As a safety switch to shutdown CART cells when anti-CCR4 CAR T cell-mediated toxicity is observed and/or detected, the CCR4CAR(KW_L2H) lentiviral vector was further modified with a safety switch comprising truncated EGFR (EGFRt) (Michael C. Jensen et al. U.S. Pat. No. 8,802,374B2), CD20 (Sarah K. Tasian et al. Blood. 2017 Apr. 27; 129(17): 2395-2407), inducible caspase 9 (Karin C. Straathof et al. Blood. 2005 Jun. 1; 105(11): 4247-4254), or Herpes simplex virus-1 thymidine kinase (HSVTK) (Science. 1997 Jun. 13; 276(5319):1719-24) via P2A self-cleaving sequences (FIG. 38-FIG. 41). Each of these systems is expected to induce CART cell depletion upon administration of the corresponding safety switch agent, i.e., anti-EGFR monoclonal antibodies (mAb) (e.g. cetuximab), anti-CD20 mAbs (e.g. rituximab), a dimerizing drug (AP1903), and ganciclovir (GCV), respectively. Co-expression of tEGFR with CCR4CAR on T cells has been confirmed (data not shown). Additionally, using the HSVTK depletion system, CCR4CART cells are depleted by GCV in a dose dependent manner (FIG. 42).

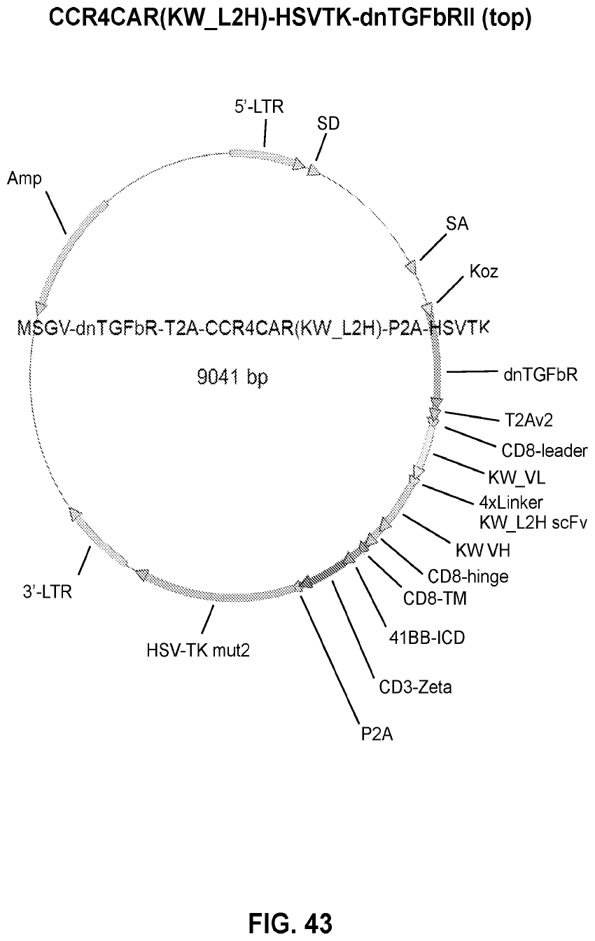

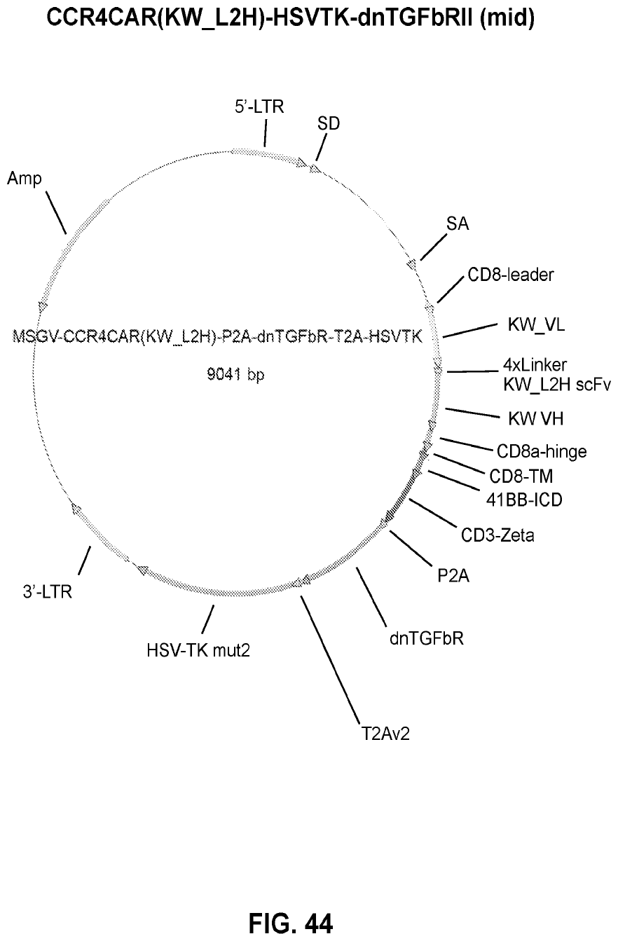

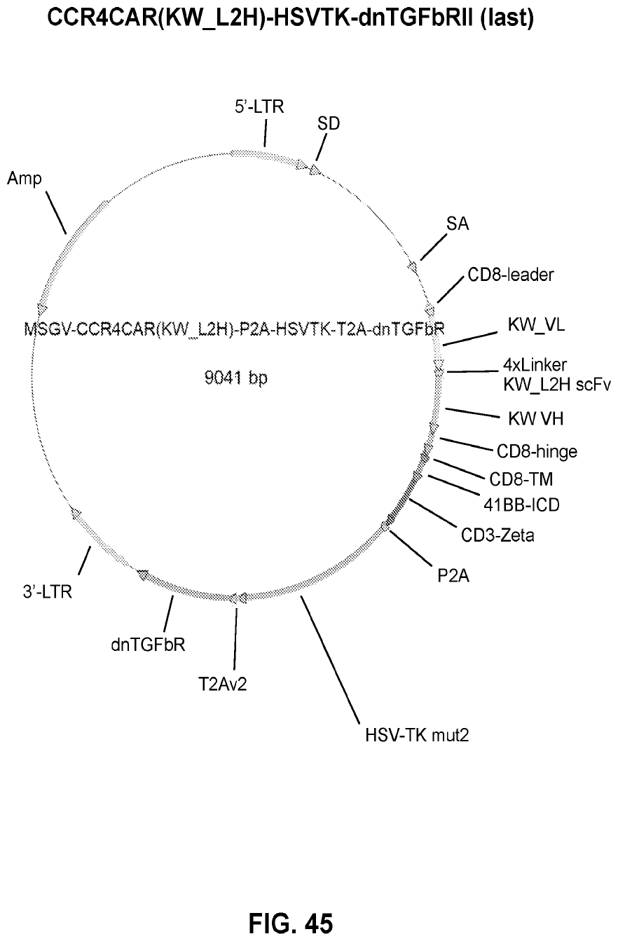

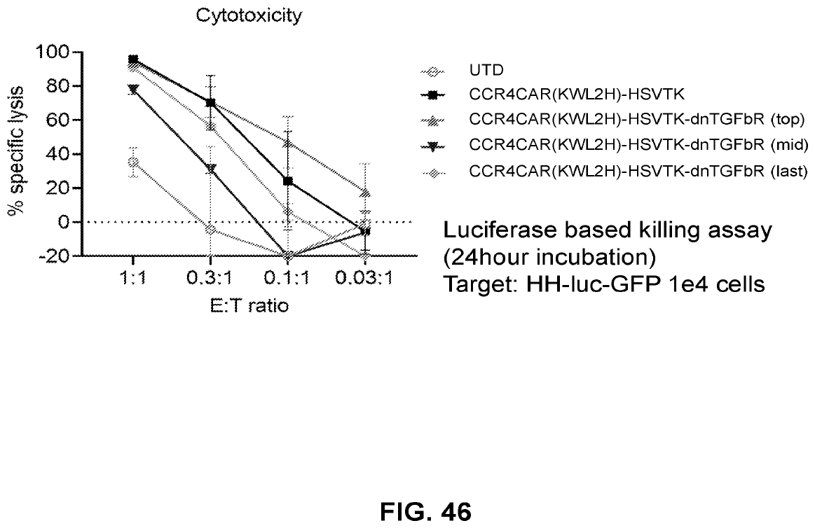

Some of tumors including ATLL create high TGFb tumor microenvironment (Blood (2011) 118 (7): 1865-1876.), which can induce immune cell disfunction. In addition, TGFb induces CCR4 expression on T cells after anti-CD3 or anti-CD3/28 expression, which may result in undesirable fratricidal killing of CART cells. Thus, the anti-CCR4CAR (KW-L2H)-HSVTK plasmid vector was further modified by inserting a dominant-negative TGFb receptor type II (dnTGFbRII) which can block TGFb signaling (Proc Natl Acad Sci USA. 1997 Mar. 18; 94(6):2386-91.). Three CCR4CAR(KWL2H)-HSVTK-dnTGFbRII constructs with different order of CAR, HSVTK and dnTGFbRII were generated; dnTGFbRII-CCR4CAR(KW_L2H)-HSVTK (top), CCR4CAR(KW_L2H)-dnTGFbRII-HSVTK (mid) and CCR4CAR(KW_L2H)-HSVTK-dnTGFbRII (last) (FIG. 43-FIG. 45). These vectors similarly produced cytokines (data not shown) and dnTGFbRII-CCR4CAR(KW_L2H)-HSVTK (top) had a trend inducing highest killing activity (FIG. 46).

Combining these systems with CCR4-CAR can overcome the most critical problem for CCR4-CAR T cell therapy, i.e., off-tumor toxicity, and enhance its feasibility to the clinical use. Also, ATLL that is a target tumor of the CCR4CART cells produce high levels of TGFb. Blocking TGFbRII can rescue CART cells from disfunction in tumor microenvironment. In addition, blocking TGFb signaling can prevent CCR4 overexpression, which can avoid undesirable fratricidal loss of effective CART cells.

### Example 10: Knocking Down CCR4 to Prepare CCR4-CAR T Cell Product without Fratricidal Events (CCR4 k/d CCR4-CAR T Cells)

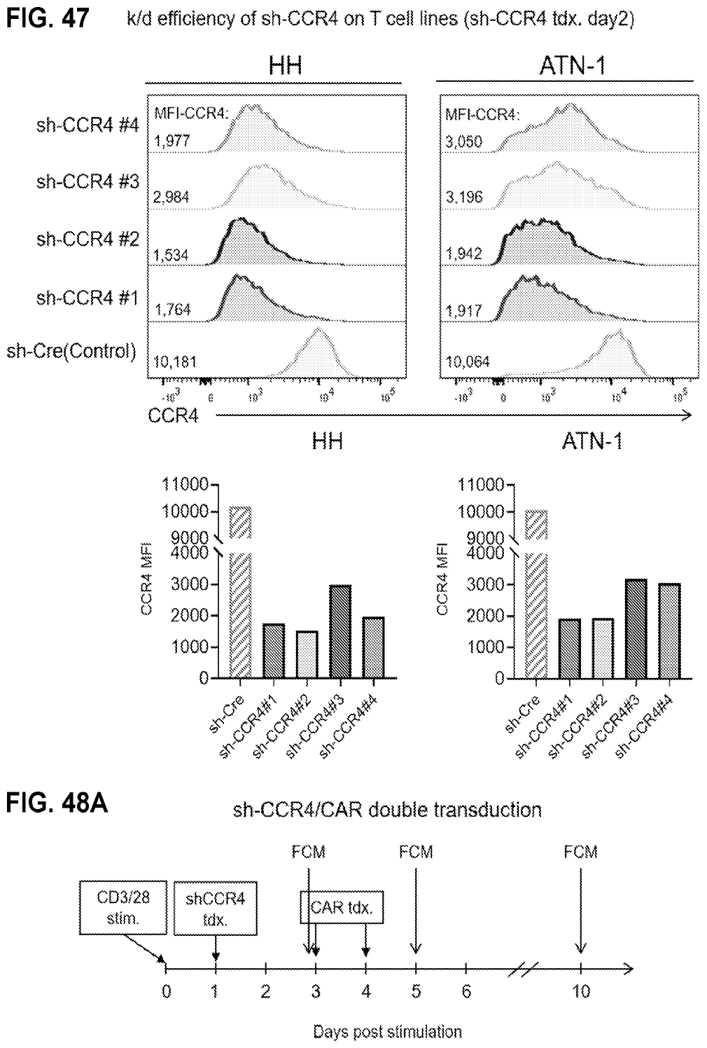

As demonstrated herein, CCR4-CAR T cells mutually kill and finally eradicate CCR4 positive T cells in the CAR T cell product leading to enhanced CCR4-CAR T cell production efficiency and functions. A further approach contemplated herein for reducing or eliminating fratricide of CCR4 expressing T cells involves knock down (k/d) or knock out (k/o) of CCR4 in the cells prior to CCR4-CAR transduction. This can be achieved by any method known in the art, such as by additional modification of T cells with RNAi (shRNA or siRNA), genetic engineering (for example, via CRISPR/Cas9), or with protein-based systems (e.g. anti-CCR4 scFvs with C-terminal KDEL motif fusions). Toward this end, four CCR4-targeting shRNAs (sh-CCR4s) were constructed, as well a Cre-targeting shRNA (sh-Cre) as a control (Table 4). The sh-Cre or sh-CCR4 shRNAs were cloned into pLVSIN-mU6-MCS-PGK-Neo plasmid using EcaRI and BamHI sites. The shRNAs were tested in CCR4 expressing cell lines HH and ATN-1 (FIG. 47). All sh-CCR4 could induce CCR4 knock down with varying levels of efficiency; sh-CCR4 #1 and #2 similarly induced efficient CCR4 k/d while sh-CCR4 #4 induced modest CCR4 k/d and CCR4 #3 showed mild CCR4 k/d.

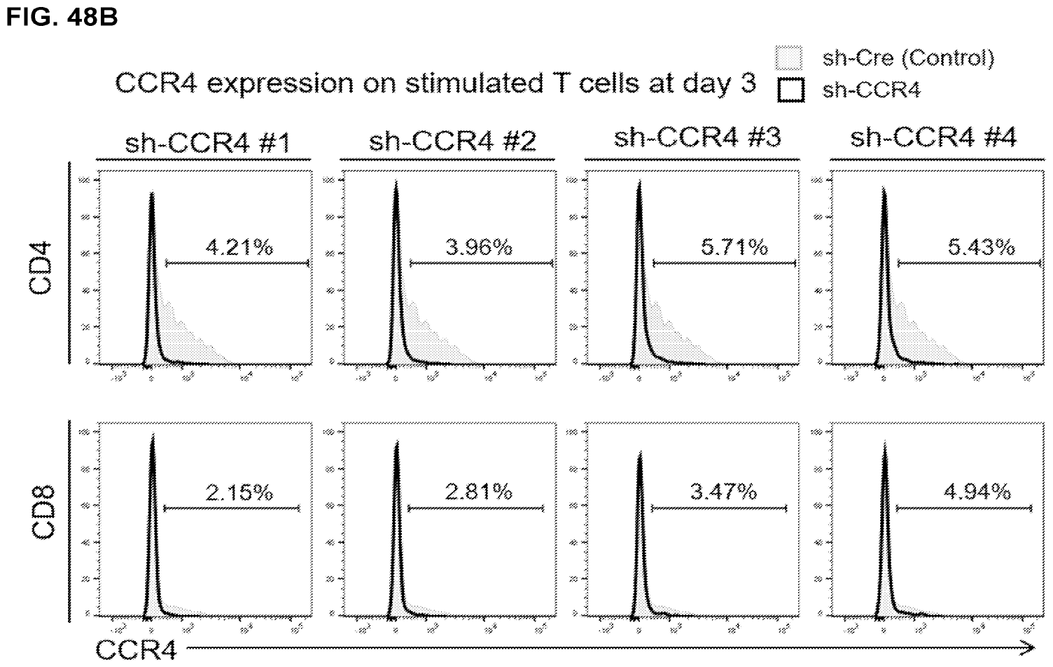

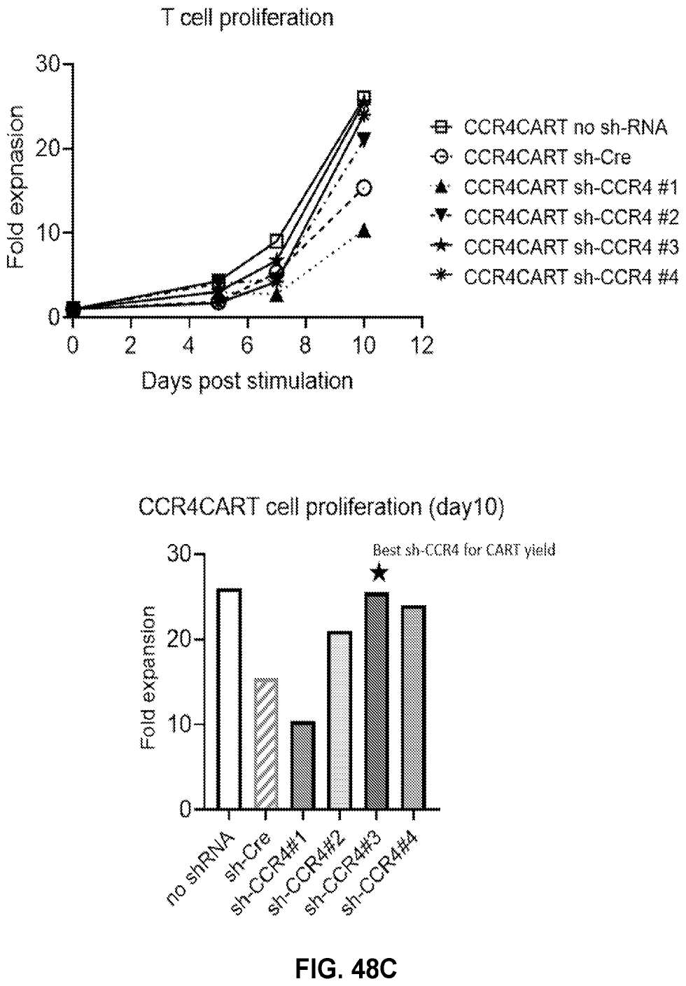

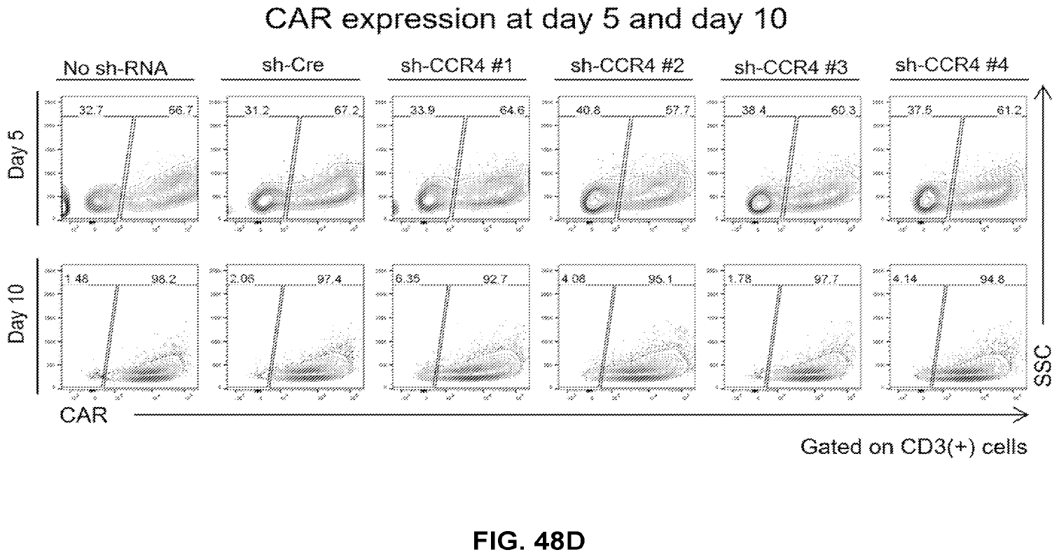

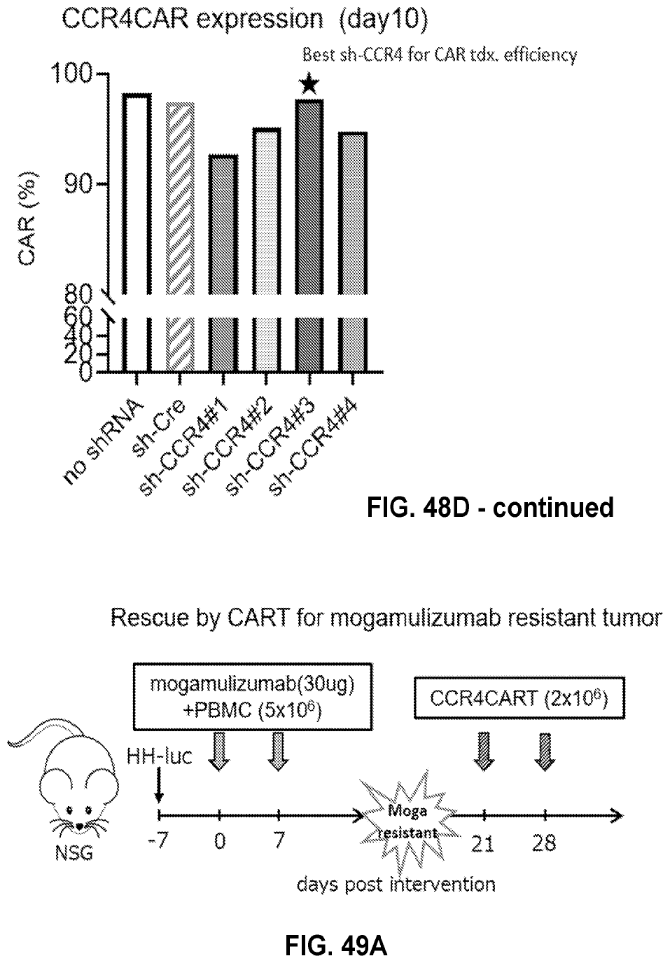

To establish CCR4-CAR T cells with minimal or no fratricidal events, primary T cells were transduced with the sh-CCR4s for CCR4 k/d at day 1 and then transduced with CCR4CAR(KW_L2H) at day 3 and 4 post anti-CD3/28 beads stimulation (FIG. 48A). Similar to the efficiency on the cell lines, any shCCR4s induced CCR4 k/d but with varying efficiency; sh-CCR4 #1 and #2 similarly induced most efficient CCR4 k/d while sh-CCR4 #4 induced modest CCR4 k/d and CCR4 #3 showed mild CCR4 k/d (FIG. 48B).

Importantly, the most efficient T cell proliferation and CAR transduction were achieved in the CAR T cells not transduced with a shCCR4, and similar efficiency was achieved for CAR T cells transduced with the least efficient shCCR4 (shCCR4 #3) (FIG. 48C and FIG. 48D). In addition, the most efficient sh-CCR4 (sh-CCR4 #1) for CCR4 k/d rather impaired efficient T cell proliferation and CAR transduction.

These results confirm that fratricidal events (mutual killing of CCR4 positive cells in CCR4-CAR T cell product) is beneficial for CCR4-CAR T production. Nonetheless, careful selection of sh-CCR4 construct can achieve CAR transduction and T cell proliferation efficiency levels comparable to those of the original CCR4-CAR T cell product.

### Example 11: CCR4-CAR T Cells (KW_L2H) can Eradicate Mogamulizumab Resistant T Cell Tumors In Vivo

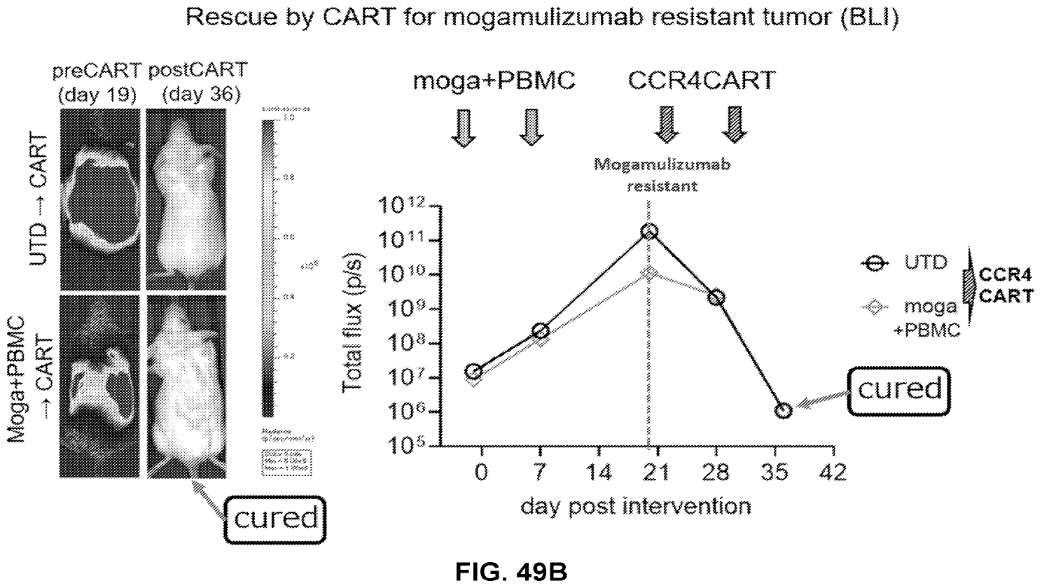

In clinical settings, CCR4-CAR T cells will be administrated in patient with refractory or relapsed disease after mogamulizumab treatment. A concern is epitope blocking by prior mAb therapy as CCR4-CAR T cells and mogamulizumab share its epitope on the CCR4 antigen. To address this, CCR4-CAR T cells were tested in a mogamulizumab refractory T cell lymphoma mouse model (FIG. 49A). HH tumors in NSC mice were treated with two doses of mogamulizumab in combination with PBMCs, which resulted in refractory disease. The refractory tumors were subsequently treated with CCR4-CAR (KW_L2H) T cells. CCR4-CAR T cells effectively suppressed and eradicated the tumor in the similar way against tumors without prior mogamulizumab treatment, suggesting the feasibility of the CCR4-CAR T cell therapy for mogamulizumab pre-treated and refractory tumors (FIG. 49B).

### Example 12: CCR4-CAR T Cells can be Safely and Efficiently Established from Patient-Derived PBMCs Highly Contaminated with Tumor Cells

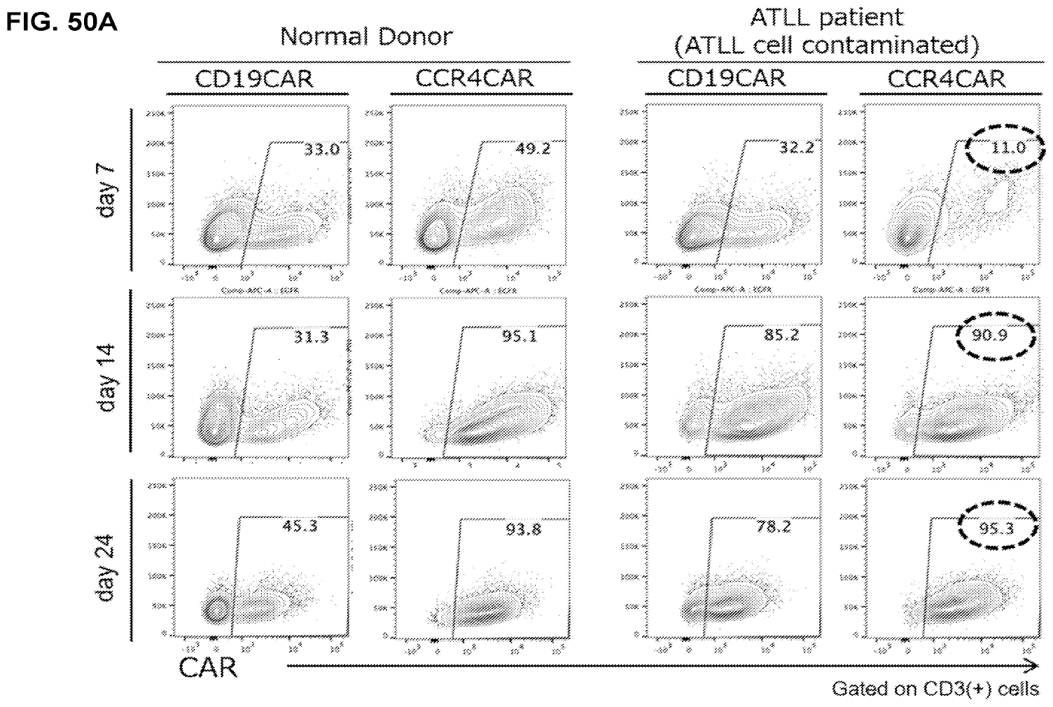

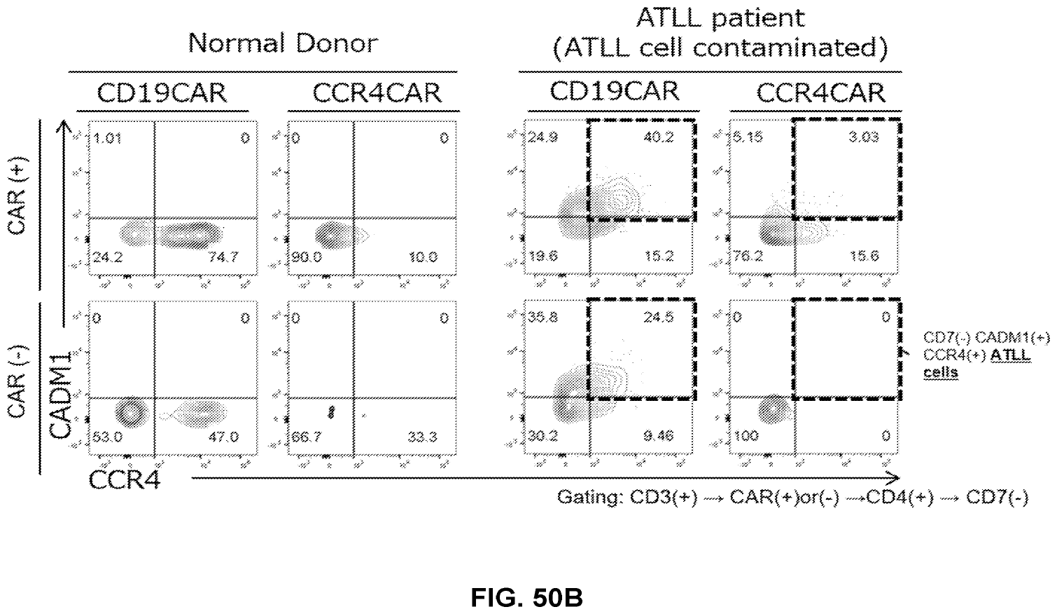

ATLL patients are typically heavily treated and PBMCs from these patients are contaminated with tumor cells. To test if CCR4-CAR T cells can be produced from those patients, PBMCs were collected from a patient with ATLL (70% of blood cells were tumor cells) and CART production was tested. PBMCs from the patient and a normal donor were transduced with either CD19CAR or CCR4CAR(KWL2H) at day 1 post anti-CD3/28 stimulation. Patient-derived PBMCs showed less transduction efficacy compared to those from normal donor, but eventually the percentage of CCR4-CAR positive cells increased and reached over 90% in both the patient and normal-donor derived products (FIG. 50A). In addition, contaminated CD7(−), CADM1(+) and CCR4(+) tumor cells were almost eradicated in CCR4-CAR T cell product but not in CD19-CAR T cell product (3.03% v.s 40.2% in CAR positive T cell population and 0% vs 24.5% in CAR negative T cell population) (FIG. 50B). This result confirms that CCR4-CAR T cells can be safely and efficiently established from patient-derived PBMCs which are highly contaminated with tumor cells.

### Example 13: CCR4-CAR T Cells for Treating T Cell Malignancies and Solid Tumors In Vivo

Mature T cell lymphomas including CTCL, ATLL and PTCL are generally associated with poor prognosis. Although new therapies such as anti-CCR4 mAb (mogamulizumab) can delay tumor progression, curative treatment is rarely achieved. The present work demonstrates mogamulizumab derived CCR4-CAR T cells can effectively target both CCR4 positive tumor cell lines and primary CTCL cells and can eradicate established tumors in preclinical models, which was superior to the previously reported h1567 and mAb2-3 derived CAR T cells (Perera L P, et al. Am J Hematol. 2017; 92(9):892-901). The present work also demonstrates mechanisms of fratricidal events and its relevance to CAR T cell functions; superior anti-tumor efficacy of CCR4-CAR T cells are associated with rapid and complete fratricidal depletion of CCR4 positive T cells. Additionally, data presented herein reveal that the degree of fratricide is scFv dependent, influencing the degree to which the CCR4-CAR can mask target CCR4 in cis. KW-0761 only partly binds CCR4 in cis permitting unhindered fratricide, but CARs derived from h1567 and its derivatives almost completely block the epitope in cis allowing CCR4-CAR positive cells to persist. This observation is compatible with a previous report that CD19-CAR unintentionally introduced into a leukemic B cell induced a leukemia resistance by binding to CD19 epitope on the surface of leukemic cells, masking it from recognition by CD19-CAR T cells.

It is contemplated herein that Th1 dominant CAR T cell product relative to Th2 and T regs will enhance anti-tumor activity of CART cells as Th1 subsets play central roles in anti-cancer immunity whereas Th2 and Tregs possibly counteract against it. CD19-CAR T cells achieved such “enhanced” CAR T cell product by mixing with small amount of CCR4 CAR T cells, which indicates that CCR4-CAR T cells can be utilized as a tool to improve other CAR T cell products. This result also indicates that host normal CCR4 positive T cells can be depleted by CCR4-CAR T cells like shown in mogamulizumab treatment. Th2-mediated inflammation is often beneficial for tumor growth and cancer immune evasion, and higher Th2 infiltration is associated with poor treatment outcome. Tregs also have an important role for tumor growth and immune evasion. Tregs can suppress effector T cell mediated tumor killing and high Treg infiltration to TME is associated with poor outcome of many types of tumors including colorectal, breast, ovarian, renal, non-small cell lung, melanoma, lymphoma, and hepatocellular cancers. Even transient depletion of T regs can enhance the efficacy of tumor vaccine therapy in animal models. Therefore, it is contemplated herein that depletion of CCR4 positive Treg and Th2 by CCR4-CAR T cells will contribute to the anti-tumor immunity for cancer in general in addition to direct killing of CCR4 positive tumor cells. Indeed, mogamulizumab has been tested as a Treg depleting therapy in solid tumor patients. Significant reduction of CCR4 positive T cells and immune responses to cancer/testis (CT) antigens and an autoantibody response to thyroid peroxidase were observed following mogamulizumab in some patients.

In summary, the present work identifies highly active CCR4-CAR T cells derived from KW-0761(mogamulizumab). Efficacy was shown to be associated with fratricidal depletion of Th2, Treg and Th17 subsets (all CCR4 positive) while sparing Th1 subset (CCR4 negative). It is further contemplated herein that CCR4-CAR T cell format will be beneficial for the treatment of patients with T cell malignancies as well as for a Th2 and Treg depleting therapy for treating other cancers including solid tumors.

### Enumerated Embodiments

The following enumerated embodiments are provided, the numbering of which is not to be construed as designating levels of importance.

Embodiment 1 provides an anti-CC chemokine receptor 4 (CCR4) chimeric antigen receptor (CAR) comprising an anti-CCR4 antigen binding domain, a transmembrane domain, and an intracellular domain.

Embodiment 2 provides the CAR of embodiment 1, wherein the anti-CCR4 antigen binding domain comprises at least one heavy chain variable region (HCDR) comprising the amino acid sequence selected from the group consisting of SEQ ID NO: 2, SEQ ID NO: 4, and SEQ ID NO: 6.

Embodiment 3 provides the CAR of embodiment 1 or embodiment 2, wherein the anti-CCR4 antigen binding domain comprises at least one light chain variable region (LCDR) comprising the amino acid sequence selected from the group consisting of SEQ ID NO: 8, SEQ ID NO: 10, and SEQ ID NO: 12.

Embodiment 4 provides the CAR of any one of embodiments 1-3, wherein the anti-CCR4 antigen binding domain is selected from the group consisting of a full length antibody or antigen-binding fragment thereof, a Fab, a single-chain variable fragment (scFv), or a single-domain antibody.

Embodiment 5 provides the CAR of any one of embodiments 1-4, wherein the anti-CCR4 antigen binding domain is an anti-CCR4 single-chain variable fragment (scFv).

Embodiment 6 provides the CAR of any one of embodiments 1-5, wherein the anti-CCR4 antigen binding domain is an anti-CCR4 single-chain variable fragment (scFv) having a heavy chain and a light chain derived from mogamulizumab.

Embodiment 7 provides the CAR of any one of embodiments 1-6, wherein the anti-CCR4 antigen binding domain comprises a heavy chain variable region comprising an amino acid sequence at least 80%, 85%, 90%, 95%, 96%, 97%, 98%, 99%, or 100% identical to SEQ ID NO: 112.

Embodiment 8 provides the CAR of any one of embodiments 1-7, wherein the anti-CCR4 antigen binding domain comprises a light chain variable region comprising an amino acid sequence at least 80%, 85%, 90%, 95%, 96%, 97%, 98%, 99%, or 100% identical to SEQ ID NO: 114.

Embodiment 9 provides the CAR of any one of embodiments 1-8, wherein the anti-CCR4 antigen binding domain comprises an amino acid sequence at least 80%, 85%, 90%, 95%, 96%, 97%, 98%, 99%, or 100% identical to SEQ ID NO: 16.

Embodiment 10 provides the CAR of any one of the preceding embodiments, wherein the CAR further comprises a CD8 alpha hinge, optionally wherein the CD8 alpha hinge comprises the amino acid sequence set forth in SEQ ID NO: 34.

Embodiment 11 provides the CAR of any one of the preceding embodiments, wherein the transmembrane domain comprises a transmembrane domain selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS, and CD154, or a transmembrane domain derived from a killer immunoglobulin-like receptor (KIR).

Embodiment 12 provides the CAR of any one of the preceding embodiments, wherein the transmembrane domain comprises a CD8 alpha transmembrane domain, optionally wherein the CD8 alpha transmembrane domain comprises the amino acid sequence set forth in SEQ ID NO:

36.

Embodiment 13 provides the CAR of any one of the preceding embodiments, wherein the intracellular domain comprises a costimulatory domain and an intracellular signaling domain.

Embodiment 14 provides the CAR of any one of the preceding embodiments, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 15 provides the CAR of any one of the preceding embodiments, wherein the intracellular domain comprises a costimulatory domain of 4-1BB optionally comprising the amino acid sequence set forth in SEQ ID NO: 38.

Embodiment 16 provides the CAR of any one of the preceding embodiments, wherein the intracellular domain comprises an intracellular signaling domain of a protein selected from the group consisting of a human CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 17 provides the CAR of any one of the preceding embodiments, wherein the intracellular signaling domain comprises an intracellular signaling domain of CD3 or a variant thereof, optionally comprising the amino acid sequence set forth in SEQ ID NO: 40.

Embodiment 18 provides the CAR of any one of the preceding embodiments, wherein the CAR comprises an anti-CCR4 antigen binding domain comprising an anti-CCR4 scFv, a CD8 alpha hinge, a CD8 alpha transmembrane domain, an intracellular domain comprising a 4-1BB costimulatory domain and a CD3 intracellular signaling domain.

Embodiment 19 provides the CAR of embodiment 18, wherein the CAR further comprises a CD8 leader sequence, optionally comprising the amino acid sequence set forth in SEQ ID NO: 32.

Embodiment 20 provides the CAR of any one of the preceding embodiments, wherein the CAR comprises an amino acid sequence at least 80%, 85%, 90%, 95%, 96%, 97%, 98%, 99%, or 100% identical to any one of SEQ ID NOs: 30, 54, 62, 70, 82, 90, and 102.

Embodiment 21 provides the CAR of any one of the preceding embodiments, wherein the CAR is encoded by a nucleotide sequence at least 80%, 85%, 90%, 95%, 96%, 97%, 98%, 99%, or 100% identical to any one of SEQ ID NOs: 29, 53, 61, 69, 81, 89, and 101.

Embodiment 22 provides the CAR of any one of the preceding embodiments, wherein the CAR is linked to a safety switch via a self-cleavable linker, and wherein the safety switch binds a safety switch agent.

Embodiment 23 provides the CAR of embodiment 22, wherein the safety switch is selected from: (a) truncated EGFR (EGFRt), wherein the safety switch agent is an anti-EFGR antibody, optionally cetuximab; (b) CD20, wherein the safety switch agent is an anti-CD20 antibody, optionally rituximab; (c) inducible caspase 9 (iCasp9), wherein the safety switch agent is AP1903; and (d) Herpes simplex virus-1 thymidine kinase (HSVTK), wherein the safety switch agent is ganciclovir (GCV).

Embodiment 24 provides the CAR of embodiment 22 or embodiment 23, wherein the CAR is linked to a dominant negative TGFb receptor type II (dnTGFbRII) via a self-cleavable linker.

Embodiment 25 provides the CAR of embodiment 22 or embodiment 23, wherein the safety switch is linked to a dominant negative TGFb receptor type II (dnTGFbRII) via a self-cleavable linker.

Embodiment 26 provides a nucleic acid comprising a polynucleotide sequence encoding an anti-CCR4 CAR, wherein the CAR comprises an anti-CCR4 antigen binding domain, a transmembrane domain, and an intracellular domain.

Embodiment 27 provides the nucleic acid of embodiment 26, wherein the anti-CCR4 antigen binding domain comprises at least one heavy chain variable region (HCDR) comprising the amino acid sequence selected from the group consisting of SEQ ID NO: 2, SEQ ID NO: 4, and SEQ ID NO: 6.

Embodiment 28 provides the nucleic acid of embodiment 25 or embodiment 27, wherein the anti-CCR4 antigen binding domain comprises at least one light chain variable region (LCDR) comprising the amino acid sequence selected from the group consisting of SEQ ID NO: 8, SEQ ID NO: 10, and SEQ ID NO: 12.

Embodiment 29 provides the nucleic acid of any one of embodiments 26-28, wherein the anti-CCR4 antigen binding domain is selected from the group consisting of a full length antibody or antigen-binding fragment thereof, a Fab, a single-chain variable fragment (scFv), or a single-domain antibody.

Embodiment 30 provides the nucleic acid of any one of embodiments 26-29, wherein the anti-CCR4 antigen binding domain is an anti-CCR4 single-chain variable fragment (scFv).

Embodiment 31 provides the nucleic acid of any one of embodiments 26-30, wherein the anti-CCR4 antigen binding domain is an anti-CCR4 single-chain variable fragment (scFv) having a heavy chain and a light chain derived from mogamulizumab.

Embodiment 32 provides the nucleic acid of any one of embodiments 26-31, wherein the anti-CCR4 antigen binding domain comprises a heavy chain variable region comprising an amino acid sequence at least 80%, 85%, 90%, 95%, 96%, 97%, 98%, 99%, or 100% identical to SEQ ID NO: 112.

Embodiment 33 provides the nucleic acid of any one of embodiments 26-32, wherein the anti-CCR4 antigen binding domain comprises a light chain variable region comprising an amino acid sequence at least 80%, 85%, 90%, 95%, 96%, 97%, 98%, 99%, or 100% identical to SEQ ID NO: 114.

Embodiment 34 provides the nucleic acid of any one of embodiments 26-33, wherein the anti-CCR4 antigen binding domain comprises an amino acid sequence at least 80%, 85%, 90%, 95%, 96%, 97%, 98%, 99%, or 100% identical to SEQ ID NO: 16.

Embodiment 35 provides the nucleic acid of any one of the preceding embodiments, wherein the CAR further comprises a CD8 alpha hinge, optionally wherein the CD8 alpha hinge comprises the amino acid sequence set forth in SEQ ID NO: 34.

Embodiment 36 provides the nucleic acid of any one of the preceding embodiments, wherein the transmembrane domain comprises a transmembrane domain selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS, and CD154, or a transmembrane domain derived from a killer immunoglobulin-like receptor (KIR).

Embodiment 37 provides the nucleic acid of any one of the preceding embodiments, wherein the transmembrane domain comprises a CD8 alpha transmembrane domain, optionally wherein the CD8 alpha transmembrane domain comprises the amino acid sequence set forth in SEQ ID NO: 36.

Embodiment 38 provides the nucleic acid of any one of the preceding embodiments, wherein the intracellular domain comprises a costimulatory domain and an intracellular signaling domain.

Embodiment 39 provides the nucleic acid of any one of the preceding embodiments, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 40 provides the nucleic acid of any one of the preceding embodiments, wherein the intracellular domain comprises a costimulatory domain of 4-1BB optionally comprising the amino acid sequence set forth in SEQ ID NO: 38.

Embodiment 41 provides the nucleic acid of any one of the preceding embodiments, wherein the intracellular domain comprises an intracellular signaling domain of a protein selected from the group consisting of a human CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 42 provides the nucleic acid of any one of the preceding embodiments, wherein the intracellular signaling domain comprises an intracellular signaling domain of CD3 or a variant thereof, optionally comprising the amino acid sequence set forth in SEQ ID NO: 40.

Embodiment 43 provides the nucleic acid of any one of the preceding embodiments, wherein the CAR comprises an anti-CCR4 antigen binding domain comprising an anti-CCR4 scFv, a CD8 alpha hinge, a CD8 alpha transmembrane domain, an intracellular domain comprising a 4-1BB costimulatory domain and a CD3ζ intracellular signaling domain.

Embodiment 44 provides the nucleic acid of embodiment 43, wherein the CAR further comprises a CD8 leader sequence, optionally comprising the amino acid sequence set forth in SEQ ID NO: 32.

Embodiment 45 provides the nucleic acid of any one of the preceding embodiments, wherein the CAR comprises an amino acid sequence at least 80%, 85%, 90%, 95%, 96%, 97%, 98%, 99%, or 100% identical to any one of SEQ ID NOs: 30, 54, 62, 70, 82, 90, and 102.

Embodiment 46 provides the nucleic acid of any one of the preceding embodiments, wherein the CAR is encoded by a nucleotide sequence at least 80%, 85%, 90%, 95%, 96%, 97%, 98%, 99%, or 100% identical to any one of SEQ ID NOs: 29, 53, 61, 69, 81, 89, and 101.

Embodiment 47 provides the nucleic acid of any one of the preceding embodiments, wherein the CAR is linked to a safety switch via a self-cleavable linker, and wherein the safety switch binds a safety switch agent.

Embodiment 48 provides the nucleic acid of embodiment 47, wherein the safety switch is selected from: (a) truncated EGFR (EGFRt), wherein the safety switch agent is an anti-EFGR antibody, optionally cetuximab; (b) CD20, wherein the safety switch agent is an anti-CD20 antibody, optionally rituximab; (c) inducible caspase 9 (iCasp9), wherein the safety switch agent is AP1903; and (d) Herpes simplex virus-1 thymidine kinase (HSVTK), wherein the safety switch agent is ganciclovir (GCV).

Embodiment 49 provides the nucleic acid of embodiment 47 or embodiment 48, wherein the CAR is linked to a dominant negative TGFb receptor type II (dnTGFbRII) via a self-cleavable linker.

Embodiment 50 provides the nucleic acid of embodiment 47 or embodiment 48, wherein the safety switch is linked to a dominant negative TGFb receptor type II (dnTGFbRII) via a self-cleavable linker.

Embodiment 51 provides a vector comprising the nucleic acid of any one of embodiments 26-50.

Embodiment 52 provides the vector of embodiment 51, wherein the vector is a viral vector selected from the group consisting of a retroviral vector, a lentiviral vector, an adenoviral vector, and an adeno-associated viral vector.

Embodiment 53 provides the vector of embodiment 52, wherein the viral vector is a lentiviral vector.

Embodiment 54 provides a modified immune cell or precursor cell thereof, comprising the CAR of any one of embodiments 1-25, the nucleic acid of any one of embodiments 26-50, and/or the vector of any one of embodiments 51-53.

Embodiment 55 provides the modified immune cell or precursor cell thereof of embodiment 54, further comprising one or more of the following;
(a) a CCR4 null knockout allele;
(b) suppressed CCR4 gene expression; and
(c) a fusion protein comprising an anti-CCR4 scFv and a KDEL motif and/or a nucleic acid encoding the fusion protein.

Embodiment 56 provides the modified immune cell or precursor cell thereof of embodiment 55, wherein the CCR4 null knockout allele or suppressed CCR4 gene expression is obtained via a genetic engineering technique comprising a nuclease selected from the group consisting of a clustered regularly interspaced short palindromic repeats (CRISPR) associated nuclease, a transcription activator-like effector nuclease (TALEN), and a zinc-finger nuclease.

Embodiment 57 provides the modified immune cell or precursor cell thereof of embodiment 55 or 56, further comprising an inhibitory RNA molecule which suppresses CCR4 gene expression.

Embodiment 58 provides the modified immune cell or precursor cell thereof of embodiment 57, wherein the inhibitory RNA molecule is selected from the group consisting of: an RNA interference (RNAi) RNA, a short hairpin RNA (shRNA), a small interfering RNA (siRNA), a trans-acting siRNA (tasiRNA), a micro RNA (miRNA), an antisense RNA (asRNA), a long noncoding RNA (lncRNA), a CRISPR RNA (crRNA), a trans-activating crRNA (tracrRNA), a guide RNA (gRNA), a single guide RNA (sgRNA), a double-stranded RNA (dsRNA), a ribozyme, and any combination thereof.

Embodiment 59 provides the modified immune cell or precursor cell thereof of any one of embodiments 54-58, wherein the modified cell is an autologous cell.

Embodiment 60 provides the modified immune cell or precursor cell thereof of any one of embodiments 54-59, wherein the modified immune cell is derived from peripheral blood mononuclear cells (PBMCs).

Embodiment 61 provides the modified immune cell or precursor cell thereof of embodiment 60, wherein the PBMCs comprise tumor cells.

Embodiment 62 provides the modified immune cell or precursor cell thereof of any one of embodiments 54-61, wherein the modified immune cell or precursor cell thereof is a cell isolated from a human subject.

Embodiment 63 provides the modified immune cell or precursor cell thereof of any one of embodiments 54-62, wherein the modified immune cell is a modified T cell.

Embodiment 64 provides a method for generating a modified immune cell or precursor cell thereof, comprising introducing into the immune or precursor cell thereof the nucleic acid of any one of embodiments 26-50 or the vector of any one of embodiment 51-53.

Embodiment 65 provides the method of embodiment 58, wherein the vector is introduced via viral transduction.

Embodiment 66 provides a method of treating cancer in a subject in need thereof, comprising administering to the subject the modified immune or precursor cell of any one of embodiments 54-63, or a modified immune or precursor cell thereof generated by the method of embodiment 64 or embodiment 65.

Embodiment 67 provides the method of embodiment 66, wherein the modified cell depletes CCR4-positive T cells and not CCR4-negative T cells in the subject, thereby treating the cancer.

Embodiment 68 provides the method of embodiment 66 or 67, wherein the subject has previously been administered mogamulizumab.

Embodiment 69 provides the method of any one of embodiments 66-68, wherein the cancer is refractory to mogamulizumab.

Embodiment 70 provides a method of treating cancer in a subject in need thereof, comprising administering to the subject a modified T cell comprising an anti-CCR4 CAR, wherein the CAR comprises an anti-CCR4 antigen binding domain, a transmembrane domain, and an intracellular domain.

Embodiment 71 provides the method of embodiment 70, wherein the anti-CCR4 antigen binding domain comprises at least one heavy chain variable region (HCDR) comprising the amino acid sequence selected from the group consisting of SEQ ID NO: 2, SEQ ID NO: 4, and SEQ ID NO: 6.

Embodiment 72 provides the method of embodiment 70 or embodiment 71, wherein the anti-CCR4 antigen binding domain comprises at least one light chain variable region (LCDR) comprising the amino acid sequence selected from the group consisting of SEQ ID NO: 8, SEQ ID NO: 10, and SEQ ID NO: 12.

Embodiment 73 provides the method of any one of embodiments 70-72, wherein the anti-CCR4 antigen binding domain is selected from the group consisting of a full length antibody or antigen-binding fragment thereof, a Fab, a single-chain variable fragment (scFv), or a single-domain antibody.

Embodiment 74 provides the method of any one of embodiments 70-73, wherein the anti-CCR4 antigen binding domain is an anti-CCR4 single-chain variable fragment (scFv).

Embodiment 75 provides the method of any one of embodiments 70-74, wherein the anti-CCR4 antigen binding domain is an anti-CCR4 single-chain variable fragment (scFv) having a heavy chain and a light chain derived from mogamulizumab.

Embodiment 76 provides the method of any one of embodiments 70-75, wherein the anti-CCR4 antigen binding domain comprises a heavy chain variable region comprising an amino acid sequence at least 80%, 85%, 90%, 95%, 96%, 97%, 98%, 99%, or 100% identical to SEQ ID NO: 112.

Embodiment 77 provides the method of any one of embodiments 70-76, wherein the anti-CCR4 antigen binding domain comprises a light chain variable region comprising an amino acid sequence at least 80%, 85%, 90%, 95%, 96%, 97%, 98%, 99%, or 100% identical to SEQ ID NO: 114.

Embodiment 78 provides the method of any one of embodiments 70-77, wherein the anti-CCR4 antigen binding domain comprises an amino acid sequence at least 80%, 85%, 90%, 95%, 96%, 97%, 98%, 99%, or 100% identical to SEQ ID NO: 16.

Embodiment 79 provides the method of any one of the preceding embodiments, wherein the CAR further comprises a CD8 alpha hinge, optionally wherein the CD8 alpha hinge comprises the amino acid sequence set forth in SEQ ID NO: 34.

Embodiment 80 provides the method of any one of the preceding embodiments, wherein the transmembrane domain comprises a transmembrane domain selected from the group consisting of an artificial hydrophobic sequence, and a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS, and CD154, or a transmembrane domain derived from a killer immunoglobulin-like receptor (KIR).

Embodiment 81 provides the method of any one of the preceding embodiments, wherein the transmembrane domain comprises a CD8 alpha transmembrane domain, optionally wherein the CD8 alpha transmembrane domain comprises the amino acid sequence set forth in SEQ ID NO: 36.

Embodiment 82 provides the method of any one of the preceding embodiments, wherein the intracellular domain comprises a costimulatory domain and an intracellular signaling domain.

Embodiment 83 provides the method of any one of the preceding embodiments, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 84 provides the method of any one of the preceding embodiments, wherein the intracellular domain comprises a costimulatory domain of 4-1BB optionally comprising the amino acid sequence set forth in SEQ ID NO: 38.

Embodiment 85 provides the method of any one of the preceding embodiments, wherein the intracellular domain comprises an intracellular signaling domain of a protein selected from the group consisting of a human CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 86 provides the method of any one of the preceding embodiments, wherein the intracellular signaling domain comprises an intracellular signaling domain of CD3 or a variant thereof, optionally comprising the amino acid sequence set forth in SEQ ID NO: 40.

Embodiment 87 provides the method of any one of the preceding embodiments, wherein the CAR comprises an anti-CCR4 antigen binding domain comprising an anti-CCR4 scFv, a CD8 alpha hinge, a CD8 alpha transmembrane domain, an intracellular domain comprising a 4-1BB costimulatory domain and a CD3ζ intracellular signaling domain.

Embodiment 88 provides the method of embodiment 87, wherein the CAR further comprises a CD8 leader sequence, optionally comprising the amino acid sequence set forth in SEQ ID NO: 32.

Embodiment 89 provides the method of any one of the preceding embodiments, wherein the CAR comprises an amino acid sequence at least 80%, 85%, 90%, 95%, 96%, 97%, 98%, 99%, or 100% identical to any one of SEQ ID NOs: 30, 54, 62, 70, 82, 90, and 102.

Embodiment 90 provides the method of any one of the preceding embodiments, wherein the CAR is encoded by a nucleotide sequence at least 80%, 85%, 90%, 95%, 96%, 97%, 98%, 99%, or 100% identical to any one of SEQ ID NOs: 29, 53, 61, 69, 81, 89, and 101.

Embodiment 91 provides the method of any one of the preceding embodiments, wherein the CAR is linked to a safety switch via a self-cleavable linker, and wherein the safety switch binds a safety switch agent.

Embodiment 92 provides the method of embodiment 91, wherein the safety switch is selected from: (a) truncated EGFR (EGFRt), wherein the safety switch agent is an anti-EFGR antibody, optionally cetuximab; (b) CD20, wherein the safety switch agent is an anti-CD20 antibody, optionally rituximab; (c) inducible caspase 9 (iCasp9), wherein the safety switch agent is AP1903; and (d) Herpes simplex virus-1 thymidine kinase (HSVTK), wherein the safety switch agent is ganciclovir (GCV).

Embodiment 93 provides the method of embodiment 91 or embodiment 92, wherein the CAR is linked to a dominant negative TGFb receptor type II (dnTGFbRII) via a self-cleavable linker.

Embodiment 94 provides the method of embodiment 91 or embodiment 92, wherein the safety switch is linked to a dominant negative TGFb receptor type II (dnTGFbRII) via a self-cleavable linker.

Embodiment 95 provides the method of any one of embodiments 91-94, further comprising administering the safety switch agent to the subject.

Embodiment 96 provides the method of any one of embodiments 70-95, wherein the modified T cell depletes CCR4-positive T cells and not CCR4-negative T cells in the subject, thereby treating the cancer.

Embodiment 97 provides the method of any one of embodiments 70-96, wherein the modified T cell is derived from PBMCs.

Embodiment 98 provides the method of embodiment 97, wherein the PBMCs comprise tumor cells.

Embodiment 99 provides the method of any one of embodiments 70-98, wherein the modified T cell is a human T cell.

Embodiment 100 provides the method of any one of embodiments 70-99, wherein the modified T cell is autologous.

Embodiment 101 provides the method of any one of embodiments 70-100, wherein the subject is human.

Embodiment 102 provides the method of any one of embodiments 70-101, wherein the subject has previously been administered mogamulizumab.

Embodiment 103 provides the method of any one of embodiments 70-102, wherein the cancer is refractory to mogamulizumab.

### OTHER EMBODIMENTS

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An anti-CC chemokine receptor 4 (CCR4) chimeric antigen receptor (CAR) comprising an anti-CCR4 antigen binding domain, a transmembrane domain, and an intracellular domain.

2. The CAR of claim 1, wherein the anti-CCR4 antigen binding domain comprises at least one heavy chain variable region comprising at least one heavy chain complementarity determining region (HCDR) comprising an amino acid sequence selected from the group consisting of SEQ ID NO: 2, SEQ ID NO: 4, and SEQ ID NO: 6.

3. The CAR of claim 1, wherein the anti-CCR4 antigen binding domain comprises at least one light chain variable region comprising at least one light chain complementarity determining region (LCDR) comprising an amino acid sequence selected from the group consisting of SEQ ID NO: 8, SEQ ID NO: 10, and SEQ ID NO: 12.

4-25. (canceled)

26. A nucleic acid comprising a polynucleotide sequence encoding an anti-CCR4 CAR, wherein the CAR comprises an anti-CCR4 antigen binding domain, a transmembrane domain, and an intracellular domain.

27. The nucleic acid of claim 26, wherein the anti-CCR4 antigen binding domain comprises at least one heavy chain variable region comprising at least one heavy chain complementarity determining region (HCDR) comprising an amino acid sequence selected from the group consisting of SEQ ID NO: 2, SEQ ID NO: 4, and SEQ ID NO: 6.

28. The nucleic acid of claim 27, wherein the anti-CCR4 antigen binding domain comprises at least one light chain variable region comprising at least one light chain complementarity determining region (LCDR) comprising an amino acid sequence selected from the group consisting of SEQ ID NO: 8, SEQ ID NO: 10, and SEQ ID NO: 12.

29-50. (canceled)

51. A vector comprising the nucleic acid of claim 26.

52-53. (canceled)

54. A modified immune cell or precursor cell thereof, comprising the CAR of claim 1.

55. The modified immune cell or precursor cell thereof of claim 54, further comprising one or more of the following;
(a) a CCR4 null knockout allele;
(b) suppressed CCR4 gene expression; and
(c) a fusion protein comprising an anti-CCR4 scFv and a KDEL motif and/or a nucleic acid encoding the fusion protein.

56-63. (canceled)

64. A method for generating a modified immune cell or precursor cell thereof, the method comprising introducing into the immune cell or precursor cell thereof the nucleic acid of claim 26.

65. (canceled)

66. A method of treating cancer in a subject in need thereof, the method comprising administering to the subject the modified immune cell or precursor cell of claim 54.

67. The method of claim 66, wherein the modified immune cell or precursor cell depletes CCR4-positive T cells and not CCR4-negative T cells in the subject, thereby treating the cancer.

68. The method of claim 66, wherein the subject has previously been administered mogamulizumab.

69. The method of claim 66, wherein the cancer is refractory to mogamulizumab.

70. A method of treating cancer in a subject in need thereof, the method comprising administering to the subject a modified T cell comprising an anti-CCR4 CAR, wherein the CAR comprises an anti-CCR4 antigen binding domain, a transmembrane domain, and an intracellular domain.

71. The method of claim 70, wherein the anti-CCR4 antigen binding domain comprises at least one heavy chain variable region comprising at least one heavy chain complementarity determining region (HCDR) comprising an amino acid sequence selected from the group consisting of SEQ ID NO: 2, SEQ ID NO: 4, and SEQ ID NO: 6.

72. The method of claim 70, wherein the anti-CCR4 antigen binding domain comprises at least one light chain variable region comprising at least one light chain complementarity determining region (LCDR) comprising the amino acid sequence selected from the group consisting of SEQ ID NO: 8, SEQ ID NO: 10, and SEQ ID NO: 12.

73-95. (canceled)

96. The method of claim 70, wherein the modified T cell depletes CCR4-positive T cells and not CCR4-negative T cells in the subject, thereby treating the cancer.

97-101. (canceled)

102. The method of claim 70, wherein the subject has previously been administered mogamulizumab.

103. The method of claim 70, wherein the cancer is refractory to mogamulizumab.


SEQUENCE LISTING (153 sequences)

SEQ ID NO: 1 - DNA, 15 bp, Artificial Sequence
  CCR4(KW_L2H) HCDR1
aactacggcatgagc

SEQ ID NO: 2 - protein, 5 aa, Artificial Sequence
  CCR4(KW_L2H) HCDR1
NYGMS

SEQ ID NO: 3 - DNA, 51 bp, Artificial Sequence
  CCR4(KW_L2H) HCDR2
acaatcagcagcgccagcacctacagctactaccccgatagcgtgaagggc

SEQ ID NO: 4 - protein, 17 aa, Artificial Sequence
  CCR4(KW_L2H) HCDR2
TISSASTYSYYPDSVKG

SEQ ID NO: 5 - DNA, 30 bp, Artificial Sequence
  CCR4(KW_L2H) HCDR3
cacagcgacggcaacttcgcctttggctat

SEQ ID NO: 6 - protein, 10 aa, Artificial Sequence
  CCR4(KW_L2H) HCDR3
HSDGNFAFGY

SEQ ID NO: 7 - DNA, 48 bp, Artificial Sequence
  CCR4(KW_L2H) LCDR1
agatccagccggaacatcgtgcacatcaacggcgacacctacctggaa

SEQ ID NO: 8 - protein, 16 aa, Artificial Sequence
  CCR4(KW_L2H) LCDR1
RSSRNIVHINGDTYLE

SEQ ID NO: 9 - DNA, 21 bp, Artificial Sequence
  CCR4(KW_L2H) LCDR2
aaggtgtccaaccggttcagc

SEQ ID NO: 10 - protein, 7 aa, Artificial Sequence
  CCR4(KW_L2H) LCDR2
KVSNRFS

SEQ ID NO: 11 - DNA, 27 bp, Artificial Sequence
  CCR4(KW_L2H) LCDR3
ttccaaggctctctgctgccctggaca

SEQ ID NO: 12 - protein, 9 aa, Artificial Sequence
  CCR4(KW_L2H) LCDR3
FQGSLLPWT

SEQ ID NO: 13 - DNA, 753 bp, Artificial Sequence
  CCR4(KW_H2L) scFv
gaagtgcagctggttgagtctggcggagatctggtgcagcctggcagaagcctgagactgtcttgtgccgccagcggcttcatcttcagcaactacggcatgagctgggtccgacaggcccctggaaaaggccttgaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactactgcggcagacacagcgacggcaatttcgcctttggctattggggccagggcaccctggttaccgtttctagcggaggaggtggatcaggtggaggtggaagcgggggaggaggttccggcggcggaggatcagatgttctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtctactactgcttccaaggctctctgctgccctggacatttggacagggcaccaaggtggaaatcaag

SEQ ID NO: 14 - protein, 251 aa, Artificial Sequence
  CCR4(KW_H2L) scFv
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSGGGGSGGGGSGGGGSGGGGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 15 - DNA, 753 bp, Artificial Sequence
  CCR4(KW_L2H) scFv
gacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagt

SEQ ID NO: 16 - protein, 251 aa, Artificial Sequence
  CCR4(KW_L2H) scFv
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSS

SEQ ID NO: 17 - DNA, 750 bp, Artificial Sequence
  CCR4(1-49_H2L) scFv
caggtgcagctggtgcagagcggagccgaggtgaagaagcctggagcttccgtcaaggtgtcctgcaaggccagcggctacaccttcgccagcagctggatgcactggatgcggcaggcacctggacagggcctcgaatggatcggctggatcaaccccggcaacgtgaacaccaagtacaacgagaagttcaagggcagggccaccctgaccgtggacaccagcaccaacaccgcctacatggaactgagcagcctgcggagcgaggacaccgccgtgtactactgcgccagaagcacgtggtatcggccgaatgactactggggccagggcaccctggtgaccgtgagcagcggaggaggtggatcaggtggaggtggaagcgggggaggaggttccggcggcggaggatcagacatcgtgatgacccagagccccgacagcctggccgtgagcctgggcgagcgggccaccatcaactgcaagagcagccagagcatcctgtacagcagcaaccagaagaactacctggcctggtatcagcagaagcccggccagagccccaagctgctgatctactgggccagcacccgggagagcggcgtgcccgaccggtttagcggcagcggctccggcaccgacttcaccctgaccatcagcagcctgcaggccgaggacgtggccgtgtactactgccaccagtacaaaagcagctacaccttcggccagggcacaaagctggaaatcaag

SEQ ID NO: 18 - protein, 250 aa, Artificial Sequence
  CCR4(1-49_H2L) scFv
QVQLVQSGAEVKKPGASVKVSCKASGYTFASSWMHWMRQAPGQGLEWIGWINPGNVNTKYNEKFKGRATLTVDTSTNTAYMELSSLRSEDTAVYYCARSTWYRPNDYWGQGTLVTVSSGGGGSGGGGSGGGGSGGGGSDIVMTQSPDSLAVSLGERATINCKSSQSILYSSNQKNYLAWYQQKPGQSPKLLIYWASTRESGVPDRFSGSGSGTDFTLTISSLQAEDVAVYYCHQYKSSYTFGQGTKLEIK

SEQ ID NO: 19 - DNA, 750 bp, Artificial Sequence
  CCR4(1-49_L2H) scFv
gacatcgtgatgacccagagccccgacagcctggccgtgagcctgggcgagcgggccaccatcaactgcaagagcagccagagcatcctgtacagcagcaaccagaagaactacctggcctggtatcagcagaagcccggccagagccccaagctgctgatctactgggccagcacccgggagagcggcgtgcccgaccggtttagcggcagcggctccggcaccgacttcaccctgaccatcagcagcctgcaggccgaggacgtggccgtgtactactgccaccagtacaaaagcagctacaccttcggccagggcacaaagctggaaatcaagggaggaggtggatcaggtggaggtggaagcgggggaggaggttccggcggcggaggatcacaggtgcagctggtgcagagcggagccgaggtgaagaagcctggagcttccgtcaaggtgtcctgcaaggccagcggctacaccttcgccagcagctggatgcactggatgcggcaggcacctggacagggcctcgaatggatcggctggatcaaccccggcaacgtgaacaccaagtacaacgagaagttcaagggcagggccaccctgaccgtggacaccagcaccaacaccgcctacatggaactgagcagcctgcggagcgaggacaccgccgtgtactactgcgccagaagcacgtggtatcggccgaatgactactggggccagggcaccctggtgaccgtgagcagc

SEQ ID NO: 20 - protein, 250 aa, Artificial Sequence
  CCR4(1-49_L2H) scFv
DIVMTQSPDSLAVSLGERATINCKSSQSILYSSNQKNYLAWYQQKPGQSPKLLIYWASTRESGVPDRFSGSGSGTDFTLTISSLQAEDVAVYYCHQYKSSYTFGQGTKLEIKGGGGSGGGGSGGGGSGGGGSQVQLVQSGAEVKKPGASVKVSCKASGYTFASSWMHWMRQAPGQGLEWIGWINPGNVNTKYNEKFKGRATLTVDTSTNTAYMELSSLRSEDTAVYYCARSTWYRPNDYWGQGTLVTVSS

SEQ ID NO: 21 - DNA, 750 bp, Artificial Sequence
  CCR4(2-3_H2L) scFv
caggtgcagctggtgcagagcggagccgaggtgaagaagcctggagcttccgtcaaggtgtcctgcaaggccagcggctacaccttcgccagcgcgtggatgcactggatgcggcaggcacctggacagggcctcgaatggatcggctggatcaaccccggcaacgtgaacaccaagtacaacgagaagttcaagggcagggccaccctgaccgtggacaccagcaccaacaccgcctacatggaactgagcagcctgcggagcgaggacaccgccgtgtactactgcgccagaagcacgtattaccggccgctggactactggggccagggcaccctggtgaccgtgagcagcggaggaggtggatcaggtggaggtggaagcgggggaggaggttccggcggcggaggatcagacatcgtgatgacccagagccccgacagcctggccgtgagcctgggcgagcgggccaccatcaactgcaagagcagccagagcatcctgtacagcagcaaccagaagaactacctggcctggtatcagcagaagcccggccagagccccaagctgctgatctactgggccagcacccgggagagcggcgtgcccgaccggtttagcggcagcggctccggcaccgacttcaccctgaccatcagcagcctgcaggccgaggacgtggccgtgtactactgccaccagtacatgagcagctacaccttcggccagggcacaaagctggaaatcaag

SEQ ID NO: 22 - protein, 250 aa, Artificial Sequence
  CCR4(2-3_H2L) scFv
QVQLVQSGAEVKKPGASVKVSCKASGYTFASAWMHWMRQAPGQGLEWIGWINPGNVNTKYNEKFKGRATLTVDTSTNTAYMELSSLRSEDTAVYYCARSTYYRPLDYWGQGTLVTVSSGGGGSGGGGSGGGGSGGGGSDIVMTQSPDSLAVSLGERATINCKSSQSILYSSNQKNYLAWYQQKPGQSPKLLIYWASTRESGVPDRFSGSGSGTDFTLTISSLQAEDVAVYYCHQYMSSYTFGQGTKLEIK

SEQ ID NO: 23 - DNA, 750 bp, Artificial Sequence
  CCR4(2-3_L2H) scFv
gacatcgtgatgacccagagccccgacagcctggccgtgagcctgggcgagcgggccaccatcaactgcaagagcagccagagcatcctgtacagcagcaaccagaagaactacctggcctggtatcagcagaagcccggccagagccccaagctgctgatctactgggccagcacccgggagagcggcgtgcccgaccggtttagcggcagcggctccggcaccgacttcaccctgaccatcagcagcctgcaggccgaggacgtggccgtgtactactgccaccagtacatgagcagctacaccttcggccagggcacaaagctggaaatcaagggaggaggtggatcaggtggaggtggaagcgggggaggaggttccggcggcggaggatcacaggtgcagctggtgcagagcggagccgaggtgaagaagcctggagcttccgtcaaggtgtcctgcaaggccagcggctacaccttcgccagcgcgtggatgcactggatgcggcaggcacctggacagggcctcgaatggatcggctggatcaaccccggcaacgtgaacaccaagtacaacgagaagttcaagggcagggccaccctgaccgtggacaccagcaccaacaccgcctacatggaactgagcagcctgcggagcgaggacaccgccgtgtactactgcgccagaagcacgtattaccggccgctggactactggggccagggcaccctggtgaccgtgagcagc

SEQ ID NO: 24 - protein, 250 aa, Artificial Sequence
  CCR4(2-3_L2H) scFv
DIVMTQSPDSLAVSLGERATINCKSSQSILYSSNQKNYLAWYQQKPGQSPKLLIYWASTRESGVPDRFSGSGSGTDFTLTISSLQAEDVAVYYCHQYMSSYTFGQGTKLEIKGGGGSGGGGSGGGGSGGGGSQVQLVQSGAEVKKPGASVKVSCKASGYTFASAWMHWMRQAPGQGLEWIGWINPGNVNTKYNEKFKGRATLTVDTSTNTAYMELSSLRSEDTAVYYCARSTYYRPLDYWGQGTLVTVSS

SEQ ID NO: 25 - DNA, 750 bp, Artificial Sequence
  CCR4(h1567_H2L) scFv
caggtgcagctggtgcagagcggagccgaggtgaagaagcctggagcttccgtcaaggtgtcctgcaaggccagcggctacaccttcgccagctactacatgcactggatgcggcaggcacctggacagggcctcgaatggatcggctggatcaaccccggcaacgtgaacaccaagtacaacgagaagttcaagggcagggccaccctgaccgtggacaccagcaccaacaccgcctacatggaactgagcagcctgcggagcgaggacaccgccgtgtactactgcgccagaagcacctactaccggcccctggactactggggccagggcaccctggtgaccgtgagcagcggaggaggtggatcaggtggaggtggaagcgggggaggaggttccggcggcggaggatcagacatcgtgatgacccagagccccgacagcctggccgtgagcctgggcgagcgggccaccatcaactgcaagagcagccagagcatcctgtacagcagcaaccagaagaactacctggcctggtatcagcagaagcccggccagagccccaagctgctgatctactgggccagcacccgggagagcggcgtgcccgaccggtttagcggcagcggctccggcaccgacttcaccctgaccatcagcagcctgcaggccgaggacgtggccgtgtactactgccaccagtacctgagcagctacaccttcggccagggcacaaagctggaaatcaag

SEQ ID NO: 26 - protein, 250 aa, Artificial Sequence
  CCR4(h1567_H2L) scFv
QVQLVQSGAEVKKPGASVKVSCKASGYTFASYYMHWMRQAPGQGLEWIGWINPGNVNTKYNEKFKGRATLTVDTSTNTAYMELSSLRSEDTAVYYCARSTYYRPLDYWGQGTLVTVSSGGGGSGGGGSGGGGSGGGGSDIVMTQSPDSLAVSLGERATINCKSSQSILYSSNQKNYLAWYQQKPGQSPKLLIYWASTRESGVPDRFSGSGSGTDFTLTISSLQAEDVAVYYCHQYLSSYTFGQGTKLEIK

SEQ ID NO: 27 - DNA, 750 bp, Artificial Sequence
  CCR4(h1567_L2H) scFv
gacatcgtgatgacccagagccccgacagcctggccgtgagcctgggcgagcgggccaccatcaactgcaagagcagccagagcatcctgtacagcagcaaccagaagaactacctggcctggtatcagcagaagcccggccagagccccaagctgctgatctactgggccagcacccgggagagcggcgtgcccgaccggtttagcggcagcggctccggcaccgacttcaccctgaccatcagcagcctgcaggccgaggacgtggccgtgtactactgccaccagtacctgagcagctacaccttcggccagggcacaaagctggaaatcaagggaggaggtggatcaggtggaggtggaagcgggggaggaggttccggcggcggaggatcacaggtgcagctggtgcagagcggagccgaggtgaagaagcctggagcttccgtcaaggtgtcctgcaaggccagcggctacaccttcgccagctactacatgcactggatgcggcaggcacctggacagggcctcgaatggatcggctggatcaaccccggcaacgtgaacaccaagtacaacgagaagttcaagggcagggccaccctgaccgtggacaccagcaccaacaccgcctacatggaactgagcagcctgcggagcgaggacaccgccgtgtactactgcgccagaagcacctactaccggcccctggactactggggccagggcaccctggtgaccgtgagcagc

SEQ ID NO: 28 - protein, 250 aa, Artificial Sequence
  CCR4(h1567_L2H) scFv
DIVMTQSPDSLAVSLGERATINCKSSQSILYSSNQKNYLAWYQQKPGQSPKLLIYWASTRESGVPDRFSGSGSGTDFTLTISSLQAEDVAVYYCHQYLSSYTFGQGTKLEIKGGGGSGGGGSGGGGSGGGGSQVQLVQSGAEVKKPGASVKVSCKASGYTFASYYMHWMRQAPGQGLEWIGWINPGNVNTKYNEKFKGRATLTVDTSTNTAYMELSSLRSEDTAVYYCARSTYYRPLDYWGQGTLVTVSS

SEQ ID NO: 29 - DNA, 1500 bp, Artificial Sequence
  CCR4(KW_L2H) CAR
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 30 - protein, 499 aa, Artificial Sequence
  CCR4(KW_L2H) CAR
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 63 bp, Artificial Sequence
  CD8 leader
atggccctgcctgtgacagccctgctgctgcctctggctctgctgctgcatgccgctagaccc

SEQ ID NO: 32 - protein, 21 aa, Artificial Sequence
  CD8 leader
MALPVTALLLPLALLLHAARP

SEQ ID NO: 33 - DNA, 135 bp, Artificial Sequence
  CD8 hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 34 - protein, 45 aa, Artificial Sequence
  CD8 hinge
TTTPAPRPPTPAPTIASQPLSLRPEACRPAAGGAVHTRGLDFACD

SEQ ID NO: 35 - DNA, 72 bp, Artificial Sequence
  CD8 transmembrane
atctacatctgggcgcccttggccgggacttgtggggtccttctcctgtcactggttatcaccctttactgc

SEQ ID NO: 36 - protein, 24 aa, Artificial Sequence
  CD8 transmembrane
IYIWAPLAGTCGVLLLSLVITLYC

SEQ ID NO: 37 - DNA, 126 bp, Artificial Sequence
  4-1BB intracellular domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 38 - protein, 42 aa, Artificial Sequence
  4-1BB intracellular domain
KRGRKKLLYIFKQPFMRPVQTTQEEDGCSCRFPEEEEGGCEL

SEQ ID NO: 39 - DNA, 339 bp, Artificial Sequence
  CD3 zeta
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 40 - protein, 112 aa, Artificial Sequence
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 2643 bp, Artificial Sequence
  CCR4(KW_L2H)-tEGFR
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccatatgggaagcggagctactaacttcagcctgctgaagcaggctggagacgtggaggagaaccctggacctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 42 - protein, 880 aa, Artificial Sequence
  CCR4(KW_L2H)-tEGFR
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHMGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 43 - DNA, 1503 bp, Artificial Sequence
  CCR4(KW_L2H)
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccatatg

SEQ ID NO: 44 - protein, 501 aa, Artificial Sequence
  CCR4(KW_L2H)
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHM

SEQ ID NO: 45 - DNA, 66 bp, Artificial Sequence
  P2A
ggaagcggagctactaacttcagcctgctgaagcaggctggagacgtggaggagaaccctggacct

SEQ ID NO: 46 - protein, 22 aa, Artificial Sequence
  P2A
GSGATNFSLLKQAGDVEENPGP

SEQ ID NO: 47 - DNA, 66 bp, Artificial Sequence
  GMSCFR leader
atgcttctcctggtgacaagccttctgctctgtgagttaccacacccagcattcctcctgatccca

SEQ ID NO: 48 - protein, 22 aa, Artificial Sequence
  GMSCFR leader
MLLLVTSLLLCELPHPAFLLIP

SEQ ID NO: 49 - DNA, 1008 bp, Artificial Sequence
  tEGFR
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 50 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 51 - DNA, 2463 bp, Artificial Sequence
  CCR4(KW_L2H)-CD20
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccatatgggaagcggagctactaacttcagcctgctgaagcaggctggagacgtggaggagaaccctggacctatgacaacacccagaaattcagtaaatgggactttcccggcagagccaatgaaaggccctattgctatgcaatctggtccaaaaccactcttcaggaggatgtcttcactggtgggccccacgcaatccttcttcatgagggaatctaagactttgggggctgtccagattatgaatgggctcttccacattgccctggggggtcttctgatgatcccagcagggatctatgcacccatctgtgtgactgtgtggtatcctctctggggaggcattatgtatattatttccggatcactcctggcagcaacggagaaaaactccaggaagtgtttggtcaaaggaaaaatgataatgaactcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 52 - protein, 820 aa, Artificial Sequence
  CCR4(KW_L2H)-CD20
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHMGSGATNFSLLKQAGDVEENPGPMTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 53 - DNA, 1503 bp, Artificial Sequence; identical to SEQ ID NO: 43
  CCR4(KW_L2H)

SEQ ID NO: 54 - protein, 501 aa, Artificial Sequence; identical to SEQ ID NO: 44
  CCR4(KW_L2H)

SEQ ID NO: 55 - DNA, 66 bp, Artificial Sequence; identical to SEQ ID NO: 45
  P2A

SEQ ID NO: 56 - protein, 22 aa, Artificial Sequence
  P2A
GSGATNFSLLKQAGDVEENPGP

SEQ ID NO: 57 - DNA, 894 bp, Artificial Sequence
  CD20
atgacaacacccagaaattcagtaaatgggactttcccggcagagccaatgaaaggccctattgctatgcaatctggtccaaaaccactcttcaggaggatgtcttcactggtgggccccacgcaatccttcttcatgagggaatctaagactttgggggctgtccagattatgaatgggctcttccacattgccctggggggtcttctgatgatcccagcagggatctatgcacccatctgtgtgactgtgtggtatcctctctggggaggcattatgtatattatttccggatcactcctggcagcaacggagaaaaactccaggaagtgtttggtcaaaggaaaaatgataatgaactcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 58 - protein, 297 aa, Artificial Sequence
  CD20
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 59 - DNA, 2754 bp, Artificial Sequence
  CCR4(KW_L2H)-FKBP-iCasp9
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccatatgggaagcggagctactaacttcagcctgctgaagcaggctggagacgtggaggagaaccctggacctggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggctccggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 60 - protein, 917 aa, Artificial Sequence
  CCR4(KW_L2H)-FKBP-iCasp9
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHMGSGATNFSLLKQAGDVEENPGPGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 61 - DNA, 1503 bp, Artificial Sequence; identical to SEQ ID NO: 43
  CCR4(KW_L2H)

SEQ ID NO: 62 - protein, 501 aa, Artificial Sequence; identical to SEQ ID NO: 44
  CCR4(KW_L2H)

SEQ ID NO: 63 - DNA, 66 bp, Artificial Sequence; identical to SEQ ID NO: 45
  P2A

SEQ ID NO: 64 - protein, 22 aa, Artificial Sequence
  P2A
GSGATNFSLLKQAGDVEENPGP

SEQ ID NO: 65 - DNA, 1185 bp, Artificial Sequence
  FKBP-iCasp9
ggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggctccggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 66 - protein, 394 aa, Artificial Sequence
  FKBP-iCasp9
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 67 - DNA, 2694 bp, Artificial Sequence
  CCR4(KW_L2H)-HSVTK
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggcgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactaa

SEQ ID NO: 68 - protein, 897 aa, Artificial Sequence
  CCR4(KW_L2H)-HSVTK
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 69 - DNA, 1503 bp, Artificial Sequence; identical to SEQ ID NO: 43
  CCR4(KW_L2H)

SEQ ID NO: 70 - protein, 501 aa, Artificial Sequence; identical to SEQ ID NO: 44
  CCR4(KW_L2H)

SEQ ID NO: 71 - DNA, 66 bp, Artificial Sequence; identical to SEQ ID NO: 45
  P2A

SEQ ID NO: 72 - protein, 22 aa, Artificial Sequence
  P2A
GSGATNFSLLKQAGDVEENPGP

SEQ ID NO: 73 - DNA, 1131 bp, Artificial Sequence
  HSVTK
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggcgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactaa

SEQ ID NO: 74 - protein, 376 aa, Artificial Sequence
  HSVTK
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 75 - DNA, 3423 bp, Artificial Sequence
  dnTGFRII-CCR4(KW_L2H)-HSVTK
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgggcagcggcgagggcagaggcagcctgctgacctgcggcgacgtggaggagaaccccggacctatggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggcgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactaa

SEQ ID NO: 76 - protein, 1140 aa, Artificial Sequence
  dnTGFRII-CCR4(KW_L2H)-HSVTK
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLGSGEGRGSLLTCGDVEENPGPMALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 77 - DNA, 666 bp, Artificial Sequence
  dnTGNbRII
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctg

SEQ ID NO: 78 - protein, 222 aa, Artificial Sequence
  dnTGNbRII
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKL

SEQ ID NO: 79 - DNA, 63 bp, Artificial Sequence
  T2A
ggcagcggcgagggcagaggcagcctgctgacctgcggcgacgtggaggagaaccccggacct

SEQ ID NO: 80 - protein, 21 aa, Artificial Sequence
  T2A
GSGEGRGSLLTCGDVEENPGP

SEQ ID NO: 81 - DNA, 1497 bp, Artificial Sequence
  CCR4(KW_L2H)
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 82 - protein, 499 aa, Artificial Sequence; identical to SEQ ID NO: 30
  CCR4(KW_L2H)

SEQ ID NO: 83 - DNA, 66 bp, Artificial Sequence; identical to SEQ ID NO: 45
  P2A

SEQ ID NO: 84 - protein, 22 aa, Artificial Sequence
  P2A
GSGATNFSLLKQAGDVEENPGP

SEQ ID NO: 85 - DNA, 1131 bp, Artificial Sequence; identical to SEQ ID NO: 73
  HSVTK

SEQ ID NO: 86 - protein, 376 aa, Artificial Sequence; identical to SEQ ID NO: 74
  HSVTK

SEQ ID NO: 87 - DNA, 3423 bp, Artificial Sequence
  CCR4(KW_L2H)-dnTGFbRII-HSVTK
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgggcagcggcgagggcagaggcagcctgctgacctgcggcgacgtggaggagaaccccggacctatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggcgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactaa

SEQ ID NO: 88 - protein, 1140 aa, Artificial Sequence
  CCR4(KW_L2H)-dnTGFbRII-HSVTK
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLGSGEGRGSLLTCGDVEENPGPMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 89 - DNA, 1497 bp, Artificial Sequence; identical to SEQ ID NO: 81
  CCR4(KW_L2H)

SEQ ID NO: 90 - protein, 499 aa, Artificial Sequence; identical to SEQ ID NO: 30
  CCR4(KW_L2H)

SEQ ID NO: 91 - DNA, 66 bp, Artificial Sequence; identical to SEQ ID NO: 45
  P2A

SEQ ID NO: 92 - protein, 22 aa, Artificial Sequence
  P2A
GSGATNFSLLKQAGDVEENPGP

SEQ ID NO: 93 - DNA, 666 bp, Artificial Sequence; identical to SEQ ID NO: 77
  dnTGNbRII

SEQ ID NO: 94 - protein, 222 aa, Artificial Sequence; identical to SEQ ID NO: 78
  dnTGNbRII

SEQ ID NO: 95 - DNA, 63 bp, Artificial Sequence; identical to SEQ ID NO: 79
  T2A

SEQ ID NO: 96 - protein, 21 aa, Artificial Sequence
  T2A
GSGEGRGSLLTCGDVEENPGP

SEQ ID NO: 97 - DNA, 1131 bp, Artificial Sequence; identical to SEQ ID NO: 73
  HSVTK

SEQ ID NO: 98 - protein, 376 aa, Artificial Sequence; identical to SEQ ID NO: 74
  HSVTK

SEQ ID NO: 99 - DNA, 3423 bp, Artificial Sequence
  CCR4(KW_L2H)-HSVTK-dnTGFbRII
atggccctgcctgtgacagccctgctgctgcctctggctctgctgctgcatgccgctagacccggatccgacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaagggaggaggtggatcaggtggaggtggaagcgggggaggaggttccggcggcggaggatcagaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggcgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaacggcagcggcgagggcagaggcagcctgctgacctgcggcgacgtggaggagaaccccggacctatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgtaa

SEQ ID NO: 100 - protein, 1140 aa, Artificial Sequence
  CCR4(KW_L2H)-HSVTK-dnTGFbRII
MALPVTALLLPLALLLHAARPGSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKGGGGSGGGGSGGGGSGGGGSEVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKL

SEQ ID NO: 101 - DNA, 1497 bp, Artificial Sequence; identical to SEQ ID NO: 81
  CCR4(KW_L2H)

SEQ ID NO: 102 - protein, 499 aa, Artificial Sequence; identical to SEQ ID NO: 30
  CCR4(KW_L2H)

SEQ ID NO: 103 - DNA, 66 bp, Artificial Sequence; identical to SEQ ID NO: 45
  P2A

SEQ ID NO: 104 - protein, 22 aa, Artificial Sequence
  P2A
GSGATNFSLLKQAGDVEENPGP

SEQ ID NO: 105 - DNA, 1128 bp, Artificial Sequence
  HSVTK
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggcgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaac

SEQ ID NO: 106 - protein, 376 aa, Artificial Sequence; identical to SEQ ID NO: 74
  HSVTK

SEQ ID NO: 107 - DNA, 63 bp, Artificial Sequence; identical to SEQ ID NO: 79
  T2A

SEQ ID NO: 108 - protein, 21 aa, Artificial Sequence
  T2A
GSGEGRGSLLTCGDVEENPGP

SEQ ID NO: 109 - DNA, 669 bp, Artificial Sequence
  dnTGNbRII
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgtaa

SEQ ID NO: 110 - protein, 222 aa, Artificial Sequence; identical to SEQ ID NO: 78
  dnTGNbRII

SEQ ID NO: 111 - DNA, 357 bp, Artificial Sequence
  CCR4(KW_L2H) VH chain
gaagttcagctggttgagtctggcggcgacctggttcagcctggcagatctctgagactgagctgtgccgccagcggcttcatcttcagcaactacggcatgagctgggtccgacaggcccctggaaaaggactggaatgggtcgccacaatcagcagcgccagcacctacagctactaccccgatagcgtgaagggcagattcaccatcagccgggacaacgccaagaacagcctgtacctgcagatgaactccctgcgcgtggaagatacagccctgtactattgcggcagacacagcgacggcaacttcgcctttggctattggggccagggaaccctggtcaccgtttctagt

SEQ ID NO: 112 - protein, 119 aa, Artificial Sequence
  CCR4(KW_L2H) VH chain
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSS

SEQ ID NO: 113 - DNA, 336 bp, Artificial Sequence
  CCR4(KW_L2H) VL chain
gacgtgctgatgacacagagccctctgagcctgcctgtgacacctggcgaacctgccagcatcagctgcagatccagccggaacatcgtgcacatcaacggcgacacctacctggaatggtatctgcagaagcccggccagtctcctcagctgctgatctacaaggtgtccaaccggttcagcggcgtgcccgatagattttctggcagcggctctggcaccgacttcaccctgaagatctccagagtggaagccgaggacgtgggcgtgtactactgcttccaaggctctctgctgccctggacatttggccagggcaccaaggtggaaatcaag

SEQ ID NO: 114 - protein, 112 aa, Artificial Sequence
  CCR4(KW_L2H) VL chain
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 115 - protein, 5 aa, Artificial Sequence
  GS Linker
  REPEAT (1)..(5): Sequence repeats N times, where N = an integer of at least 1.
GSGGS

SEQ ID NO: 116 - protein, 4 aa, Artificial Sequence
  GS Linker
  REPEAT (1)..(4): Sequence repeats N times, where N = an integer of at least 1.
GGGS

SEQ ID NO: 117 - protein, 5 aa, Artificial Sequence
  GS Linker
  REPEAT (1)..(5): Sequence repeats N times, where N = an integer of at least 1.
GGGGS

SEQ ID NO: 118 - protein, 4 aa, Artificial Sequence
  GS Linker
GGSG

SEQ ID NO: 119 - protein, 5 aa, Artificial Sequence
  GS Linker
GGSGG

SEQ ID NO: 120 - protein, 5 aa, Artificial Sequence
  GS Linker
GSGSG

SEQ ID NO: 121 - protein, 5 aa, Artificial Sequence
  GS Linker
GSGGG

SEQ ID NO: 122 - protein, 5 aa, Artificial Sequence
  GS Linker
GGGSG

SEQ ID NO: 123 - protein, 5 aa, Artificial Sequence
  GS Linker
GSSSG

SEQ ID NO: 124 - protein, 5 aa, Artificial Sequence
  GS Linker
GGGGS

SEQ ID NO: 125 - protein, 20 aa, Artificial Sequence
  GS Linker
GGGGSGGGGSGGGGSGGGGS

SEQ ID NO: 126 - DNA, 60 bp, Artificial Sequence
  GS Linker
ggaggaggtggatcaggtggaggtggaagcgggggaggaggttccggcggcggaggatca

SEQ ID NO: 127 - protein, 4 aa, Artificial Sequence
  Furin Cleavage Site
  MISC_FEATURE (2)..(2): Xaa is any naturally occurring amino acid
RXKR

SEQ ID NO: 128 - protein, 4 aa, Artificial Sequence
  Furin Cleavage Site
  MISC_FEATURE (2)..(2): Xaa is any naturally occurring amino acid
RXRR

SEQ ID NO: 129 - protein, 5 aa, Artificial Sequence
  Furin Cleavage Site
  MISC_FEATURE (1)..(1): Xaa is Lys or Arg
  MISC_FEATURE (3)..(3): Xaa is any naturally occuring amino acid
  MISC_FEATURE (4)..(4): Xaa is Lys or Arg
XRXXR

SEQ ID NO: 130 - protein, 4 aa, Artificial Sequence
  Furin Cleavage Site
  MISC_FEATURE (2)..(2): Xaa is any naturally occurring amino acid
  MISC_FEATURE (3)..(3): Xaa is any naturally occurring amino acid
RXXR

SEQ ID NO: 131 - protein, 4 aa, Artificial Sequence
  Furin Cleavage Site
RQKR

SEQ ID NO: 132 - protein, 5 aa, Artificial Sequence
  Hinge
DKTHT

SEQ ID NO: 133 - protein, 4 aa, Artificial Sequence
  Hinge
CPPC

SEQ ID NO: 134 - protein, 15 aa, Artificial Sequence
  Hinge
CPEPKSCDTPPPCPR

SEQ ID NO: 135 - protein, 12 aa, Artificial Sequence
  Hinge
ELKTPLGDTTHT

SEQ ID NO: 136 - protein, 10 aa, Artificial Sequence
  Hinge
KSCDKTHTCP

SEQ ID NO: 137 - protein, 7 aa, Artificial Sequence
  Hinge
KCCVDCP

SEQ ID NO: 138 - protein, 7 aa, Artificial Sequence
  Hinge
KYGPPCP

SEQ ID NO: 139 - protein, 15 aa, Artificial Sequence
  Hinge
EPKSCDKTHTCPPCP

SEQ ID NO: 140 - protein, 12 aa, Artificial Sequence
  Hinge
ERKCCVECPPCP

SEQ ID NO: 141 - protein, 17 aa, Artificial Sequence
  Hinge
ELKTPLGDTTHTCPRCP

SEQ ID NO: 142 - protein, 12 aa, Artificial Sequence
  Hinge
SPNMVPHAHHAQ

SEQ ID NO: 143 - protein, 15 aa, Artificial Sequence
  Hinge
EPKSCDKTYTCPPCP

SEQ ID NO: 144 - RNA, 67 nt, Artificial Sequence
  sh-CCR4#1
gaaugaaguuguagguaauaucugugaagccacagaugggauauuaccuacaacuucauucuuuuuu

SEQ ID NO: 145 - RNA, 67 nt, Artificial Sequence
  sh-CCR4#2
gcaugagucagucugaugagacugugaagccacagaugggucucaucagacugacucaugcuuuuuu

SEQ ID NO: 146 - RNA, 67 nt, Artificial Sequence
  sh-CCR4#3
gcugauggaguaaaucgcuaccugugaagccacagaugggguagcgauuuacuccaucagcuuuuuu

SEQ ID NO: 147 - RNA, 67 nt, Artificial Sequence
  sh-CCR4#4
guuguagguaauauugcaaggcugugaagccacagaugggccuugcaauauuaccuacaacuuuuuu

SEQ ID NO: 148 - RNA, 67 nt, Artificial Sequence
  sh-Cre
gauuuacggcgcuaaggaugacugugaagccacagaugggucauccuuagcgccguaaaucuuuuuu

SEQ ID NO: 149 - DNA, 67 bp, Artificial Sequence
  DNA encoding sh-CCR4#1
gaatgaagttgtaggtaatatctgtgaagccacagatgggatattacctacaacttcattctttttt

SEQ ID NO: 150 - DNA, 67 bp, Artificial Sequence
  DNA encoding sh-CCR4#2
gcatgagtcagtctgatgagactgtgaagccacagatgggtctcatcagactgactcatgctttttt

SEQ ID NO: 151 - DNA, 67 bp, Artificial Sequence
  DNA encoding sh-CCR4#3
gctgatggagtaaatcgctacctgtgaagccacagatggggtagcgatttactccatcagctttttt

SEQ ID NO: 152 - DNA, 67 bp, Artificial Sequence
  DNA encoding sh-CCR4#4
gttgtaggtaatattgcaaggctgtgaagccacagatgggccttgcaatattacctacaactttttt

SEQ ID NO: 153 - DNA, 67 bp, Artificial Sequence
  DNA encoding sh-Cre
gatttacggcgctaaggatgactgtgaagccacagatgggtcatccttagcgccgtaaatctttttt